(19) United States
(12) Patent Application Publication
(10) Pub. No.: US 2025/0319024 A1
(43) Pub. Date: Oct. 16, 2025

(54) TARGETED NANOPARTICLES AND THEIR USES RELATED TO INFECTIOUS DISEASE

(71) Applicant: UNIVERSITY OF GEORGIA RESEARCH FOUNDATION, INC., Athens, GA (US)

(72) Inventors: Richard Meagher, Athens, GA (US); Suresh Ambati, Athens, GA (US); Zachary Lewis, Athens, GA (US); Xiaorong Lin, Athens, GA (US)

(73) Assignee: UNIVERSITY OF GEORGIA RESEARCH FOUNDATION, INC., Athems, GA (US)

(21) Appl. No.: 18/686,113

(22) PCT Filed: Aug. 26, 2022
(86) PCT No.: PCT/US2022/075530

§ 371 Date: Feb. 23, 2024

Related U.S. Application Data
(60) Provisional application No. 63/237,687, filed on Aug. 27, 2021.

(51) Int. Cl. A61K 9/1271; A61K 38/17; A61K 45/06; A61P 31/04; A61P 31/06; A61P 31/10; A61P 31/14; A61P 33/02

(52) U.S. Cl. CPC A61K 9/1271; A61K 38/178; A61K 45/06; A61P 31/04; A61P 31/06; A61P 31/10; A61P 31/14; A61P 33/02

(57) ABSTRACT

Infectious diseases continue to burden populations around the world. Both naturally occurring and engineered biological threats hold increasing potential to cause disease, disability, and death. The liposomes comprise a targeting molecule that binds a target antigen expressed by a pathogen, wherein the targeting molecule is a C-Type Lectin polypeptide or a fragment thereof comprising a carbohydrate recognition domain (CRD), wherein the targeting molecule is incorporated into the outer surface of the liposome. Provided herein are targeted nanoparticle compositions and methods for the diagnosis, treatment or prevention of an infectious disease using same.

### PRIOR RELATED APPLICATION

This application claims the benefit of and priority to U.S. Provisional Application No. 62/237,687, filed on Aug. 27, 2021, which is hereby incorporated by reference in its entirety.

### STATEMENT AS TO RIGHTS TO INVENTIONS MADE UNDER FEDERALLY SPONSORED RESEARCH AND DEVELOPMENT

This invention was made with government support under Agreement No. R21AI148890 awarded by the NIH. The Government has certain rights in the invention.

### BACKGROUND

Infectious diseases continue to burden populations around the world. Both naturally occurring and engineered biological threats hold increasing potential to cause disease, disability, and death. While some infectious diseases are merely disabling, others are life threatening. Despite advances in the understanding of the pathology of infections, current methods for diagnosing and treating a number of fungal, viral and bacterial infections, to name a few; are deficient.

### SUMMARY

Provided herein are targeted nanoparticles, for example, liposomes, for the diagnosis, treatment or prevention of an infectious disease. In some embodiments, the liposomes comprise a targeting molecule that binds a target antigen expressed by a pathogen, wherein the targeting molecule is a C-Type Lectin polypeptide or a fragment thereof comprising a carbohydrate recognition domain (CRD), wherein the targeting molecule is incorporated into the outer surface of the liposome. In some embodiments, the liposome does not comprise an antipathogenic agent, for example, an antipathogenic agent (e.g., a compound or drug) encapsulated in the liposome. In some embodiments, the liposome comprises an antipathogenic agent (e.g., a compound or drug) encapsulated in the liposome.

In some liposomes, the targeting molecule is a Dendritic Cell-Specific Intercellular adhesion molecule-3-Grabbing Non-integrin (DC-SIGN) polypeptide or a fragment thereof comprising a carbohydrate recognition domain (CRD), and wherein the targeting molecule is incorporated into the outer surface of the liposome. In some liposomes, the fragment comprises a CRD (SEQ ID NO: 1) and one or more neck regions of DC-SIGN selected from the group consisting of (NR1) SEQ ID NO: 2. (NR2) SEQ ID NO: 3, (NR3) SEQ ID NO: 4, (NR4) SEQ ID NO: 5, (NR5) SEQ ID NO: 6, (NR6) SEQ ID NO: 7, (NR7) SEQ ID NO: 8 and (NR8) SEQ ID NO: 9. Some liposomes comprise two or more, three or more, or four or more neck regions of DC-SIGN selected from the group consisting of (NR1) SEQ ID NO: 2. (NR2) SEQ ID NO: 3, (NR3) SEQ ID NO: 4, (NR4) SEQ ID NO: 5, (NR5) SEQ ID NO: 6, (NR6) SEQ ID NO: 7, (NR7) SEQ ID NO: 8 and (NR8) SEQ ID NO: 9. In some liposomes, the fragment comprises SEQ ID NO: 1, SEQ ID NO: 8 and SEQ ID NO: 9. In some liposomes, the fragment comprises an amino acid sequence having at least 90% identity to SEQ ID NO: 10 or SEQ ID NO: 40. In some liposomes, the fragment comprises SEQ ID NO: 1, SEQ ID NO: 2 and SEQ ID NO: 3. In some liposomes, the fragment comprises an amino acid sequence having at least 90% identity to SEQ ID NO: 12 or SEQ ID NO: 41.

In some embodiments, the liposome comprises an antipathogenic agent, wherein the antipathogenic agent is encapsulated in the liposome. In some embodiments, the antipathogenic agent is an antiviral agent, an antifungal agent, an antibacterial agent, an antiprotozoan agent or an antihelminthic agent.

In some embodiments, the target antigen is on a pathogen. In some embodiments, the target antigen is on the cell wall of a pathogenic cell or is an antigen in an exopolysaccharide matrix associated with the pathogenic cell. In some embodiments, the pathogen is a fungus, a virus, bacteria, an animal pathogen or a protozoan. In some embodiments, the fungus is selected from the group consisting of Aspergillus fumigatus, Candida albicans and Cryptococcus neoformans. In some embodiments, the virus is a coronavirus. In some embodiments, the coronavirus is severe acute respiratory syndrome-coronavirus 2 (SARS-COV-2) virus. In some embodiments, the DC-SIGN targeting molecule, or a fragment thereof comprising a CRD, binds to mannans on a spike protein of the SARS-COV2 virus. In some embodiments, when a DC-SIGN targeted nanoparticle described herein binds to a target antigen on a viral particle, the viral particle or a population of viral particles can be sequestered by the liposomes, thus acting as an antiviral agent without incorporation or encapsulation of an antiviral agent (e.g., a drug) in the liposome.

In some embodiments, the antipathogenic agent is an antifungal agent. In some embodiments, the antifungal agent is a polyene, azole or echinocandin antifungal agent. In some embodiments, the polyene antifungal agent is amphotericin B (AmB).

In some liposomes, the DC-SIGN polypeptide, or a fragment thereof comprising a CRD, is conjugated to a lipid or a pegylated lipid. In some liposomes, the concentration of the antifungal drug is reduced as compared to the concentration of the antifungal drug encapsulated in a liposome that does not comprise a DC-SIGN polypeptide, or a fragment thereof comprising a CRD, incorporated into the outer surface of the liposome. In some embodiments, the liposome has decreased affinity for and/or is less toxic to an animal cell as compared to a liposome that does not comprise a DC-SIGN polypeptide, or a fragment thereof comprising a CRD, incorporated into the outer surface of the liposome.

Also provided is a liposome comprising an antibacterial agent and a targeting molecule that binds a target antigen on a bacterial cell, wherein the targeting molecule is incorporated into the outer surface of the liposome and the antibacterial agent is encapsulated in the liposome. In some embodiments, the targeting molecule is a C-lectin polypeptide or a fragment thereof, for example, a targeting molecule selected from the group consisting of Dectin-1 or a fragment thereof, Dectin-2 or a fragment thereof, Dectin-3 or a fragment thereof, and DC-SIGN or a fragment thereof. In some embodiments, the targeting molecule comprises an amino acid sequence having at least 90% identity to SEQ ID NO: 20, SEQ ID NO: 21, SEQ ID NO: 22, SEQ ID NO: 40 or SEQ ID NO: 41. In some embodiments, the bacterial cell is a mycobacterial cell. In some embodiments, the mycobacterial cell is a Mycobacterium tuberculosis cell, a Mycobacterium avium cell or a Mycobacterium ulcerans cell. In some embodiments, the antibacterial agent is an antibiotic. In some embodiments, the antibacterial agent is selected from the group consisting of isoniazid, a rifamycin, rifapentine, rifabutin, pyrazinamide, and ethambutol. In some embodiments, the liposome or a plurality of liposomes comprising an antibacterial agent and a targeting molecule that binds a target antigen on a bacterial cell can target a population of any of the bacterial cells described herein, in vivo, ex vivo or in vitro. In some embodiments, any of the liposomes or pluralities of liposomes comprising an antibacterial agent can be used in any of the methods provided herein to treat a bacterial infection, for example a mycobacterial infection.

Also provided is a plurality of any of the liposomes described herein.

Further provided is a liposome comprising (a) a DC-SIGN targeting molecule, or a fragment thereof comprising a CRD, wherein the targeting molecule or a fragment thereof that binds a target antigen expressed by a pathogen; and (b) a signal-generating molecule, wherein the targeting molecule is incorporated into the outer surface of the liposome and the signal-generating molecule generates a signal when the targeting molecule binds the target antigen.

In some embodiments, the signal-generating molecule is linked to the targeting molecule. In some embodiments, the signal-generating molecule is incorporated into or attached to the outer surface of the liposome. In some embodiments, the signal-generating molecule is a fluorescent dye or fluorescent polypeptide. In some embodiments, the targeting molecule is linked to the C-terminal and/or an N-terminal fragment of a fluorescent protein. Also provided is a plurality of any of the liposomes described herein that comprises a signal-generating molecule.

Also provided is a pharmaceutical composition comprising any of the plurality of liposomes described herein.

Further provided is a method of treating or preventing an infection in a subject comprising administering to the subject having an infection or at risk of developing an infection an effective amount of a plurality of liposomes or a pharmaceutical composition described herein. In some methods, the infection is a viral infection, a fungal infection, or a bacterial infection. In some methods, the fungal infection is an Aspergillus infection, a Cryptococcus infection, a Candida infection or a Trichophyton infection.

In some methods, the viral infection is a coronavirus. In some methods, the coronavirus is SARS-COV2 virus. In some methods, the subject is immunocompromised. In some methods, the subject has pneumonia, asthma, COPD, cystic fibrosis, tuberculosis, emphysema or sarcoidosis. In some methods, the liposomes are administered topically, intranasally, systemically or via inhalation. In some methods, a second therapeutic agent or therapy is administered to the subject. In some methods, the second therapy is surgery. In some methods, the second therapeutic agent is a second antiviral agent, a second antibacterial agent or a second antifungal agent.

Also provided is a method of making a plurality of liposomes comprising an antipathogenic agent and a DC-SIGN targeting molecule or a fragment thereof comprising a CRD, wherein the targeting molecule is incorporated into the outer surface of each liposome and the antipathogenic agent is encapsulated in each liposome, the method comprising the steps of: (a) dissolving the antipathogenic agent in solvent for about 10 minutes to about 30 minutes, at about 60° C.: (b) encapsulating the antipathogenic agent into each liposome by mixing a plurality of liposomes in suspension with the antipathogenic/solvent solution of step a), for about 3 to about 5 hours, at about 60° C. or 37° C. for about 24-120 hours; and (c) incorporating the targeting molecule into the outer surface of each liposome by contacting the liposomes comprising the encapsulated antipathogenic agent with the targeting molecule, for about 45 minutes to about 90 minutes, at 60° C. In some methods the targeting molecule is conjugated to a lipid. In some methods, the lipid is a pegylated lipid. In some embodiments, the method further comprises storing the liposomes comprising an antipathogenic agent and a targeting molecule in a renaturation buffer comprising arginine. In some methods, the antipathogenic agent is an antiviral agent, an antibacterial agent or an antipathogenic agent. In some methods, the antipathogenic agent is an antifungal, wherein the antifungal is a polyene, an azole or an echinocandin antifungal agent. In some methods, the polyene antifungal agent is amphotericin B.

Also provided is a method for detecting an infection in a subject or a sample from a subject comprising: (a) contacting the subject or a sample from the subject with a plurality of any of the targeted liposomes comprising a signaling molecule described herein: (b) detecting a signal, wherein a signal indicates the presence of an infection. In some methods, the targeting molecule is linked to a fluorescent protein, an antibody or a fragment thereof, or an enzyme. In some methods, the signal is directly or indirectly detected. In some methods, the infection is a viral infection, a fungal infection or a bacterial infection.

Further provided is a composition comprising: (a) a DC-SIGN polypeptide or a fragment thereof, wherein the fragment comprises the CRD domain of DC-SIGN; and (b) a renaturation buffer. In some embodiments, the renaturation buffer comprises between about 0.5M L-arginine and 1.5M L-arginine. In some embodiments, the fragment comprises a polypeptide sequence comprising SEQ ID NO: 1. In some embodiments, the fragment comprises SEQ ID NO: 1 and one or more neck regions of DC-SIGN selected from the group consisting of (NR1) SEQ ID NO: 2, (NR2) SEQ ID NO: 3, (NR3) SEQ ID NO: 4, (NR4) SEQ ID NO: 5, (NR5) SEQ ID NO: 6, (NR6) SEQ ID NO: 7, (NR7) SEQ ID NO: 8 and (NR8) SEQ ID NO: 9. In some embodiments, the fragment comprises SEQ ID NO: 1, SEQ ID NO: 8 and SEQ ID NO: 9. In some embodiments, the fragment comprises SEQ ID NO: 10, which is encoded by SEQ ID NO: 11. In some embodiments, the fragment comprises SEQ ID NO: 1, SEQ ID NO: 2 and SEQ ID NO: 3. In some embodiments, the fragment comprises SEQ ID NO: 12, which is encoded by SEQ ID NO: 13.

Also provided is a polypeptide comprising an amino acid sequence having at least 90% identity to SEQ ID NO: 10, SEQ ID NO: 12, SEQ ID NO: 40 or SEQ ID NO: 41. In any of the compositions or polypeptides described herein, the DC-SIGN polypeptide or a fragment thereof comprises a detectable label.

In some embodiments, the detectable label is a fluorescent label.

Also provided is a kit comprising any of the compositions, liposomes or plurality of liposomes described herein.

Also provided is a composition comprising any of the liposomes or plurality of liposomes described herein.

### DESCRIPTION OF THE DRAWINGS

The present application includes the following figures. The figures are intended to illustrate certain embodiments and/or features of the compositions and methods and to supplement any description(s) of the compositions and methods. The figures do not limit the scope of the compositions and methods, unless the written description expressly indicates that such is the case.

FIGS. 1A and 1B show that recombinant human DC-SIGN isoforms were expressed and purified and used to target drug loaded liposomes to fungal glycans. (A) The domain structure of human DC-SIGN and two truncated recombinant isoforms DCS78 and DCS12 are shown. Full length native human DC-SIGN is composed of a carbohydrate recognition domain (CRD), eight neck repeats, a membrane spanning domain (MSD), a signaling domain (SD) and a transit peptide domain (TD). Two truncated recombinant isoforms of human DC-SIGN, DCS78 and DCS12, each with two neck repeats fused to the CRD, were expressed and purified. They were then used to target drug loaded liposomes. (B) DCS12 and DCS78 monomers, homodimers, and/or homotetramers (green globular structures) were expected to bind to glycans (e.g., red sugar moieties) in the membranes and exopolysaccharide matrices of fungal cells. DCS12 and DCS78 were coupled to the lipid carrier DSPE-PEG-NHS via the reactive NHS group DHPE-Rhodamine (red star), and DCS12-PEG-DSPE or DCS78-PEG-DSPE and were inserted into the liposomal membrane of amphotericin B loaded liposomes, AmB-LLs, via their lipid moieties, DHPE and DSPE, to make red fluorescent DC-SIGN targeted liposomes. The engineered DC-SIGN isoforms each contain one CRD and two neck repeats. Each recombinant monomer is free to float in the membrane and form multimers with enhanced binding. Each liposome contains approximately 3,000 rhodamine molecules, 1,500 monomers of one DC-SIGN isoform, and 16,500 AmB molecules.

FIGS. 2A-2C show that DCS12-AmB-LLs and DCS78-AmB-LLs bound more efficiently to the exopolysaccharide matrices of three highly divergent fungal pathogens grown in vitro than control liposomes. (A) C. albicans (10× magnification). (B) A. fumigatus (10× magnification). (C) C. neoformans (20× magnification). Bright field images of fungal cells were fused with red fluorescent images of rhodamine red liposomes. 100- or 200-micron size bars are shown. DCS12-AmB-LLs bound to all three fungal species more efficiently than DCS78-AmB-LLs. Negligible binding was detected by BSA coated BSA-AmB-LLs or uncoated AmB-LLs. The area of red fluorescent liposome binding was quantified and shown in the scatter bar plots on the right for all three species.

FIG. 3 shows that DCS12-AmB-LLs delivering low concentrations of AmB inhibited or killed in vitro grown C. albicans more efficiently than control liposomes. Wells of a microtiter plate were seeded with 4,000 C. albicans cells per well. Cells were grown to late germling and early hyphal stages and treated with liposomes delivering 0.1. 0.05 and 0.025 μM AmB. Liposomes were removed and fresh media added. The cells were incubated again and then assayed for metabolic activity by incubating with CellTiter-Blue reagent. Scatter bar plots showing the log₁₀ of Relative Fluorescent Units (RFUs) indicating the relative inhibition and/or killing of fungal cells by different liposome preparations. Each assay is from 6 replicate wells on the plate. The fold-difference in RFU values and P values for the comparison of DCS12-AmB-LLs to AmB-LLs are shown for the assays performed for 0.05 and 0.025 μM AmB. Replicate assays and assays using higher concentrations of AmB are shown in FIG. 7.

FIGS. 4A-4B show that DCS12-AmB-LLs were significantly more effective at reducing the number of C. albicans cells in the kidneys than AmB-LLs. Neutropenic mice infected with C albicans cells were treated with liposomes delivering 0).2 mg/kg AmB. (A) A scatter bar plot compares the average number of CFUs per kidney pair for the three treatment groups. Control buffer, AmB-LLs, and DEC2-AmB-LLs. Six mice were in each treatment group and each mouse is represented by one data point. (B) The Relative Quantity (RQ) of C. albicans rDNA intergenic spacer (IGS) was determined by qPCR on parallel samples of kidney homogenates from the same mice. Three replicates qPCR reactions were run on each sample. Standard errors, fold differences and P values between some pairs of samples are indicated. A replicate experiment is shown in FIG. 8.

FIGS. 5A-5C show the Amino acid sequences of human DC-SIGN, DCS12 and DCS78. (A) Annotated amino acid (a.a.) sequence of full-length human DC-SIGN (CD209 Q9NNX6.1 404 a.a.) (SEQ ID NO: 16). (B) Annotated amino acid sequence of recombinant DCS12 made in E. coli which has a total of 221 amino acids, MW 24,855 g/mole (24.9 kDa) (SEQ ID NO: 12). From Protparam expasy. Theoretical pI=5.90. 2.291 OD/mg/mL A280 with cystine residues reduced. Aliphatic index 55.25 and hydrophobicity-0.767. Its instability index is 47.74. (C) Annotated amino acid sequence of recombinant DCS78 made in E. coli which has a total of 223 amino acids (SEQ ID NO: 10), MW 25,191.96 g/mole (25.2 kDa). From ProtParam Expasy. Theoretical pI=5.91. 2.26 OD/mg/mL A280 with cystine residues reduced. Aliphatic index 52.56 and hydrophobicity-0.846. Its instability index is 50.91.

FIG. 6 shows SDS PAGE analysis of affinity purified DCSIGN12 (DCS12) and DCSIGN78 (DCS78), before and after coupling to DSPE-PEG-NHS. Samples were resolved on a 12% polyacrylamide gel and stained with Coomassie Blue. Molecular weight markers visible before Coomassie staining were tagged by poking the gel with a needle with carbon particle. Their sizes are indicated in kilo-Daltons (kDa). PEG is extremely hydrophilic and reduces Coomassie staining.

FIGS. 7A-7B show inhibition of the metabolic activity of in vitro grown C. albicans by DCS12-AmB-LLs and AmB-LLs delivering AmB concentrations of 0.4. 0.2. 0.1, and 0.05 μM. (A) Wells of a microtiter plate were seeded with 4,000 C. albicans cells per well. Cells were grown to late germling and early hyphal stages and treated for 60 min with liposomes delivering 0.1. 0.05 and 0.025 μM AmB. For experimental details see legend to FIG. 3 and Examples. (B) This experiment was conducted and analyzed by methods similar to those described in FIGS. 3 and 7A, except those plates were seeded with 40.000 cells per microtiter well instead of 10.000 cells/well, and cells were only grown for 3 hours after liposomes were removed, and just before adding CTB reagent. Six replicate wells were in each concentration tested. Standard errors, fold differences and P values between some pairs of samples are indicated.

FIGS. 8A-8B show that DCS12-AmB-LLs were more effective at reducing the number of C albicans cells in the kidneys than AmB-LLs. Neutropenic mice infected with (. albicans cells were treated with liposomes delivering 0.2 mg/kg AmB. See conditions in the legend to FIG. 4 and Examples. (A) A bar graph compares the average number of CFUs per kidney pair for the three treatment groups. Control buffer and AmB-LLs or DEC2-AmB-LLs. (B) The Relative Quantity (RQ) of C. albicans rDNA intergenic spacer (IGS) was determined by qPCR on parallel samples of kidney homogenates from the same mice. Three replicates qPCR reactions were run on each sample. Six mice were in each treatment group. Standard errors, fold differences and P values between some pairs of samples are indicated.

FIG. 9A shows that DCS 12 is an isoform of DC-SIGN. DCS12 monomers and tetramers (green globular) bind to N-linked mannans (e.g., red sugar) expressed in ectodomain of a Sars CoV2 Spike trimer (red forked structure). DC-SIGN is coupled to the lipid carrier DSPE-PEG (green globular structure). DCS12-PEG-DSPE was inserted into the liposomal membrane via its DSPE lipid moiety. The engineered DCS12 isoform illustrated contains one CDR and two neck repeats. Each DCS12-PEG-DSPE monomer is free to float in the membrane and form multimers/tetramers with enhanced binding. Each liposome contains approximately 1.500 DC-SIGN monomers. The virus particle has ˜100 Spike trimers. Hence, the liposome could bind the virus with great avidity.

FIG. 9B is an illustration of how several DC-SIGN liposomes could bind, surround, and sequester a single SARS COV-2 virion, inhibiting its replication cycle.

FIG. 10A shows a model of Dectin-1-coated rhodamine B tagged liposomes binding to pathogenic mycobacteria. Targeted liposomes, also referred to as dectisomes, such as, for example, those targeted by Dectin-1, are designed to bring antibacterial drugs in close proximity to pathogenic mycobacteria and concentrate drug away from host cells (right side). Untargeted drugs are just as readily associated with host cells as the pathogen (left side).

FIG. 10B shows the design of a 100 nm-diameter pegylated liposome. Dectin-1 (DEC1) and rhodamine B, coupled to lipid carriers DSPE-PEG and DHPE, are inserted via these two lipid moieties into the liposomal membrane to make DEC1-Rhod-Ls. Dectin-1 monomers float together in the liposomal membrane to form dimers that bind mycobacterial beta-glucan oligomers (orange sugar moieties on cells). Each DEC1-Rhod-L contains approximately 1,500 DEC1 monomers, 3,000 rhodamine molecules. In the future, we would construct each liposome to contain several thousand antibacterial drug molecules. FIG. 11A is an overview of Dectin-1 coated liposomes, DEC1-Rhod-Ls, binding to M. avium cells and cell clusters taken at 20× magnification.

FIGS. 11B-11F are representative images of liposome binding to cell clusters taken at 63× magnification under oil immersion using epifluorescence microscopy. (B) DEC1-Rhod-Ls, (C) DEC2-Rhod-Ls. (D) DCS12-Rhod-Ls, (E) Rhod-Ls, (F) BSA-Rhod-Ls. These images were enhanced in the green and red channels for better viewing of GFP tagged cells and rhodamine B red fluorescent liposomes. Size bars in microns indicate the degree of magnification. A replicate experiment is shown in FIG. 13.

FIG. 12A shows the efficiency and specificity of targeted liposome binding to M. avium. Quantification of liposome binding was conducted. A scatter bar plot made from eight random unenhanced images taken at 63× magnification (i.e., representative images in FIG. 11) shows the area of red fluorescent DEC1-Rhod-Ls, DEC2-Rhod-Ls, and DCS12-Rhod-Ls binding as compared to untargeted Rhod-Ls and BSA-Rhod-Ls. Only M. avium cell clusters containing 10 or more cells were assayed for fluorescent liposome binding. The bars are shown in Log₁₀ scale to better illustrate the wide dynamic range of all data points that distinguished targeted from untargeted liposome binding. N=8 for each bar.

FIG. 12B shows the Specificity of DEC1-Rhod-L and DEC2-Rhod-L binding to their cognate oligoglycans. A scatter bar plot shows the relative inhibition of liposome binding to M. avium cell clusters by the oligo-glucan laminarin and yeast oligo-mannan demonstrating the specificity of each type of liposome by their respective oligoglycan ligands. N=10 images for each treatment. Standard errors for each value are indicated. The fold differences and P or PMW values are shown for the most relevant comparisons.

FIGS. 13A-13E show that Dectin-1, Dectin-2, and DCS12-targeted liposomes bound efficiently to M. ulcerans, while control liposomes did not. Shown are representative images of liposome binding to M. ulcerans cells taken at 63× magnification under oil immersion using epifluorescence microscopy. (A) DEC1-Rhod-Ls, (B) DEC2-Rhod-Ls. (C) DCS12-Rhod-L, (D) Rhod-Ls, and (E) BSA-Rhod-Ls. These images were enhanced for better viewing of CW stained cells and rhodamine B red fluorescent liposomes.

FIG. 13F is a scatter bar plot made from eight random unenhanced images, taken at 63λ, showing the area of red fluorescent liposome binding to cell clusters (Log10 scale). Standard errors for each value and the fold-differences and PMW values comparing the area of DectiSome binding to untargeted Rhod-Ls are shown. A size bar in microns indicates the degree of magnification, which is 4-fold greater than that in FIG. 11.

FIGS. 14A-14E show that Dectin-1, Dectin-2, and DCS12-targeted liposomes bound efficiently to M. tuberculosis pellicle cell clusters, while control liposomes did not. Representative images of liposome binding to M. tuberculosis cells were taken at 63× magnification using epifluorescence microscopy. (A) DEC1-Rhod-Ls, (B) DEC2-Rhod-Ls. (C) DCS12-Rhod-L, (D) Rhod-Ls, and (E) BSA-Rhod-Ls. These images were enhanced for better viewing of the bright field cell images and the red fluorescent liposomes.

FIG. 14F is a scatter bar plot made from 10 random unenhanced images taken at 63X showing the area of red fluorescent liposome binding to cell clusters (Log10) scale). Standard errors for each value and the fold-differences and P_{MW} values comparisons of the area of targeted liposome binding to untargeted Rhod-Ls are shown. A size bar in microns indicates the degree of magnification.

FIG. 15 shows that DCS12-targeted liposomes (DCS12-AmB-LLs) bind to T. gondii strain ME49 growing in infected cultures of human foreskin fibroblasts (HFFs). Dectin-1 coated liposomes, (DEC1-AmB-LLs) did not bind to T. gondii strain ME49 growing in infected cultures of human foreskin fibroblasts (HFFs).

### DETAILED DESCRIPTION

The following description recites various aspects and embodiments of the present compositions and methods. No particular embodiment is intended to define the scope of the compositions and methods. Rather, the embodiments merely provide non-limiting examples that are at least included within the scope of the disclosed compositions and methods. The description is to be read from the perspective of one of ordinary skill in the art: therefore, information well known to the skilled artisan is not necessarily included.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations were interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original): see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

### Nanoparticles

Provided herein are nanoparticles for the diagnosis, treatment or prevention of an infectious disease. As used throughout, nanoparticles can be, but are not limited to, lipid nanoparticles, for example, liposomes or non-liposomal lipid nanoparticles (for example, lipid nanoparticles with a non-aqueous core (LNPs)), dendrimers, polymeric micelles, nanocapsules or nanospheres, to name a few. For example, provided herein is a nanoparticle, for example, a liposome, comprising a targeting molecule that binds a target antigen expressed by a pathogen, wherein the targeting molecule is a C-Type Lectin polypeptide or a fragment thereof comprising a carbohydrate recognition domain (CRD), wherein the targeting molecule is incorporated into the outer surface of the liposome. Exemplary human C-Type Lectin polypeptides from which targeting molecules can be derived are set forth below in Table 1 with the corresponding UniProt Nos. for their amino acid sequences. In some embodiments, the nanoparticle, for example, a liposome, comprises an antipathogenic agent (e.g., an antiviral, antifungal, an antiparasitic, or an antiprotozoal drug) encapsulated in the liposome. In some embodiments, the antipathogenic agent encapsulated in the liposome is not an antifungal agent. In some embodiments, the antipathogenic agent encapsulated in the liposome is not an antiviral agent. In some embodiments, the antipathogenic agent encapsulated in the liposome is not an antiparasitic agent. In some embodiments, the antipathogenic agent encapsulated in the liposome is not an antiprotozoal agent.

In some embodiments, the liposome does not comprise an antipathogenic agent, (e.g., an antiviral, antifungal, an antiparasitic, or an antiprotozoal drug) encapsulated in the liposome. It is understood that any of the liposomes provided herein can have antipathogenic or anti-infective activity without comprising an encapsulated antipathogenic compound or drug (e.g., an antiviral, antifungal, an antiparasitic, or an antiprotozoal drug). In some embodiments, the liposome itself is an antifungal agent, an antiviral agent, an antibacterial agent, an antiprotozoal agent or an antiparasitic agent.

Also provided are liposomes that do not comprise an antipathogenic agent (e.g., an antiviral, antifungal, an antiparasitic, or an antiprotozoal drug), wherein the targeting molecule does not comprise an amino acid sequence encoding Dectin-1, Dectin-2 or Dectin-3 or a fragment of Dectin-1, Dectin-2 or Dectin-3, from any species.

TABLE 1
C-type Lectins

| Gene | | | Human |
|---|---|---|---|
| Name | Descriptive Name | Protein Names | UniProt No. |
| CD209 | Dendritic Cell-Specific | DC-SIGN | Q9NNX6 |
| | ICAM-3-Grabbing Non- | | |
| | Integrin 1 | | |
| CLEC4M | Dendritic Cell-Specific | DC-SIGNR | Q9H2X3 |
| | ICAM-3-Grabbing Non- | | |
| | Integrin 2 | | |
| CLEC7A | C-Type Lectin Domain | Dectin-1, CD369 | Q9BXN2 |
| | Containing 7A | | |
| CLEC6A | C-Type Lectin Domain | Dectin-2 | Q6EIG7 |
| | Containing 6A | | |
| CLEC4D | C-Type Lectin Domain | Dectin-3, MCL, | Q8WXI8 |
| | Family 4 Member D | CD368 | |
| MBL2 | Mannose Binding Lectin | Mannose Binding | P11226 |
| | 2 | Protein, MR | |
| CLEC4E | C-Type Lectin Domain | Mincle | Q9ULY5 |
| | Family 4 Member E | | |
| SFTPA1 | Surfactant Protein A | SP-A | Q8IWL2 |
| SFTPD | Surfactant Protein D | SP-D | P35247 |
| ITGAM | Integrin Subunit Alpha | CR-3, CD11b | P11215 |
| | M | | |
| CD207 | C-Type Lectin Domain | Langerin, CD207 | Q9UJ71 |
| | Family 4 Member K | | |
| CLEC1A | C-Type Lectin Domain | MelLec, CLEC1 | Q9UJ71 |
| | Family 1 Member A | | |
| OLR1 | Oxidized Low Density | LOX-1, CLEC8A, | P78380 |
| | Lipoprotein Receptor 1 | LOXIN | |

### DC-SIGN

In some liposomes, the targeting molecule is a Dendritic Cell-Specific Intercellular adhesion molecule-3-Grabbing Non-integrin (DC-SIGN) polypeptide or a fragment thereof comprising a carbohydrate recognition domain (CRD), and wherein the targeting molecule is incorporated into the outer surface of the liposome.

Dendritic Cell-Specific Intercellular adhesion molecule-3-Grabbing Non-integrin (DC-SIGN or CD209) is a type II membrane protein, with a single CRD, expressed on the surface of immature dendritic cells (DCs), involved in initiation of the primary immune response. Human DC-SIGN is a 404 amino acid residue polypeptide that comprises an N-terminal cytoplasmic tail, a transmembrane domain, and an extracellular domain comprising eight neck regions (NR1—NR8) and a carbohydrate recognition domain (CRD). The full-length sequence of DC-SIGN is set forth under UniProtKB No. Q9NNX6, and set forth herein as SEQ ID NO: 16. Amino acids 1-70 of SEQ ID NO: 16 comprise the signal sequence and transmembrane domain of DC-SIGN (SEQ ID NO: 17). It is understood that polypeptide sequences comprising a polypeptide fragment of DC-SIGN that does not include the signal sequence and/or transmembrane domain are also provided herein. For example, fragment of amino acids 71-404 are provided herein, including fragments with N-terminal and/or C-terminal deletions of 1, 2, 3, 4, 5, 6, 7, 8, 9, 10, 11, 12, 13, 14, 15, 16, 17, 18, 19, 20 or more amino acids.

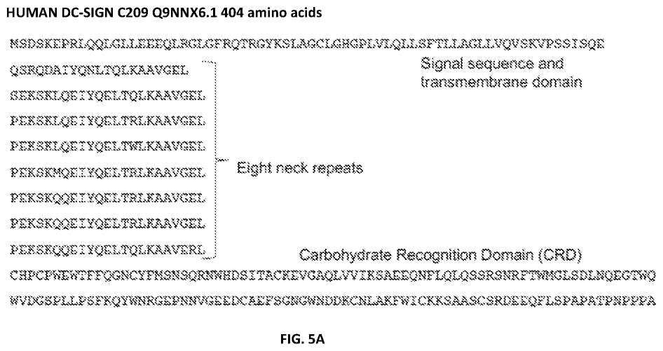

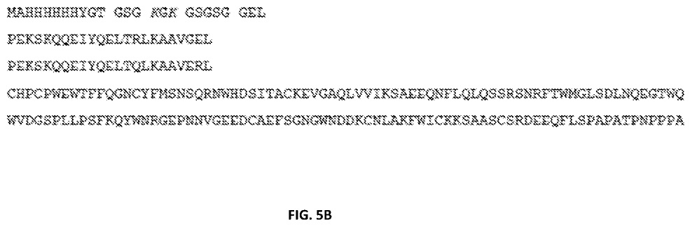

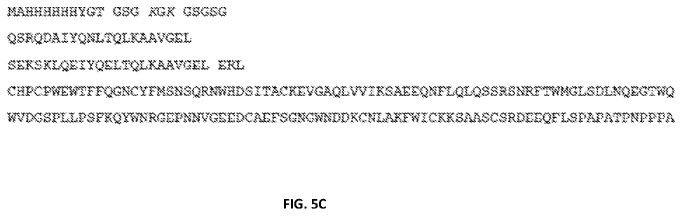

In some liposomes, the DC-SIGN polypeptide is not a full-length DC-SIGN polypeptide In some liposomes, the fragment comprises a CRD (SEQ ID NO: 1) and one or more neck regions of DC-SIGN selected from the group consisting of (NR1) SEQ ID NO: 2, (NR2) SEQ ID NO: 3, (NR3) SEQ ID NO: 4, (NR4) SEQ ID NO: 5, (NR5) SEQ ID NO: 6, (NR6) SEQ ID NO: 7, (NR7) SEQ ID NO: 8 and (NR8) SEQ ID NO: 9. See also, FIG. 5A. It is understood that the CDR and one or more neck regions of DC-SIGN can be joined with or without a linker having about 2, 3, 4, 5, 6, 7, 8, 9, 10 or more amino acids. Examples of linkers include but are not limited to GSGₙ wherein n is an integer, and GSGSG (SEQ ID NO: 18). Any of the DC-SIGN polypeptides provided herein can also be linked to a tag, for example, a histidine tag, as described below.

In some liposomes, the fragment comprises, consists essentially of, or consists of, a DC-SIGN CRD (SEQ ID NO: 1), NR7 (SEQ ID NO: 8) and NR8 (SEQ ID NO: 9). An example of this construct is described herein as DSC78. In some liposomes, the fragment comprises, consists essentially of, or consists of SEQ ID NO: 10 (DCS78 construct) set forth below. N terminal amino acid and (His) 6 (HHHHHH) (SEQ ID NO: 19) affinity tag from a pET-45B+vector is boxed. Gly Ser (GS) flexible linker residues and reactive lys (K) residues for linking to lipid carrier are in bold with lysines in italic. Human DC-SIGN amino acid residues appear in plain text and includes 2×23 a.a. neck repeats 7 and 8 and all 153 a.a. of CDR (total of 201 a.a. from Hs DC-SIGN), ending in a C-terminal Ala residue (A) in bold, the codon for which was used to put stop codons and PacI site in frame. It is understood that fragments of SEQ ID NO: 10 that do not comprise the His tag (boxed), the linker sequence (bold) and/or the C-terminal Ala residue of SEQ ID NO: 10 are also provided. SEQ ID NO: 40 is an exemplary sequence comprising a DC-SIGN CRD (SEQ ID NO: 1), NR7 (SEQ ID NO: 8) and NR8 (SEQ ID NO: 9). Fragments of SEQ ID NO: 40 are also provided.

(SEQ ID NO: 10)

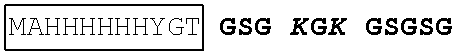

GELPEKSKQQEIYQELTRLKAAVGELPEKSKQQEIYQELTQLKAAVERLCHPCPWEW
TFFQGNCYFMSNSQRNWHDSITACKEVGAQLVVIKSAEEQNFLQLQSSRSNRFTWM
GLSDLNQEGTWQWVDGSPLLPSFKQYWNRGEPNNVGEEDCAEFSGNGWNDDKCN
LAKFWICKKSAASCSRDEEQFLSPAPATPNPPPA

In some liposomes, the fragment comprises, consists essentially of, or consists of, a DC-SIGN (CRD) SEQ ID NO: 1, NR1 (SEQ ID NO: 2) and NR2 (SEQ ID NO: 3). An example of this construct is described herein as DCS12. In some liposomes, the fragment comprises, consists essentially of, or consists of, SEQ ID NO: 12 (DSC12 construct) as set forth below. An N terminal amino acid and (His) 6 (HHHHHH) affinity tag from a pET-45B+vector is boxed. Gly Ser (GS) flexible linker residues and reactive lys (K) residues for linking to lipid carrier are in bold with lysines in italic. Human DC-SIGN amino acid residues appear in plain text and includes 2×23 a.a. neck repeats 1 and 2 (underlined) and all 153 a.a. of CDR (total of 201 a.a. from Hs DC-SIGN splice variant 2d (Serrano Gomez et al. “Structural Requirements for Multimerization of the Pathogen Receptor Dendritic Cell-specific ICAM3-grabbing Non-integrin (CD209) on the Cell Surface,” Journal of Biological Chemistry 283, 3889-3903 (2008)), ending in a C-terminal Ala residue (A) in bold, the codon for which was used to put stop codons and PacI site in frame. It is understood that fragments of SEQ ID NO: 12 that do not comprise the His tag (boxed) the linker sequence (bold) and/or the C-terminal Ala residue are also provided. SEQ ID NO: 41 is another exemplary sequence comprising a DC-SIGN (CRD) SEQ ID NO: 1, NR1 (SEQ ID NO: 2) and NR2 (SEQ ID NO: 3). Fragments of SEQ ID NO: 41 are also provided.

(SEQ ID NO: 12)

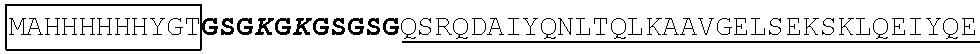

LTQLKAAVERLCHPCPWEWTFFQGNCYFMSNSQRNWHDSITACKEVGAQLVVIKSA
EEQNFLQLQSSRSNRFTWMGLSDLNQEGTWQWVDGSPLLPSFKQYWNRGEPNNVG
EEDCAEFSGNGWNDDKCNLAKFWICKKSAASCSRDEEQFLSPAPATPNPPPA

Other examples of DC-SIGN polypeptides include, but are not limited to:
a polypeptide comprising, consisting essentially of, or consisting of a DC-SIGN CRD (SEQ ID NO: 1), (NR1) SEQ ID NO: 2, (NR2) SEQ ID NO: 3, (NR3) SEQ ID NO: 4, (NR4) SEQ ID NO: 5, (NR5) SEQ ID NO: 6, (NR6) SEQ ID NO: 7, (NR7) SEQ ID NO: 8 and (NR8) SEQ ID NO: 9 (See, for example, SEQ ID NO: 14, encoded by SEQ ID NO: 16)
a polypeptide comprising, consisting essentially of, or consisting of a DC-SIGN CRD (SEQ ID NO: 1), (NR1) SEQ ID NO: 2, (NR6) SEQ ID NO: 7, (NR7) SEQ ID NO: 8 and (NR8) SEQ ID NO: 9
polypeptide comprising, consisting essentially of, or consisting of a DC-SIGN CRD (SEQ ID NO: 1), (NR1) SEQ ID NO: 2, (NR2) SEQ ID NO: 3, (NR3) SEQ ID NO: 4, and (NR8) SEQ ID NO: 9
a polypeptide comprising, consisting essentially of, or consisting of a DC-SIGN CRD (SEQ ID NO: 1), (NR1) SEQ ID NO: 2, (NR2) SEQ ID NO: 3, (NR7) SEQ ID NO: 8 and (NR8) SEQ ID NO: 9
a polypeptide comprising, consisting essentially of, or consisting of a DC-SIGN CRD (SEQ ID NO: 1) and (NR1) SEQ ID NO: 2, (NR2)
a polypeptide comprising, consisting essentially of, or consisting of a DC-SIGN CRD (SEQ ID NO: 1), (NR1) SEQ ID NO: 2, (NR2) SEQ ID NO: 3, (NR3) SEQ ID NO: 4, (NR4) SEQ ID NO: 5, (NR5) SEQ ID NO: 6, (NR7) SEQ ID NO: 8 and (NR8) SEQ ID NO: 9
a polypeptide comprising, consisting essentially of, or consisting of a DC-SIGN CRD (SEQ ID NO: 1), (NR1) SEQ ID NO: 2, (NR2) SEQ ID NO: 3, (NR3) SEQ ID NO: 4, (NR4) SEQ ID NO: 5, (NR7) SEQ ID NO: 8 and (NR8) SEQ ID NO: 9
a polypeptide comprising, consisting essentially of, or consisting of a DC-SIGN CRD (SEQ ID NO: 1), (NR1) SEQ ID NO: 2, (NR3) SEQ ID NO: 4, (NR4) SEQ ID NO: 5, (NR7) SEQ ID NO: 8 and (NR8) SEQ ID NO: 9

Other examples of C-type lectin receptors that can be used as targeting molecules include Dectin-1 (CLEC7A, mouse GenBank Accession No.: AAS37670 and human GenBank Accession No.: NP_922938 (SEQ ID NO: 42), Dectin-2 (CLEC6A mouse Genbank Accession No.: NP_064385 and human GenBank Accession No.: Q6EIG7 (SEQ ID NO: 43)), Dectin-3 (CLEC4D mouse GenBank Accession No.: NP_034949 and human UniProt Accession No.: Q8WX18 (SEQ ID NO: 44)) and fragments of Dectin-1, Dectin-2, or Dectin-3. In some liposomes, the Dectin-1, Dectin-2, or Dectin-3 polypeptide is not a full-length Dectin-1, Dectin-2, or Dectin-3 polypeptide. Fragments of Dectin-1, Dectin-2, or Dectin-3 include fragments of SEQ ID NO: 42, SEQ ID NO: 43, and or SEQ ID NO: 44, respectively, with N-terminal and/or C-terminal deletions of 1, 2, 3, 4, 5, 6, 7, 8, 9, 10, 11, 12, 13, 14, 15, 16, 17, 18, 19, 20, 21, 22, 23, 24, 25 or more amino acids.

Exemplary targeting molecules include but are not limited to soluble human Dectin-1, Dectin-2 and Dectin-3 polypeptides comprising SEQ ID NO: 20, 21, and 22, respectively. Other exemplary targeting molecules include but are not limited to soluble mouse Dectin-1, Dectin-2 and Dectin-3 polypeptides comprising SEQ ID NO: 23, 24, or 25, respectively. Fragments of SEQ ID NOs: 20, 21, 22, 23, 24 or 25, for example, fragments comprising a deletion of 1, 2, 3, 4, 5, 6, 7, 8, 9, 10 or more amino acids from the C-terminal and/or N-terminal end of a polypeptide comprising or consisting of SEQ ID NOs: 20, 21, 22, 23, 24 or 25, are also provided and can be used in any of the liposomes, polypeptides or compositions described herein.

Other exemplary constructs that can be used in any of the liposomes described herein are set forth as follows. SEQ ID NO: 26, shown below, is a nucleic acid sequence encoding an exemplary codon optimized soluble mouse Dectin-1 (sDectin-1). A vector pET-45b+sequence of 9 codons is boxed with the start codon underlined. Sites for cloning into pET-45B+KpnI (GGTACC) (SEQ ID NO: 23) and PacI (TTAATTAA) (SEQ ID NO: 21), respectively, are underlined. Codons for Gly Ser (G,S) flexible linker residues are shown in bold and codons for reactive lys (K) residues (AAG) are shown in bold, with lysine codons in italic). The mouse sDectin-1 sequence (CLEC7A, GenBank No. AAS37670.1) is shown in plain text: an Ala codon GCT and stop codons TAA and TTA are underlined, with stop codons in bold. Alternative gene name MmsDectinllyshis. The length of the nucleotide sequence is 604 base pairs, with 597 base pairs encoding a protein of 199 amino acids in length.

(SEQ ID NO: 26)

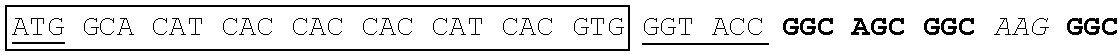

AAG GGC AGC GGC AGC GGT TTT TGG
CGTCACAACAGCGGTCGTAACCCGGAGGAGAAAGACAACTTCCTGAGCCGTAAC
AAGGAGAACCACAAACCGACCGAGAGCAGCCTGGACGAAAAGGTTGCGCCGAG
CAAAGCGAGCCAGACCACCGGTGGCTTCAGCCAACCGTGCCTGCCGAACTGGAT
CATGCACGGCAAGAGCTGCTACCTGTTCAGCTTTAGCGGTAACAGCTGGTATGGC
AGCAAACGTCATTGCAGCCAGCTGGGTGCGCACCTGCTGAAGATCGACAACAGC
AAAGAGTTCGAATTTATTGAGAGCCAGACCAGCAGCCACCGTATCAACGCGTTTT
GGATTGGTCTGAGCCGTAACCAAAGCGAGGGTCCGTGGTTCTGGGAAGATGGCA
GCGCGTTCTTTCCGAACAGCTTTCAAGTGCGTAACACCGCGCCGCAAGAAAGCCT
GCTGCACAACTGCGTTTGGATTCACGGCAGCGAGGTTTACAATCAAATCTGCAAT
ACCAGCAGCTACAGCATCTGCGAGAAG GAA CTG GCT TAA TTA A

SEQ ID NO: 27 is an amino acid sequence encoded by SEQ ID NO: 26. This is a polypeptide comprising a mouse sDectin-1 polypeptide. The N-terminal amino acid sequence and (His) 6 (HHHHHH) (SEQ ID NO: 19) affinity tag is boxed. The Gly Ser (GS) flexible linker residues and reactive lys (K) residues appear in bold with lysines in italic. Mouse sDectin-1 amino acid residues appear in plain text, ending in a C-terminal Ala residue (A) in bold, the codon for which was used to put stop codons and a PacI site in frame. It is understood that, optionally, a stop codon in any of the polypeptide sequences disclosed herein, if not part of the native polypeptide from which the polypeptide is derived, can be removed, to produce a polypeptide that does not include one or more stop codons. The protein comprising the mouse sDectin-1 polypeptide is 199 amino acids in length, with a molecular weight (MW) of 22,389.66 g/mole. The theoretical pI is 7.74. It is understood that any protein described herein comprising an affinity tag, for example, a (His) 6 affinity tag, can be modified to remove the His tag. In some examples, any nucleotide sequence described herein can further comprise a protease cleavage site for post-translational and/or post-purification removal of the affinity tag. In some examples, the soluble mouse Dectin-1 polypeptide comprises amino acids 23-198 of SEQ ID NO: 27.

(SEQ ID NO: 27)

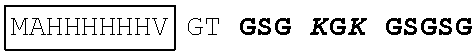

FWRHNSGRNPEEKDSFLSRNKENHKPTESSLDEKVAPSKASQTTGGFSQSCLPNWIM
HGKSCYLFSFSGNSWYGSKRHCSQLGAHLLKIDNSKEFEFIESQTSSHRINAFWIGLSR
NQSEGPWFWEDGSAFFPNSFQVRNAVPQESLLHNCVWIHGSEVYNQICNTSSYSICE
KELA

SEQ ID NO: 28 is a nucleic acid sequence encoding an exemplary codon optimized soluble mouse Dectin-2 (sDectin-2) (SEQ ID NO: 29). The vector pET-45b+sequence of 9 codons is boxed with the start codon underlined. Sites for cloning into pET-45B+KpnI (GGTACC) (SEQ ID NO: 38) and PacI (TTAATTAA) (SEQ ID NO: 39), respectively, are underlined. Codons for Gly Ser (G,S) flexible linker residues appear in bold and the codons for reactive lys (K) residues (AAG) appear in bold, with lysine codons in italic. Codon optimized sDectin-2 from the CLEC6A mouse Dectin 2 gene appears in plain text, with an Ala codon (GCT) and stop codons, TAA and TTA, underlined and stop codons in bold. The alternative gene name is MmsDectin2lyshis. The length of the nucleic acid sequence is 574 base pairs, with 567 base pairs encoding a protein that is 190 amino acids in length. The nucleic acid encoding the codon-optimized mouse sDectin-2 exemplary was cloned into pET-45B+.

(SEQ ID NO: 28)

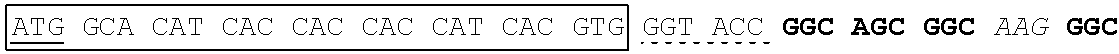

AAG GGC AGC GGC AGC GGT ATA ATG GAT CAA CCC TCA AGA AGG CTA TAT
GAG CTG CAC ACG T?C CAC AGC TCC CTC ACG TGC TTT TCT GAG GGT ACT
ATG GTG TCC GAG AAA ATG TGG GGC TGC TGC CCG AAT CAT TGG AAA TCT
TTT GGT AGC AGC TGT TAT CTG ATC AGC ACC AAA GAG AAC TTC TGG AGT
ACC AGC GAG CAA AAC TGC GTC CAG ATG GGC GCA CAC CTG GTT GTG ATT
AAC ACC GAA GCG GAA CAG AAC TTC ATC ACC CAG CAA TTA AAT GAA AGC
TTG TCT TAC TTC CTG GGT CTG TCG GAT CCG CAG GGC AAC GGC AAG TGG
CAG TGG ATT GAC GAC ACC CCG TTC TCC CAA AAC GTG OGC TTT TGG CAT
CCG CAT GAA CCG AAT CTG CCG GAA GAA CGT TGT GTA AGC ATT GTT TAT
TGG AAT CCA AGC AAG TGG GGT TGG AAC GAC GTT TTT TGT GAT AGC AAG
CAC AAC TCG ATC TGC GAG ATG AAA AAG ATC TAC TTG GCT TAA TTA A

SEQ ID NO 29 is an amino acid sequence encoded by SEQ ID NO: 28. This polypeptide comprises a mouse sDectin-2 protein. The N terminal amino acid and (His) 6 (HHHHHH) (SEQ ID NO: 19) affinity tag from pET-45B+is boxed. The Gly Ser (GS) flexible linker residues and reactive lys (K) residues appear in bold, with lysines in italic. Mouse sDectin-2 amino acid residues appear in plain text ending in a C-terminal Ala residue (A) (bold), the codon for which was used to put stop codons and PacI site in frame. The polypeptide comprising the mouse sDectin-2 that is produced has 189 amino acids, with a MW of 21,699.25 g/mole and a theoretical pI of 6.33. In some examples, the soluble mouse Dectin-2 polypeptide comprises amino acids 23-188 of SEQ ID NO: 29.

(SEQ ID NO: 29)

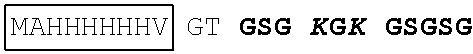

IMDQPSRRLYELHTYHSSLTCFSEGTMVSEKMWGCCPNHWKSFGSSCYLISTKENFW
STSEQNCVQMGAHLVVINTEAEQNFITQQLNESLSYFLGLSDPQGNGKWQWIDDTPF
SQNVRFWHPHEPNLPEERCVSIVYWNPSKWGWNDVFCDSKHNSICEMKKIYLA

SEQ ID NO: 30 is a nucleic acid sequence encoding an exemplary codon optimized soluble mouse Dectin-3 (sDectin-3) (SEQ ID NO: 31). The vector pET-45b+sequence of 9 codons is boxed with the start codon underlined. Sites for cloning into pET-45b+KpnI (GGTACC) (SEQ ID NO: 38) and PacI (TTAATTAA) (SEQ ID NO: 39), respectively, are underlined. Codons for Gly Ser (G,S) flexible linker residues are shown in bold. Reactive lys (K) codons (AAG) are shown in bold, with lysines in italic. Codon optimized sDectin-3 from the CLEC4D mouse Dectin-3 gene (GenBank Accesion No. NP_034949.3) is shown in plain text, with an Ala codon (GCT) and stop codons TAA and TTA underlined. Stop codons are shown in bold. The alternative gene name is MmsDectin3lyshis. The length of the nucleotide sequence is 604 base pairs, with 597 base pairs encoding a protein that is 199 amino acids in length. The nucleic acid encoding the exemplary codon-optimized mouse sDectin-3 was cloned into pET-45B+.

(SEQ ID NO: 30)

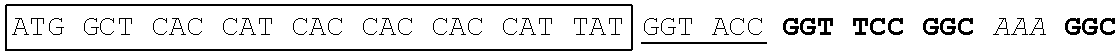

AAG GGC TCC GGC TCT GGT CAC
TATTTCCTGCGTTGGACCCGCGGTTCCGTGGTGAAACTGAGCGACTACCAT
ACGCGCGTGACTTGCATTCGTGAAGAGCCGCAGCCGGGCGCAACCGGCGGTACA
TGGACG
TGCTGCCCGGTTAGCTGGCGTGCGTTCCAGTCTAACTGTTATTTCCCACTGAATG
ACAAC
CAAACGTGGCATGAGAGCGAACGTAACTGCAGCGGCATGAGCAGTCACCTGGTT
ACCATT
AACACCGAGGCGGAGCAAAACTTTGTGACCCAATTGCTCGACAAGCGCTTCAGC
TACTTC
CTGGGTTTGGCCGATGAAAATGTTGAGGGTCAGTGGCAGTGGGTAGATAAGACC
CCGTTT
AATCCGCACACCGTCTTTTGGGAAAAGGGTGAGTCGAACGACTTCATGGAAGAA
GATTGT
GTTGTTCTGGTGCACGTGCACGAGAAGTGGGTTTGGAATGATTTCCCGTGTCATT
TTGAA
GTCAGACGTATCTGCAAATTACCGGGTATCACCTTTAACTGGAAACCGAGCAAA
GCT TAA TTA A

SEQ ID NO: 31 is an amino acid sequence encoded by SEQ ID NO: 30. This polypeptide comprises a mouse sDectin-3 protein. The N terminal amino acid and (His) 6 (HHHHHH) (SEQ ID NO: 19) affinity tag from pET-45B+is boxed. Gly Ser (GS) flexible linker residues and reactive lys (K) residues are shown in bold, with lysines in italic. Mouse sDectin-3 amino acid residues are shown in plain text (amino acids 23-199 of SEQ ID NO: 6), ending in a C-terminal Ala residue (A) in bold, the codon for which was used to put stop codons and PacI site in frame. The polypeptide is 199 amino acids in length with a MW of 23,023.72 g/mole and a theoretical pI or 6.52. In some examples, the soluble mouse Dectin-3 polypeptide comprises amino acids 23-198 of SEQ ID NO: 31.

(SEQ ID NO: 31)

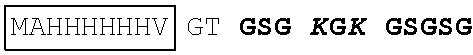

HYFLRWTRGSVVKLSDYHTRVTCIREEPQPGATGGTWTCCPVSWRAFQSNCYFPLN
DNQTWHESERNCSGMSSHLVTINTEAEQNFVTQLLDKRFSYFLGLADENVEGQWQ
WVDKTPFNPHTVFWEKGESNDFMEEDCVVLVHVHEKWVWNDFPCHFEVRRICKLP
GITFNWKPSK A

SEQ ID NO: 32 is a nucleic acid sequence encoding an exemplary codon optimized soluble human Dectin-1 (sDectin-1) (SEQ ID NO: 33). The human sDectin-1 DNA sequence is expressed from vector pET-45B+. The vector pET-45b+sequence and His tag of 9 codons is boxed with the start codon underlined. Cloning sites BamHI (GGATCC) (SEQ ID NO: 24) and PacI (TTAATTAA) (SEQ ID NO: 21), respectively, are underlined. Codons for enterokinase processing site in lower case font, Codons for Gly Ser (G,S) flexible linker residues and reactive lys (K) residues (AAA and AAG) are shown in bold with lysine codons in italic. The human sDectin-1 sequence (CLEC7A, GenBank Accession No. NM_197947) is shown in plain text, codon optimized for expression. An Ala codon GCT and stop codons TAA and TTA underlined, with stop codons in bold. An alternate name for this sequence is HssDectinllyshis. The nucleotide sequence encoding human sDectin-1 has a length of 649 base pairs, encoding a polypeptide that is 214 amino acids in length. The nucleic acid encoding the exemplary codon-optimized human sDectin-1 was cloned into pET-45B+.

(SEQ ID NO: 32)

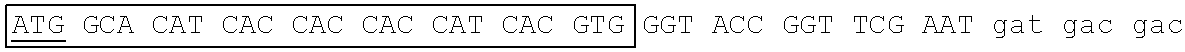

gac aag AGT CCG GAT CCC
GGA AGT GGA AAA GGC AAG GGT TCA GGG TCT GGC
ATCTGGCGTAGCAACAGCGGTAGCAAT
ACCCTGGAAAACGGTTACTTTTTGAGCCGCAACAAGGAGAACCATTCTCAGCCG
ACGCAA
AGCAGCTTGGAGGACTCGGTGACCCCAACGAAAGCTGTGAAGACCACGOGTGTC
CTGTCA
TCCCCGTGCCCCCGAATTOGATCATCTATGAGAAAAGCTOCTATCTCTTTAGCA
TGAGC
CTGAATAGCTGGGACGGCTCCAAACGTCAGTGTTGGCAGCTGGGCTCGAACTTG
CTGAAG
ATCGACTCCTCTAACGAATTAGGTTTCATTGTTAAGCAAGTTAGCAGCCAACCGG
ATAAT
TCCTTTTGGATTOGCCTGTCTCGTCCGCAGACCGAAGTTCCGTGGCTGTGGGAAG
ATGGT
TCCACTTTCAGCTCTAACCTGTTCCAGATTOGCACCACCOCAACCCAAGAGAATC
CTAGT
CCGAACTGCGTTTGGATTCACGTGAGCGTGATCTACGATCAGCTGTGTTCCGTCC
CGAGC
TACTCGATCTGCGAGAAAAAGTTCTCTATG GCT TAA TTA A

SEQ ID NO: 33 is an amino acid sequence encoded by SEQ ID NO: 32. This is a polypeptide comprising a human sDectin-1 protein. The N-terminal amino acid and (His) 6 (HHHHHH) (SEQ ID NO: 19) affinity tag from pET-45B+is boxed. The enterokinase processing site in lower case font. The Gly Ser (GS) flexible linker residues and reactive lys (K) residues are shown in bold, with lysines in italic. Human sDectin-1 amino acid residues are shown in plain text, ending in a C-terminal Ala residue (A) in bold, the codon for which was used to put stop codons and PacI site in frame. The polypeptide is 214 amino acids in length, with a MW of 23,703.20 g/mole and a theoretical pI of 6.22. In some examples, the soluble human Dectin-1 polypeptide fragment comprises amino acids 36-213 of SEQ ID NO: 33.

(SEQ ID NO: 33)

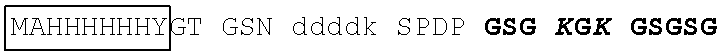

IWRSNSGSNTLENGYFLSRNKENHSQPTQSSLEDSVTPTKAVKTTGVLSSPCPPNWII
YEKSCYLFSMSLNSWDGSKRQCWQLGSNLLKIDSSNELGFIVKQVSSQPDNSFWIGL
SRPQTEVPWLWEDGSTFSSNLFQIRTTATQENPSPNCVWIHVSVIYDQLCSVPSYSICE
KKFSM A

SEQ ID NO: 34 is a nucleic acid sequence encoding an exemplary codon optimized soluble human Dectin-2 (sDectin-2) (SEQ ID NO: 35). The human sDectin-2 nucleotide sequence is expressed from vector pET-45B+. The length of the nucleotide sequence is about 616 base pairs with 580 base pairs encoding a protein of 203 amino acids in length. The vector pET-45b+sequence of 9 codons including the His tag is boxed with the start codon underlined. Cloning sites BamHI (GGATCC) (SEQ ID NO: 24) and PacI (TTAATTAA) (SEQ ID NO: 21), respectively, are underlined. Codons for enterokinase processing site in lower case font. Codons for Gly Ser (G,S) flexible linker residues are shown in bold, and reactive lys (K) residues (AAG) are shown in bold, with lysines in italic. Codon optimized sDectin-2 from the CLEC6A human Dectin 2 gene (cDNA GenBank Accession No. NM_001317999) is shown in plain text. An Ala codon (GCT) and stop codons, TAA and TTA, are underlined, with stop codons shown in bold. The alternative gene name is HssDectin2lyshis. The nucleic acid encoding the codon-optimized human sDectin-2 exemplary was cloned into pET-45B+.

(SEQ ID NO: 34)

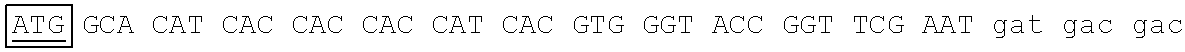

gac aag AGT CCG GAT CCC GGG TCT GGA AAA GGC AAG GGA AGT GGT TCA
GGC
ACCTACCACTTTACCTACGGCGAAACCGGTAAACGTCTGTCCGAGCTCCACTCAT
ATCACTCCTCTCTGACGTGCTTTAGCGAGGGTACTAAAGTGCCAGCGTGGGGTTG
TTGTCCGGCGAGCTGGAAGTCGTTCGGCAGCAGCTGCTATTTCATCAGCTCGGAG
GAAAAAGTTTGGAGCAAGAGCGAGCAAAACTGCGTGGAAATG
GGTGCACATTTGGTTGTCTTCAACACCGAAGCGGAGCAAAACTTTATCGTGCAGC
AGCTGAACGAAAGCTTCTCCTACTTCCTGGGTCTGTCCGACCCGCAGGGTAATAA
CAACTGGCAGTGGATTGATAAAACCCCGTATGAAAAGAACGTGCGCTTTTGGCA
TTTGGGCGAGCCGAATCATTCTGCCGAACAATGTGCGAGCATTGTTTTCTGGAAG
CCGACCGGCTGGGGTTGGAATGACGTTATTTGCGAGACGCGTCGTAACAGCATCT
GCGAGATGAATAAAATCTACCTG GCT TAA TTA A

SEQ ID NO: 35 is the amino acid sequence encoded by SEQ ID NO: 34. This polypeptide comprises a human sDectin-2 protein. The N-terminal amino acid and (His) 6 (HHHHHH) (SEQ ID NO: 19) affinity tag from pET-45B+ are boxed. The enterokinase processing site is in lower case font. Gly Ser (GS) flexible linker residues and reactive lys (K) residues are shown in bold, with lysines in italic. Human sDectin-2 amino acid residues are shown in plain text (GenBank Accession No. NP_001007034.1) (amino acids 36-203 of SEQ ID NO: 10), ending in a C-terminal Ala residue (A) in bold, the codon for which was used to put stop codons and PacI site in frame. The polypeptide is 203 amino acids in length, with a MW of 22,969 g/mole and a theoretical pI of 5.91. In some examples, the soluble human Dectin-2 polypeptide fragment comprises amino acids 35-202 of SEQ ID NO: 35.

(SEQ ID NO: 35)

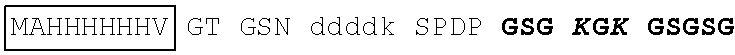

TYHFTYGETGKRLSELHSYHSSLTCFSEGTKVPAWGCCPASWKSFGSSCYFISSEEKV
WSKSEQNCVEMGAHLVVENTEAEQNFIVQQLNESFSYFLGLSDPQGNNNWQWIDKT
PYEKNVRFWHLGEPNHSAEQCASIVFWKPTGWGWNDVICETRRNSICEMNKIYL A

SEQ ID NO: 36 is a nucleic acid sequence encoding an exemplary codon optimized soluble human Dectin-3 (sDectin-3) (SEQ ID NO: 37). The human sDectin-3 DNA sequence is expressed from vector pET-45B+in E. coli. The vector pET-45b+sequence of 9 codons with hist tag is boxed, with the start codon underlined. Sites for cloning into pET-45b+BamHI (GGATCC) (SEQ ID NO: 24) and PacI (TTAATTAA) (SEQ ID NO: 21), respectively, are underlined. Codons for enterokinase processing site are in lower case font. Codons for Gly Ser (G,S) flexible linker residues are shown in bold and reactive lys (K) residues (AAG) are shown in bold, with lysines in italic. Codon optimized sDectin-3 from the CLEC4D human Dectin-3 gene, (GenBank Accession NM_080387) is shown in plain text. An Ala codon (GCT) and stop codons, TAA and TTA, are underlined, with stop codons in bold. The alternative gene name is HssDectin3lyshis. The nucleotide sequence has a length of 628 base pairs, encoding a polypeptide of 207 amino acids in length. The nucleic acid encoding the exemplary codon-optimized human sDectin-3 was cloned into pET-45B+.

(SEQ ID NO: 36)

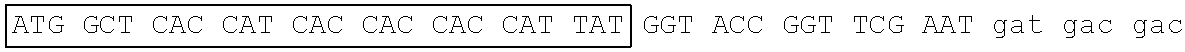

gac aag AGT CCG GAT CCC GGG TCT GGA AAA GGC AAG GGA AGT GGT TCA
GGC
CACAACTTCAGCCGTTGTAAGCGCGGTACGGGCGTGCATAAGTTGGAGCACCAC
GCCAAGCTCAAGTGCATCAAAGAAAAATCCGAGCTGAAATCTGCTGAGGGCAGC
ACCTGGAACTGCTGCCCGATTGATTGGCGTGCGTTTCAAAGCAATTGCTACTTCC
CGCTGACCGATAACAAAACCTGGGCGGAAAGCGAGCGCAACTGCAGCGGTATGG
GTGCACATCTGATGACCATTTCGACCGAAGCGGAGCAGAATTTCATCATCCAATT
TTTGGACCGCCGTCTGTCCTACTTCCTGGGTCTGCGTGATGAAAATGCAAAAGGC
CAATGGCGTTGGGTTGACCAGACCCCGTTTAACCCGCGTCGTGTTTTTTGGCATA
AGAACGAACCAGACAACAGCCAGGGTGAAAACTGCGTCGTGTTAGTTTATAACC
AGGATAAATGGGCGTGGAACGACGTGCCGTGTAATTTCGAGGCTTCTCGCATTTG
TAAGATCCCGGGTACGACTCTGAAT GCT TAA TTA A

SEQ ID NO: 37 is an amino acid sequence encoded by SEQ ID NO: 36. This polypeptide comprises the human Dectin-3 protein. The N-terminal amino acid and (His) 6 (HHHHHH) (SEQ ID NO: 19) affinity tag from pET-45B+is boxed. The enterokinase processing site is in lower case font. The Gly Ser (GS) flexible linker residues and reactive lys (K) residues are shown in bold, with lysines in italic. The human sDectin-3 amino acid residues (GenBank Accession No. NP_525126) are shown in plain text (amino acids 35-207 of SEQ ID NO: 12), ending in a C-terminal Ala residue (A) in bold, the codon for which was used to put stop codons and a PacI site in frame. The protein is 207 amino acids in length with a MW of 23,662 g/mole and a theoretical pI of 7.64. In some examples, the soluble human Dectin-3 polypeptide fragment comprises amino acids 35-206 of SEQ ID NO: 37.

(SEQ ID NO: 37)

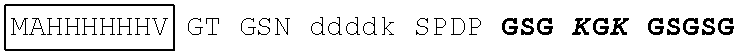

HNFSRCKRGTGVHKLEHHAKLKCIKEKSELKSAEGSTWNCCPIDWRAFQSNCYFPLT
DNKTWAESERNCSGMGAHLMTISTEAEQNFIIQFLDRRLSYFLGLRDENAKGQWRW
VDQTPFNPRRVFWHKNEPDNSQGENCVVLVYNQDKWAWNDVPCNFEASRICKIPG
TTLN A

Also provided is a liposome comprising a targeting molecule that binds a target antigen expressed by a bacterium or bacteria, wherein the targeting molecule is a C-Type Lectin polypeptide or a fragment thereof comprising a carbohydrate recognition domain (CRD), wherein the targeting molecule is incorporated into the outer surface of the liposome. In some embodiments, the targeting molecule is Dectin-1 polypeptide or a fragment thereof, comprising a CRD. In some embodiments, the Dectin-1 polypeptide comprises an amino acid sequence having at least 90% identity to SEQ ID NO: 20 or a fragment thereof. In some embodiments, the targeting molecule is Dectin-2 polypeptide or a fragment thereof, comprising a CRD. In some embodiments, the Dectin-2 polypeptide comprises an amino acid sequence having at least 90% identity t oSEQ ID NO: 21 or a fragment thereof. In some embodiments, the targeting molecule is a Dectin-3 polyeptide or a fragment thereof comprising a CRD. In some embodiments, the Dectin-3 polypeptide comprises an amino acid sequence having at least 90% identity to SEQ ID NO: 22. In some embodiments, the targeting molecule is a DC-SIGN polypeptide or a fragment thereof, comprising a CRD. In some embodiments, the DC-SIGN polypeptide comprises an amino acid sequence having at least 90% identity to SEQ ID NO: 40 or comprises an amino acid sequence having at least 90% identity to SEQ ID NO: 41. In some embodiments, the liposome comprises an antibacterial agent (e.g., a compound or drug) encapsulated in the liposome.

In some embodiments, the bacterial cell is a mycobacterial cell. In some embodiments, the mycobacterial cell is a Mycobacterium tuberculosis cell, a Mycobacterium avium cell or a Mycobacterium ulcerans cell. In some embodiments, the antibacterial agent is an antibiotic. In some embodiments, the antibacterial agent is selected from the group consisting of isoniazid. a rifamycin, rifapentine, rifabutin, pyrazinamide, and ethambutol. In some embodiments, the liposome or a plurality of liposomes comprising an antibacterial agent and a targeting molecule that binds a target antigen on a bacterial cell can target a population of any of the bacterial cells described herein, in vivo, ex vivo or in vitro. In some embodiments, any of the liposomes or pluralities of liposomes comprising an antibacterial agent can be used in any of the methods provided herein to treat a bacterial infection, for example a mycobacterial infection.

Also provided is a liposome comprising a targeting molecule that binds a target antigen expressed by a virus, wherein the targeting molecule is a C-Type Lectin polypeptide or a fragment thereof comprising a carbohydrate recognition domain (CRD), wherein the targeting molecule is incorporated into the outer surface of the liposome. In some embodiments, the targeting molecule is Dectin-1 polypeptide or a fragment thereof, comprising a CRD. In some embodiments, the Dectin-1 polypeptide comprises an amino acid sequence having at least 90% identity to SEQ ID NO: 20 or a fragment thereof. In some embodiments, the targeting molecule is Dectin-2 polypeptide or a fragment thereof, comprising a CRD. In some embodiments, the Dectin-2 polypeptide comprises an amino acid sequence having at least 90% identity to SEQ ID NO: 21 or a fragment thereof. In some embodiments, the targeting molecule is a Dectin-3 polyeptide or a fragment thereof comprising a CRD. In some embodiments, the Dectin-3 polypeptide comprises an amino acid sequence having at least 90% identity to SEQ ID NO: 22. In some embodiments, the targeting molecule is a DC-SIGN polypeptide or a fragment thereof, comprising a CRD. In some embodiments, the DC-SIGN polypeptide comprises an amino acid sequence having at least 90% identity to SEQ ID NO: 40 or an amino acid sequence having at least 90% identity to SEQ ID NO: 41. In some embodiments, the liposome further comprises an antiviral agent encapsulated in the liposome. In some embodiments, any of the liposomes or pluralities of liposomes comprising an antiviral agent can be used in any of the methods provided herein to treat or prevent a viral infection, for example, a coronavirus infection.

Also provided is a liposome comprising a targeting molecule that binds a target antigen expressed by a parasite, wherein the targeting molecule is a C-Type Lectin polypeptide or a fragment thereof comprising a carbohydrate recognition domain (CRD), wherein the targeting molecule is incorporated into the outer surface of the liposome, wherein the liposome is an antiparasitic agent. In some embodiments, the targeting molecule is Dectin-1 polypeptide or a fragment thereof, comprising a CRD. In some embodiments, the Dectin-1 polypeptide comprises an amino acid sequence having at least 90% identity to SEQ ID NO: 20 or a fragment thereof. In some embodiments, the targeting molecule is Dectin-2 polypeptide or a fragment thereof, comprising a CRD. In some embodiments, the Dectin-2 polypeptide comprises an amino acid sequence having at least 90% identity to SEQ ID NO: 21 or a fragment thereof. In some embodiments, the targeting molecule is a Dectin-3 polyeptide or a fragment thereof comprising a CRD. In some embodiments, the Dectin-3 polypeptide comprises an amino acid sequence having at least 90% identity to SEQ ID NO: 22. In some embodiments.

the targeting molecule is DC-SIGN polypeptide or a fragment thereof, comprising a CRD. In some embodiments, the DC-SIGN polypeptide comprises an amino acid sequence having at least 90% identity to SEQ ID NO: 40 or comprises an amino acid sequence having at least 90% identity to SEQ ID NO: 41. In some embodiments, the liposome further comprises an antiparasitic agent encapsulated in the liposome. In some embodiments, any of the liposomes or pluralities of liposomes comprising an antiparasitic agent can be used in any of the methods provided herein to treat a parasitic infection. In some embodiments, the parasitic infection is a protozoal infection or a helminthic infection. In some embodiments, the parasitic infection is a T. gondii infection.

The targeting molecules used to target nanoparticles, for example, liposomes, described herein can target nanoparticles of various compositions to an antigen on a pathogen or an antigen expressed on a pathogenic cell, for example, a fungal antigen, a viral antigen, a bacterial antigen or protozoan antigen. In other examples, the nanoparticles are targeted to an antigen on a parasite, for example, a helminth. Other examples of nanoparticles include, but are not limited to, iron oxide nanoparticles, polysaccharide gel nanoparticles and silica nanoparticles.

As used herein, the term liposome refers to an aqueous or aqueous-buffered compartment enclosed by at least one lipid bilayer. Optionally, liposomes can carry aqueous solutions, compounds, drugs or other substances in the compartment, i.e., internal cavity or space, enclosed by at least one lipid bilayer. Liposomes can vary in size, i.e., diameter. For example, a liposome can have a size of about 1000 nanometers (nm) or less. For example, a liposome can have a size of about 50 nm to about 1000 nm, about 50 nm to about 900 nm, about 50 nm to about 800 nm, about 50 nm to about 700 nm, about 50 nm to about 600 nm, about 50 nm to about 500 nm, about 50 nm to about 400 nm, about 50 nm to about 300 nm, about 50 nm to about 200 nm, or about 50 nm to about 100 nm. The liposomes described herein include liposomes comprising a compartment for encapsulation of an agent, for example, an antipathogenic agent (e.g., an antibacterial, an antiparasitic, an antiviral, an antiparasitic agent, or an antihelminthic agent, etc.): liposomes comprising a targeting molecule attached to or incorporated into the outside of the liposome and liposomes comprising an encapsulated antipathogenic agent. An encapsulated antipathogenic agent is an antipathogenic agent that is completely or partially located in the interior space of the liposome. For example, in any of the liposomes described herein, at least about 75%, 80%, 85%, 90%, 95% or 99% of the antipathogenic agent is incorporated into the interior space of the liposome or into the lipid bilayer of the liposome.

Any of the nanoparticles described herein, for example, liposomes, can contain about 1, 2, 3, 4, 5, 6, 7, 8, 9, 10, 11, 12, 13, 14, 15, 16, 17, 18, 19, 20 moles percent or greater of an antipathogenic agent relative to lipid. In other words, the nanoparticles can comprise a 1:100, 2:100, 3:100, 4:100, 5:100, 6:100, 7:100, 8:100, 9:100, 10:100, 11:100, 12:100, 13:100, 14:100, 15:100, 16:100, 17:100, 18:100, 19:100, 20:100 mole ratio of antipathogenic agent to liposomal lipid or greater. In some examples, the liposome contains about 1, 2, 3, 4, 5, 6, 7, 8, 9, 10, 11, 12, 13, 14, 15, 16, 17, 18, 19, 20 moles percent or greater of an antipathogenic agent, for example an antifungal, relative to lipid. In other words, the nanoparticles can comprise a 1:100. 2:100, 3:100, 4:100, 5:100, 6:100, 7:100, 8:100, 9:100, 10:100, 11:100, 12:100, 13:100, 14:100, 15:100, 16:100, 17:100, 18:100, 19:100, 20:100 mole ratio of antipathogenic agent to liposomal lipid or greater.

As used herein, mole ratio is the ratio between the amounts in moles of two components, for example, the ratio between the number of moles of the targeting molecule and the number of moles of lipid (targeting molecules: moles of lipid) or the number of moles of an antipathogenic agent and the number of moles of lipid (moles of antipathogenic agent: moles of lipid). And similarly, the nanoparticles can comprise a 0.002:100, 0.05:100, 0.1:100, 0.5:100. 1:100. 2:100, 3:100, 4:100, 5:100, 10:100, 15:100, 20:100, 25:100 mole ratio of targeting protein to liposomal lipid or greater.

Pluralities of two or more of any of the liposomes described herein are also provided. For example, a plurality of liposomes can comprise from about two to about 1×10¹⁴ (100 trillion) liposomes. For example, a plurality can have at least 100, 250, 500, 750, 1000, 5000, 10,000, 25,000, 50,000, 100,000, 500,000, 1 million or more liposomes. Liposomes can be made by any suitable method known to or later discovered by one of skill in the art. In general, liposomes can be prepared by a thin film hydration technique followed by a few freeze-thaw cycles. Liposomal suspensions can also be prepared according to methods known to those skilled in the art. Exemplary methods for the preparation of liposomes are described in Akbarzadeh et al. (“Liposome: classification, preparation and applications.” Nanoscale Res. Lett. 8 (1): 102 (2013)) which is hereby incorporated by reference in its entirety.

In general, a variety of lipid components can be used to make liposomes. These include neutral lipids that exist either in an uncharged or neutral zwitterionic form at physiological pH. Such lipids include, for example, diacylphosphatidylcholine, diacylphosphatidylethanolamine, ceramide, sphingomyelin, dihydrosphingomyelin, cephalin, and cerebrosides. Synthetic derivatives of any of the lipids described herein can also be used to make lipid nanoparticles. Lipid nanoparticles can also comprise a sterol, for example, cholesterol. Lipid nanoparticles can also comprise a cationic lipid which carries a net positive charge at about physiological pH. Such cationic lipids include, but are not limited to, N,N-dioleyl-N,N-dimethylammonium chloride (DODAC): N-(2,3-dioleyloxy) propyl-N,N-N-triethylammonium chloride (DOTMA); N,N-distearyl-N,N-dimethylammonium bromide (DDAB): N-(2,3-dioleoyloxy) propyl)-N,N,N-trimethylammonium chloride (DOTAP): 1,2-Dioleyloxy-3-trimethylaminopropane chloride salt (DOTAP.Cl): 3.beta.-(N--(N′.N′-dimethylaminoethane)-carbamoyl) cholesterol (“DC-Chol”). N-(1-(2,3-dioleyloxy) propyl)-N-2-(sperminecarboxamido)ethyl)-N,N-dimethyl-ammonium trifluoroacetate (“DOSPA”), dioctadecylamidoglycyl carboxyspermine (DOGS), 1,2-dileoyl-sn-3-phosphoethanolamine (DOPE), 1,2-dioleoyl-3-dimethylammonium propane (DODAP), N,N-dimethyl-2,3-dioleyloxy) propylamine (DODMA), and N-(1,2-dimyristyloxyprop-3-yl)-N,N-dimethyl-N-hydroxyethyl ammonium bromide (DMRIE).

Anionic lipids are also suitable for use in lipid nanoparticles described herein. These include, but are not limited to, phosphatidylglycerol, cardiolipin, diacylphosphatidylserine, diacylphosphatidic acid. N-dodecanoyl phosphatidylethanoloamine. N-succinyl phosphatidylethanolamine. N-glutaryl phosphatidylethanolamine, lysyl phosphatidylglycerol, and other anionic modifying groups joined to neutral lipids.

In some examples, the liposome comprises phosphatidylcholine, phosphatidylethanolamine, phosphatidylserine, phosphatidylinositol, phosphatidic acid, phosphatidylglycerol, palmitoyloleoyl phosphatidylcholine, lysophosphatidylcholine, lysophosphatidylethanolamine, dimyristoylphosphatidylcholine (DMPC), dipalmitoylphosphatidylcholine (DPPC), dioleoylphosphatidylcholine, distearoylphosphatidylcholine (DSPC), dilinoleoylphosphatidylcholine, a 1,2-distearoyl-sn-glycero-3-phosphoethanolamine (DSPE) conjugated polyethylene glycol (DSPE-PEG), a sphingomyelin, cholesterol, or any combination thereof. In some embodiments, PEG can be PEG-molecular weight (MW500) to PEG-MW20000. In addition to being components of the liposomes described herein, any of the lipids described herein can be conjugated to a targeting molecule or a fragment thereof that binds a target antigen. In some examples, pegylated versions of any of the lipids described herein can be conjugated to a targeting molecule or a fragment thereof that binds a target antigen on a pathogen, or an antigen expressed on a pathogenic cell. In some examples, the target antigen is on a pathogen. In some embodiments, the target antigen is on the cell wall of a pathogenic cell or is an antigen in an exopolysaccharide matrix associated with the pathogenic cell. In some examples, the target antigen is a fungal cell wall antigen or a fungal cell exopolysaccharide matrix antigen. In some examples the targeted antigen is a ligand of the binding molecule, or a ligand of the fragment of any of the binding molecules described herein.

As used throughout, a targeting molecule is a molecule that has a binding affinity for a target antigen, optionally a specific binding affinity, and can include, but is not limited to, an antibody, a polypeptide, a peptide, an aptamer, or a small molecule. As used throughout, an antigen expressed by a pathogen can be an antigen on a pathogen (for example, a virus, a fungus, or bacteria to name a few) or expressed by a pathogenic cell (for example, a fungal cell or a bacterial cell) at any stage of a pathogen's life cycle.

As used throughout, a pathogenic cell, for example a fungal cell or a bacterial cell, is a cell that typically causes infection. For example, and not to be limiting, an antigen can be associated with a pathogenic cell (for example, an antigen embedded in the fungal cell wall, an antigen attached to the fungal cell wall or a fungal cell surface antigen). An antigen associated with a pathogenic cell can also be directly or indirectly bound to the cell, for example, directly or indirectly bound to the cell wall. An antigen can also be an antigen of a pathogenic exopolysaccharide matrix, for example, a biofilm, produced by the pathogenic cell or associated with the pathogenic cell. In some examples, the exopolysaccharide matrix is adherent to or bound to the pathogenic cell or population of pathogenic cells. It is understood that an exopolysaccharide matrix associated with a pathogenic cell can be, but is not necessarily, produced by the pathogenic cell (for example, a fungal cell or bacterial cell) or the population of pathogenic cells it is associated with. As used throughout, an antigen can be, but is not limited to a protein, a lipid, or a carbohydrate.

As used throughout, polypeptide, protein and peptide are used interchangeably herein to refer to a polymer of amino acid residues. As used herein, the terms encompass amino acid chains of any length, including full-length proteins and fragments thereof, wherein the amino acid residues are linked by covalent peptide bonds.

As used throughout, the term nucleic acid refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991): Ohtsuka et al., J. Biol. Chem. 260:2605-2608 (1985); and Rossolini et al., Mol. Cell. Probes 8:91-98 (1994)).

In each case, where specific nucleic acid or polypeptide sequences are recited, embodiments comprising a sequence having at least 70% (e.g., 70%, 75%,80%, 85%, 90%, 95%, 99%) identity to the recited sequence are also provided. Identity or similarity with respect to a sequence is defined as the percentage of amino acid residues in the candidate sequence that are identical (i.e., same residue) with the starting amino acid residues, after aligning the sequences and introducing gaps, if necessary, to achieve the maximum percent sequence identity. For example, polypeptide and nucleic acid sequences having at least 70% (e.g., 70%, 75%, 80%, 85%, 90%, 95%, 99%) identity to SEQ ID NOs: 1-44 are provided herein. Polypeptides and nucleic acid sequences that do not include the histidine tag and/or linker sequences set forth in SEQ ID NOs: 1-44 are also provided. Polypeptides and nucleic acid sequences having at least 70% (e.g. 70%, 75%, 80%, 85%, 90%, 95%, 99%) identity to polypeptides and nucleic acid sequences that do not include the histidine tag and/or linker sequences set forth in SEQ ID NOs: 1-44 are also provided herein. Nucleic acids encoding the polypeptides described herein are also provided. Methods of alignment of sequences for comparison are well known in the art. Optimal alignment of sequences for comparison can be conducted, for example, by the local homology algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Any of the polypeptides disclosed herein can comprise one or more conservative amino acid substitutions. As a non-limiting example, the list below summarizes possible substitutions often likely to be carried out without resulting in a significant modification of the biological activity of the corresponding variant:
1) Alanine (A), Serine(S), Threonine (T), Valine (V), Glycine (G), and Proline (P):
2) Aspartic acid (D), Glutamic acid (E):
3) Asparagine (N), Glutamine (Q):
4) Arginine (R), Lysine (K), Histidine (H):
5) Isoleucine (I), Leucine (L), Methionine (M), Valine (V) and
6) Phenylalanine (F), Tyrosine (Y), Tryptophan (W).

See also, Creighton, Proteins, W. H. Freeman and Co. (1984).

In making such changes/substitutions, the hydropathic index of amino acids may also be considered. The importance of the hydropathic amino acid index in conferring interactive biologic function on a protein is generally understood in the art (Kyte and Doolittle: (1982) J Mol Biol. 157 (1): 105-32). It is accepted that the relative hydropathic character of the amino acid contributes to the secondary structure of the resultant protein, which in turn defines the interaction of the protein with other molecules, for example, enzymes, substrates, receptors, DNA, antibodies, antigens and the like.

As used throughout, the term antibody encompasses, but is not limited to, a nanobody, a whole immunoglobulin (i.e., an intact antibody) of any class, including polyclonal and monoclonal antibodies, as well as fragments of antibodies that retain the ability to bind their specific antigens. Also useful are conjugates of antibody fragments and antigen-binding proteins (single chain antibodies) as described, for example, in U.S. Pat. No. 4,704,692, the contents of which are hereby incorporated by reference in their entirety.

As used throughout, an aptamer is an oligonucleotide (single stranded DNA or single stranded RNA) or a peptide molecule that selectively bind to a target antigen. See, for example, Lakhin et al. “Aptamers: Problems, Solutions and Prospects,” Acta Naturae 5 (4): 34-43 (2013); and Reverdatto et al., “Peptide aptamers: development and applications,” Curr. Top Med. Chem. 15 (12): 1082-101 (2015)) hereby incorporated in their entireties by this reference.

As used herein, the terms specifically bind or selectively binds mean binding that is measurably different from a non-specific or non-selective interaction. Specific binding can be measured, for example, by determining binding of a molecule to a target antigen compared to binding of a control molecule. Specific binding can be determined by competition with a control molecule that is similar to the target antigen, such as an excess of non-labeled target antigen. In that case, specific binding is indicated if the binding of the labeled target to a probe is competitively inhibited by the excess unlabeled target antigen.

Optionally, from two targeting molecules to about 10,000 targeting molecules can be incorporated into the liposomes provided herein. For example, from about five to about one hundred, about five to about two hundred, about five to about three hundred, about five to about four hundred, about five to about five hundred, about five to about six hundred, about five to about seven hundred, about five to about eight hundred, about five to about nine hundred, about five to about one thousand, about five to about 1100, about five to about 1200, about five to about 1300, about five to about 1400, about five to about 1500, about five to about 1600, about five to about 1700, about five to about 1800, about five to about 1900, about five to about 2000, about five to about 2250, about five to about 2500, or about five to about 3000 targeting molecules, about five to about 3500, about five to about 4000 targeting molecules, about five to about 4500 targeting molecules, about five to about 5000 targeting molecules, about five to about 5500 targeting molecules, about 5 to about 6000 targeting molecules, about 5 to about 6500 targeting molecules, about 5 to about 7000 targeting molecules, about 5 to about 7500 targeting molecules, about 5 to about 8000 targeting molecules, about 5 to about 8500 targeting molecules, about 5 to about 9000 targeting molecules, about 5 to about 9500 targeting molecules or about 5 to about 10,000 can be incorporated into one or more liposomes described herein. In some examples, about two molecules to about 3,000 targeting molecules are incorporated into nanoparticles that are 100 nm in diameter. Those of skill in the art would not know how to calculate the number of targeting molecules that can be incorporated into a nanoparticle, for example between about two and 10,000 targeting molecules or greater depending on the size of the nanoparticle.

As used throughout, by incorporation of a targeting molecule into the outer surface of the liposome means that the targeting molecule is incorporated into the outer lipid bilayer of the liposome or attached to the liposome. Incorporation can occur by insertion or intercalation of the targeting molecule into the lipid bilayer. Attachment to a liposome can occur, for example, by affinity to a molecule incorporated into the outer lipid bilayer of the liposome. For example, the liposome can be coated with biotin (for example, DSPE-PEG-biotin inserted into the lipid bilayer) and the targeting molecule linked to streptavidin. Alternatively, the targeting molecule can be conjugated to the outer surface of the liposome. Targeting molecules can be conjugated to liposomes by a number of methods known in the art (e.g., Arruebo et al. “Antibody-Conjugated Nanoparticles for Biomedical Applications,” Journal of Nanomaterials vol. 2009, Article ID 439389 (2009)). Liposomes can also be conjugated to targeting molecules via a streptavidin/biotin bond, thiol/maleimide chemistry, azide/alkyne chemistry, tetrazine/cyclooctyne chemistry, and other click chemistries. These chemical handles are prepared either during phosphoramidite synthesis or post-synthesis. As used herein, the term click chemistry refers to biocompatible reactions intended primarily to join substrates of choice with specific biomolecules. Click chemistry reactions are not disturbed by water, generate minimal and non-toxic byproducts, and are characterized by a high thermodynamic driving force that drives it quickly and irreversibly to high yield of a single reaction product, with high reaction specificity.

### Compositions

Compositions comprising any of the liposomes described herein are also provided. Further provided are compositions comprising the polypeptides described herein. For example, a composition comprising a DC-SIGN polypeptide or fragment thereof is provided herein. Optionally, the polypeptide can comprise, consist essentially of, or consist of a CRD (SEQ ID NO: 1) and one or more neck regions of DC-SIGN selected from the group consisting of (NR1) SEQ ID NO: 2, (NR2) SEQ ID NO: 3, (NR3) SEQ ID NO: 4, (NR4) SEQ ID NO: 5, (NR5) SEQ ID NO: 6, (NR6) SEQ ID NO: 7, (NR7) SEQ ID NO: 8 and (NR8) SEQ ID NO: 9. In some compositions, the fragment comprises, consists essentially of, or consists of SEQ ID NO: 1, SEQ ID NO: 8 and SEQ ID NO: 9. In some compositions, the fragment comprises, consists essentially of, or consists of SEQ ID NO: 10. In some compositions, the fragment comprise, consists essentially of, or consists of SEQ ID NO: 1, SEQ ID NO: 2 and SEQ ID NO: 3. In some compositions, the fragment comprise, consists essentially of, or consists of SEQ ID NO: 12.

Optionally, the polypeptide is linked or conjugated to a fluorescent moiety, for example, and not to be limiting, rhodamine. The composition can comprise a buffer, for example, a renaturation buffer comprising from about 0.5M to about 1.5 M L-Arginine, as described in the Examples. For example, and not to be limiting, the compositions can comprise a buffer comprising between about 0.05 and 0.15 M NaH2PO4, between about 10 mM and 20 mM Triethanolamine, between about 0).5M and 1.5 M L-Arginine, between about 50 and 200 mM NaCl, between about 2.5 mM and 7.5 mM EDTA and between about 0.25 and 7.5 mM BME, at pH 7.2. Optionally, the compositions can comprise a buffer comprising about 0.1 M NaH2PO4, about 10 mM Triethanolamine, about 1 M L-Arginine, about 100 mM NaCl, about 5 mM EDTA and 5 mM BME, at pH 7.2. A kit comprising any of the compositions is also provided. Optionally, the kit comprises a denaturation buffer or reduction buffer, for example, a reduction buffer comprising beta-mercaptoethanol. Kits comprising any of the liposomes described herein are also provided.

### Pathogens

Any of the nanoparticles described herein can be used to target one or more pathogens, for example, bacteria, fungi, viruses, protists, and other single cell animal pathogens. The targeting molecule, for example, a C-Type Lectin polypeptide or fragment thereof (e.g., a DC-SIGN polypeptide, a Dectin-1 polypeptide, a Dectin-2 polypeptide, a Dectin-3 polypeptide or a fragment thereof described herein), can bind to an antigen on one or more types of pathogens or pathogenic cells. For example, a DC-SIGN polypeptide or fragment thereof, a Dectin-1 polypeptide or fragmeent thereof, a Dectin-2 polypeptide or a fragment thereof, or a Dectin-3 polypeptide or fragment thereof, can bind to one or more viruses: one or more fungal cells or populations of fungal cells: one or more animal pathogens (for example, a helminth): one or more protozoan cells or populations of protozoan cells: or one or more bacterial cells or populations of bacterial cells. Examples of viruses that can be targeted using any of the C-Type Lectin polypeptides or fragments thereof described herein, for example, a DC-SIGN polypeptide or fragment thereof, a Dectin-1 polypeptide or a fragment thereof, a Dectin-2 polypeptide or a fragment thereof, or a Dectin-3 polypeptide or fragment thereof, include, but are not limited to, SARS coronaviruses (for example, SARS-COV2), Influenza virus, HIV1 viruses, HIV2 viruses, Ebola Virus, Dengue virus, Herpes simplex virus 1, West-Nile virus, and Measles virus, to name few. In some embodiments, upon binding the virus, the liposome sequesters the virus, thus reducing replication of the virus and, in some cases, increasing clearance of the virus from a subject. In some embodiments, the DC-SIGN targeting molecule, or a fragment thereof comprising a CRD, binds to mannans on a spike protein of the SARS-CoV2 virus.

Examples of human fungal pathogens that can be targeted include, but are not limited to Aspergillus species such as A. fumigatus, Candida species such as C. albicans, C. glabrata. C. krusei, C. auris, Cryptococcus species such as C. gattii and C. neoformans, Talaromyces spp., Saccharomyces spp., Chrysosporium spp. In some of the methods and compositions described herein, the C-Type Lectin polypeptide or fragment thereof (for example, a DC-SIGN polypeptide, a Dectin-1 polypeptide or a Dectin polypeptide or fragment hereof) or a liposome comprising C-Type Lectin polypeptide or fragment thereof, does not target a fungal pathogen for detection (i.e., diagnostic), treatment or prevention purposes.

Examples of parasites that can be targeted by any of the liposomes described herein include helminths and protozoa. Helminths include, but are not limited to Schistosoma mansoni, Echinococcus granulosus, Trichinella spiralis, Anisakis spp. Diphyllobothrium spp. and Taenia spp. Examples of protozoa that can be targeted by any of the liposomes described herein include, but are not limited to Leishmania infantum, Toxoplasma gondii, Eimeria tenella, and Neospora caninum, Giardia intestinalis, Cyclospora cayetanensis and Plasmodium falciparum.

Examples of bacteria that can be targeted by any of the liposomes described herein include, but are not limited to Mycobacterium tuberculosis, Mycobacterium leprae, Mycobacterium bovis, Borrelia burgdorferi, Helicobacter pylori, Neisseria meningitidis.

### Antipathogenic Agents

The nanoparticles provided herein, for example, liposomes, can comprise one or more antipathogenic agents (e.g., an antiviral, an antibacterial, an antiparasitic, or an antifungal agent). In some examples, the concentration of the antipathogenic agent (e.g. effective dose) is reduced as compared to the concentration of the antipathogenic agent incorporated into or encapsulated in a liposome that does not comprise a targeting molecule or a fragment thereof incorporated into the outer surface of the liposome. In some cases, the concentration or amount of antipathogenic agent encapsulated in the liposome (e.g., effective dose) is reduced as compared to the concentration or amount of antipathogenic agent administered to the subject, when it is not incorporated into a liposome. In some liposomes, the reduction or decrease in the concentration of the antipathogenic drug is at least about 5%, 10%, 20%, 30%, 40%, 50%, 60%, 70%, 80%, 90%, 95% or any percentage in between these percentages. In some liposomes, the reduction or decrease in concentration of the antipathogenic drug is at least about a 2-fold, 3-fold, 4-fold, 5-fold, 6-fold, 7-fold, 8-fold, 9-fold, or 10-fold decrease. In some examples, the reduced concentration or amount of antipathogenic agent leads to a reduction in kidney cell toxicity and/or liver cell toxicity in vitro and/or in vivo.

### Antifungals

Antifungals that can be incorporated or encapsulated in the targeted liposomes described herein include but are not limited to a polyene or an azole antifungal. Examples of polyene antifungals include, but are not limited to, amphotericin B (AmB), candicidin, filipin, hamycin, natamycin, nystatin, hitachimycin and rimocidin. Examples of azole antifungals include, but are not limited to, imidazoles (for example, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, and Tioconazole), triazoles (for example, Albaconazole, Efinaconazole, Epoxiconazole, Fluconazole, Isavuconazole, Itraconazole, Posaconazole, Propiconazole, Ravuconazole, Terconazole, Voriconazole thiazoles (for example, abafungin) and echinocandins (for example, caspofungin, micafungin and anidulafungin).

Amphotericin B (AmB) is a commonly used agent for many kinds of fungal infections, including aspergillosis. The side effects of Amphotericin B include neurotoxicity and/or nephrotoxicity and/or hepatoxicity and often result in death of the patient. AmB is hydrophobic and is intercalated into the lipid bilayer of liposomes. Commercial untargeted spherical AmB loaded liposomes, AmB-LLs, are often referred to as AmBisomes. AmB-LLs penetrate more efficiently to various organs, penetrate the cell wall and show reduced toxicity at slightly higher, more effective doses of AmB than the second most commonly used AmB product, deoxycholate detergent solubilized AmB. However, AmB-LLs still produce AmB human toxicity, such as renal toxicity in 50% of patients. When infected mice are treated with AmB-LLs, large fungal cell populations often remain. This large residual population is likely a reason that detergent solubilized AmB and AmB-LL treated human patients have high rates of recurrence and subsequent mortality after treatment. The targeted liposomes provided herein are designed to effectively target fungal cells and/or reduce toxicity of the antifungal agent, for example, AmB.

In some examples, the concentration of the antifungal drug is reduced as compared to the concentration of the antifungal drug incorporated into or encapsulated in a liposome that does not comprise a targeting molecule or a fragment thereof incorporated into the outer surface of the liposome, wherein the targeting molecule binds an antigen on a fungal cell. By forming protein-coated liposomes, i.e., coating the liposomes with a polypeptide, for example, a DC-SIGN polypeptide or fragment thereof described herein, lower concentrations of the antifungal agent can be used to treat or prevent a fungal infection, thus decreasing the toxicity associated with administration of the antifungal drug to the subject. Lower toxicity allows extended use of the targeted antifungal agent over longer periods, which would reduce the fungal load beyond the poor reduction that is currently achieved. Lower toxicity could allow the targeted liposomes described herein to be used prophylactically, for example, as a nasal spray, to prevent lung infections before they are established.

In some liposomes, the reduction or decrease in the concentration of the antifungal drug is at least about 5%, 10%, 20%, 30%, 40%, 50%, 60%, 70%, 80%, 90%, 95% or any percentage in between these percentages. In some liposomes, the reduction or decrease in concentration of the antifungal drug is at least about a 2-fold, 3-fold, 4-fold, 5-fold, 6-fold, 7-fold, 8-fold, 9)-fold, or 10-fold decrease. In some examples, the reduction in toxicity is a reduction in kidney cell toxicity and/or liver cell toxicity in vitro and/or in vivo. In another example, the reduction in the concentration of AmB in the liposome is reduced from about 11 moles percent relative to liposomal lipid to about 1 to 10 moles percent relative to liposomal lipid. In some targeted liposomes, the concentration of the antifungal agent can range from about 1 to about 20 moles percent antifungal agent relative to percent liposomal lipid. For example, the concentration of the concentration of the antifungal agent can be about 1, 2, 3, 4, 5, 6, 7, 8, 9, 10, 11, 12, 13, 14, 15, 16, 17, 18, 19 or 20 moles percent antifungal agent relative to percent liposomal lipid.

### Antiviral Agents

Antiviral agents include, but are not limited to, Abacavir, Acyclovir (Aciclovir), Adefovir, Amantadine, Ampligen, Amprenavir (Agenerase), Umifenovir (Arbidol), Atazanavir, Atripla, Baloxavir marboxil (Xofluza), Biktarvy, Boceprevir, Bulevirtide, Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Edoxudine, Efavirenz, Elvitegravir, Emtricitabine, Enfuvirtide, Entecavir, Etravirine (Intelence), Famciclovir, Fomivirsen, Fosamprenavir, Foscarnet, Ganciclovir (Cytovene), Ibacitabine, Idoxuridine, Imiquimod, Imunovir, Indinavir, Lamivudine, Letermovir (Prevymis), Lopinavir, Loviride, Maraviroc, Methisazone, Moroxydine, Nelfinavir, Nevirapine, Nexavir formerly (Kutapressin), Nitazoxanide, Norvir, Oseltamivir (Tamiflu), Penciclovir, Peramivir, Penciclovir, Peramivir

Pleconaril, Podophyllotoxin, Raltegravir, Remdesivir, Ribavirin, Rilpivirine (Edurant), Rilpivirine, Rimantadine, Ritonavir, Saquinavir, Simeprevir (Olysio), Sofosbuvir, Stavudine, Taribavirin (Viramidine), Telaprevir, Telbivudine (Tyzeka), Tenofovir alafenamide, Tenofovir disoproxil, Tipranavir, Trifluridine, Trizivir, Tromantadine, Truvada, Umifenovir, Valaciclovir (Valtrex), Valganciclovir (Valcyte), Vicriviroc, Vidarabine, Zalcitabine, Zanamivir (Relenza), and Zidovudine.

### Antibacterial Agents

Antibacterial agents include, but are not limited to, Doxycycline, Minocycline, Aminoglycosides, Ampicillin, Amoxicillin/clavulanic acid (Augmentin), Azithromycin, Carbapenems (e.g. imipenem), Clarithromycin, Metronidazole, and Amoxicilli, Piperacillin/tazobactamErythromycin, Cycloserine, Capreomycin, Framycetin, Oxytetracycline, Rifabutin, Bacitracin, Benzylpenicillin, Streptomycin, Gramicidin D, Penicillin (e.g. G), Naficillin/oxacillin, Ceftriaxone, Cefepime, Cefotaxime, Ertapenem, Imipenem, Ciprofloxacin, Levofloxacin, Gentamycin, Vancomycin, Bactrim, Kanamycin, Capreomycin, Amikacin, Viomycin, isoniazid, a rifamycin, rifapentine, rifabutin, pyrazinamide, and ethambutol.

### Antihelminthic Agents

Antihelminthic agents include, but are not limited to, Ivermectin, pyrantel, pyrantel, albendazole, mebendazole, praziquantel, mebendazole, pyrantel, pyrantel, thiabendazole, miltefosine, triclabendazole, and pyrantel.

### Antiprotozoan Agents

Antiprotozoan agents include, but are not limited to, Mefloquine, Chloroquine, Proguanil, Primaquien, atovaquone, Doxycycline, Metronidazole, Tinidazole, Nifuratel, Praziquantel, Miltefosine, Oxaminiquine, Pyrimethamine and Sulfadiazine, Leucovorin, Amprolium, and Salinomycin.

In some examples, the targeted liposome has decreased affinity for and/or is less toxic to an animal cell, for example, a human cell, as compared to a liposome that does not comprise a targeting molecule that binds an antigen on a pathogen or pathogenic cell, for example, a targeting molecule incorporated into the outer surface of the liposome. In some examples, the targeted liposomes have a higher affinity for pathogenic cells, for example, fungal cells, in the lungs, kidney or liver cells of a subject as compared to the affinity of the targeted liposomes for lung, kidney or liver cells of the subject. Therefore, the liposomes provided herein can be used to deliver an antipathogenic agent to a subject while minimizing the effects of the antipathogenic agent on non-pathogenic cells, for example, human lung, kidney or liver cells, thereby reducing the toxicity of the antipathogenic agent. Any of the liposomes comprising an antipathogenic agent described herein can be used to reduce or decrease an infection in vitro, ex vivo or in vivo.

### Methods for Making Targeted Liposomes

Provided herein is a method of making a plurality of liposomes comprising an antipathogenic agent (for example, an antifungal agent, an antiviral agent, an antibacterial agent, an antihelminthic agent or an antiprotozoan agent) and a targeting molecule that binds a target antigen, wherein the targeting molecule is incorporated into the outer surface of each liposome and the antipathogenic agent is encapsulated in each liposome, the method comprising the steps of (a) dissolving the antipathogenic agent in solvent for about 10 minutes to about 30 minutes, at about 60° C. (b) encapsulating the antipathogenic agent into each liposome by mixing a plurality of liposomes in suspension with the antipathogenic/solvent solution of step (a), for about 3 to about 5 hours, at about 60° C. or at about 37° C. for about 24-120 hours; and (c) incorporating the targeting molecule into the outer surface of each liposome by contacting the liposomes comprising the encapsulated antipathogenic agent with the targeting molecule conjugated to a lipid, for about 45 minutes to about 90 minutes, at 60° C.

In the methods of making liposomes provided herein, the antipathogenic agent can be dissolved in any suitable solvent. Depending on the antipathogenic agent and its properties, one of skill in the art would know how to select an effective solvent for dissolution. In some examples, the antipathogenic agent is an antifungal agent, for example, amphotericin B, which can be dissolved in aqueous DMSO or formamide. Other hydrophobic or amphiphobic solvents can also be used. Optionally, the antipathogenic agent can be dissolved for about 10 to about 120 minutes. For example, the antipathogenic agent can be dissolved for about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or 120 minutes.

Optionally, the antipathogenic agent can be dissolved at a temperature of about 55° C. to about 65° C., for example, at about 55, 56, 57, 58, 59, 60, 61, 62, 63, 64 or 65 degrees Celsius. Optionally, the antipathogenic agent is encapsulated into each liposome by mixing a plurality of liposomes in suspension with the antifungal/solvent solution (dissolved antifungal agent) for about 3 to about 5 hours, at about 55° C. to about 65° C., for example, at about 55, 56, 57, 58, 59, 60, 61, 62, 63, 64 or 65 degrees Celsius. In another example, the antipathogenic agent is encapsulated into each liposome by mixing a plurality of liposomes in suspension with the antipathogenic agent/solvent solution (dissolved antifungal agent) at about 35° C. to about 45° C., for example, at about 35, 36, 37, 38, 39, 40, 41, 42, 43, 44, or 45 degrees Celsius, for about 24-120 hours. In another example, the antipathogenic agent is encapsulated into each liposome by mixing a plurality of liposomes in suspension with the antipathogenic/solvent solution (dissolved antipathogenic agent) at about 35° C. to about 45° C., for example, at about 35, 36, 37, 38, 39, 40, 41, 42, 43, 44, or 45 degrees Celsius, for about 72-100 hours.

Optionally, in any of the methods for making liposomes described herein, the C-type lectin receptor or fragment thereof, for example, DC-SIGN, is maintained in a renaturation buffer comprising arginine and denatured prior to incorporation into the liposome. Optionally, any of the methods for making liposomes described herein can further comprise storing the liposomes comprising an antipathogenic agent and a targeting molecule in a renaturation buffer comprising arginine. Optionally, the renaturation buffer can comprise about 0.5 to about 1.5M arginine. Optionally, the renaturation buffer can comprise about 0.1 M NaH2PO4, about 10 mM Triethanolamine, about 1 M L-Arginine, about 100 mM NaCl, about 5 mM EDTA and 5 mM BME, at pH 7.2.

In some methods, the targeting molecule is conjugated to a lipid. The lipid conjugated to the targeting molecule can be a pegylated or a non-pegylated lipid. Examples of lipids that can be conjugated to the targeting molecule and examples of antifungal agents that can be incorporated into targeted liposomes are set forth above. In some examples, the targeting molecule incorporated into the outer surface of each liposome is a C-type lectin receptor, for example. DC-SIGN or a fragment thereof.

Pluralities of targeted liposomes made by the methods described can produce pluralities comprising any number of liposomes, for example, from about two to about 100,000,000 liposomes. For example, pluralities comprising about 100, 500, 1000, 5,000, 10,000, 15,000, 25,000, 50,000, 100,000, 200,000, 300,000, 500,000, 600,000, 700,000, 800,000, 900,000, 1,000,000 or more liposomes are provided. It is understood that during the process of making targeted liposomes there may be some liposomes that do not encapsulate the antifungal agent or liposomes that do not have a targeting molecule incorporated into the outer surface of the liposomes. Therefore, provided herein are pluralities of targeted liposomes wherein at least 70%, 80%, 90%, 95%, 99% 99.5%, or 99.9% of the liposomes have an encapsulated antifungal agent and a targeting molecule that binds an antigen on a fungal cell, wherein the targeting molecule is incorporated into the outer surface of the liposomes.

### Detection of Infection

Also provided herein are liposomes comprising a targeting molecule (e.g., a DC-SIGN polypeptide or fragment thereof, a Dectin-1 polypeptide or fragment thereof, a Dectin-2 polypeptide or fragment thereof, or a Dectin-3 polypeptide or fragment thereof) that binds a target fungal cell antigen and a signal-generating molecule, wherein the targeting molecule is incorporated into the outer surface of the liposome, and the signal-generating molecule generates a detectable signal when the targeting molecule binds the target antigen on a pathogen, a pathogenic cell or material released from a pathogenic cell. In some examples, the signal-generating molecule is linked to or attached to the targeting molecule. In other examples, the signal-generating molecule is incorporated into or attached to the outer surface of the liposome. These liposomes can be used to detect the presence of a pathogen or a pathogenic infection, in vivo, ex vivo or in vitro. A pathogenic cell or one or more pathogenic cells, i.e., a population of pathogenic cells, can be detected. The detectable signal can be directly or indirectly detected. For example, the signal-generating molecule can be a fluorescent dye, label or probe that is directly detected (for example, rhodamine, fluorescein, green fluorescent protein, acridine orange, etc.).

In some examples, the targeting molecule is linked to a molecule that can be directly detected in vivo using imaging techniques, including, but not limited to magnetic resonance imaging, radiography, position emission tomography (PET), computed tomography (CT) scan, to name a few. Examples of molecules that can be used for in vivo imaging include, but are not limited to, a metalloprotein, ferritin, transferrin, aquaporin, and a chemical exchange saturation transfer (CEST) report, to name a few. See, for example, Silindir et al. “Liposomes and their applications in molecular imaging.” J. Drug Target 20 (5): 401-415 (2012); and Mukherjee et al. “Biomolecular MRI Reporters: evolution of new mechanisms,” Prog. Nucl. Magn Reson. Spectrosc. 102-103:32-42 (2017)). In another example, the targeting molecule is linked to a primary antibody or a fragment thereof (for example, an Fc fragment of an antibody) that can be indirectly detected using a secondary antibody.

In some examples, the liposome itself generates a signal when bound to a target antigen on a pathogen or a pathogenic cell. For example, flow cytometry can be used to detect increased signal intensity, light scattering, and sizes of multiple fluorescent liposomes bound to virus particles or fragments of polysaccharide released from a pathogen, present in the subject (for example, the eye, ear, throat, vagina, nasal passage, skin or nail of a subject, to name a few) or a sample from the subject (for example, urine, blood, serum, tears, speutum, lung lavage, tissue scraping or homogenate) as contrasted with normal properties of fluorescent liposomes not bound to pathogenic material. For example, duplicate samples of potentially infected material are mixed with targeted fluorescent liposomes and untargeted fluorescent liposomes. The cytometer would be adjusted such that forward light scattering and side light scattering would detect particles of the size of the pathogen or larger. Because of low concentrations of pathogen particles, the pathogen is unlikely to be detected above background in the sample mixed with untargeted fluorescent liposomes. But in the sample mixed with targeted fluorescent liposomes, there would be a signal, for example, a red fluorescent signal, at this position, because the pathogen is coated with fluorescent liposomes. If, for example, the pathogen is a virus, its small size as indicated by light scattering in the targeted liposome sample would likely be larger, due to a number of fluorescent liposomes bound.

Also provided herein is a liposome comprising a targeting molecule, for example, a C, type lectin polypeptide or fragment thereof described herein (e.g., a DC-SIGN polypeptide or fragment thereof, a Dectin-1 polypeptide or fragment thereof, a Dectin-2 polypeptide or fragment thereof, or a Dectin-3 polypeptide or fragment thereof), that binds a target antigen on a pathogenic cell, wherein the targeting molecule is incorporated into the outer surface of the liposome, and wherein the targeting molecule is linked or fused to a C-terminal or an N-terminal fragment of a fluorescent protein. Therefore, the liposome comprises a fusion protein comprising a targeting molecule that binds to a pathogenic cell antigen and the C-terminal or the N-terminal of a fluorescent protein. Pluralities of these liposomes are also provided. In some examples, the plurality includes a first subset of liposomes comprising a targeting molecule linked to an N-terminal fragment of a fluorescent protein and a second subset of liposomes comprising a targeting molecule linked to a C-terminal fragment of a fluorescent protein. In this example, the multimers of the protein brought about by binding the cognate ligand or antigen would create a fluorescent signal in what is known as bimolecular fluorescence complementation.

Provided herein is a method for detecting an infection in a subject or a sample from the subject comprising (a) contacting the subject or a sample from the subject with a plurality of liposomes, wherein each liposome in the plurality comprises: a) a targeting molecule that binds a target antigen on a pathogen or a pathogenic cell, and: b) a signal-generating molecule, wherein the targeting molecule is incorporated into the outer surface of the liposome, and the signal-generating molecule generates a detectable signal when the targeting molecule binds the target antigen; and b) detecting a signal, wherein a signal indicates the presence of an infection. This method can be used to detect an infection in vivo, ex vivo or in vitro. In some examples, the target antigen is an antigen on a viral particle. In other examples, the target antigen is fungal cell antigen on a cell. In other examples, the fungal cell antigen is a soluble fungal cell antigen, for example, beta-glucans or mannans in a biological sample. In some examples, the targeting molecule is linked to the signal-generating molecule. In other examples, the signal-generating molecule is incorporated into or attached to the outer surface of the liposome. In some examples, the targeting molecule is linked to a signal generating enzyme, for example, HRP, luciferase, beta-glucuronidase, and beta-galactosidase. In other examples, the targeting molecule is linked to a fluorescent protein, for example, rhodamine, GFP, YFP, RFP, etc. Fragments of fluorescent proteins, for example, the N-terminal or the C-terminal of any fluorescent protein can be linked to the targeting molecule. In other examples, the targeting molecule is linked to antibody, or a fragment thereof.

In some examples, the plurality of liposomes is immobilized on a solid support. Non-limiting examples of solid support materials include glass, modified or functionalized glass, plastics including acrylics, polystyrene and copolymers of styrene and other materials, polypropylene, polyethylene, poly butylene, polyurethanes, or TeflonJ, nylon, nitrocellulose, polysaccharides, resins, silica or silica-based materials including silicon and modified silicon, carbon, metals, inorganic glasses and plastics. The size and shape of the solid support can vary. A solid support can be planar, a solid support can be a well, or alternatively, a solid support can be a bead or a slide. In some embodiments, a solid support is a well of a multiwell plate. In other examples, the solid support can be a magnetic bead, an agarose-based resin, or an agarose bead. In other examples, the solid support comprises non-agarose chromatography media, monoliths, or nanoparticles. For example, the chromatography media can be, e.g., methacrylate, cellulose, or glass. In other examples, the nanoparticles are gold nanoparticles or magnetic nanoparticles.

As used throughout, by subject is meant an individual. The subject can be an adult subject or a pediatric subject. Pediatric subjects include subjects ranging in age from birth to eighteen years of age. Thus, pediatric subjects of less than about 10 years of age, five years of age, two years of age, one year of age, six months of age, three months of age, one month of age, one week of age or one day of age are also included as subjects. Preferably, the subject is an animal, for example, a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

As used herein, a biological sample is a sample derived from a subject and includes, but is not limited to, any cell, tissue or biological fluid. The sample can be, but is not limited to, blood, plasma, serum, sputum, urine, saliva, bronchoalveolar lavage fluids, biopsy (e.g., tissue or cells isolated from organ tissue, for example, from lung, liver, kidney, skin etc.), vaginal secretion, nasal secretion, skin, gastric secretion, or bone marrow specimens.

### Methods for Reducing Infection

Provided herein are methods for using any of the liposomes or plurality of liposomes described herein to inhibit or reduce infection in vitro, ex vivo or in vivo. As used throughout, inhibition or reduction does not have to be complete and can be a reduction of at least 5%, 10%, 15%, 20%, 25%, 30%, 35%, 40%, 45%, 50%, 55%, 6-%, 65%, 70%, 75%, 80%, 85%, 90%, 95%, 100% or any percentage in between these percentages, in vivo, ex vivo or in vitro. Any phase of a pathogen's life cycle can be inhibited including, but not limited to, attachment to cellular receptors, infection, cellular entry internalization, and replication. When the pathogen is a virus, these phases can also include disassembly of the virus, viral replication, genomic integration of viral sequences, transcription of viral RNA, translation of viral mRNA, assembly of viral particles, budding, cell lysis and egress of virus from the cells.

Also provided are methods for treating or preventing an infection in a subject. The methods comprise administering to the subject having an infection or at risk of developing an infection an effective amount of a plurality of any of the liposomes described herein. In some examples, the at least 90%, 95%, 99% or more of the liposomes in the plurality comprise a targeting molecule that binds a target antigen on a pathogen or pathogenic cell. In some examples, each liposome in the plurality comprises a targeting molecule that binds a target antigen on a pathogen or pathogenic cell, wherein the targeting molecule is incorporated into the outer surface of the liposome and the liposome does not comprise an antipathogenic agent encapsulated in the liposome (e.g., a drug encapsulated in the liposome). In some examples, each liposome in the plurality comprises an antipathogenic agent and a targeting molecule that binds a target antigen, wherein the targeting molecule is incorporated into the outer surface of the liposome and the antipathogenic agent is encapsulated in the liposome. Any of the liposomes or pluralities of liposomes provided herein can be in a pharmaceutical composition.

The methods can be used to treat or prevent an infection in any animal. Examples of viral, fungal, bacterial, parasitice (e.g., protozoan and helminthic) infections are described above. In some methods for treating or preventing an infection, the infection is not a fungal infection.

Throughout, treat, treating, and treatment refer to a method of reducing or delaying one or more effects or symptoms of an infection. The subject can be diagnosed with an infection. Treatment can also refer to a method of reducing the underlying pathology rather than just the symptoms. The effect of the administration to the subject can have the effect of, but is not limited to, reducing one or more symptoms of the disease, a reduction in the severity of the disease, the complete ablation of the disease, or a delay in the onset or worsening of one or more symptoms. For example, a disclosed method is considered to be a treatment if there is about a 10% reduction in one or more symptoms of the disease in a subject when compared to the subject prior to treatment or when compared to a control subject or control value. Thus, the reduction can be about a 10, 20, 30, 40, 50, 60, 70, 80, 90, 100%, or any amount of reduction in between.

As used herein, by prevent, preventing, or prevention is meant a method of precluding, delaying, averting, obviating, forestalling, stopping, or hindering the onset, incidence, severity, or recurrence of a disease or disorder. For example, the disclosed method is considered to be a prevention if there is a reduction or delay in onset, incidence, severity, or recurrence of an infection in a subject susceptible to an infection or recurrence of an infection compared to control subjects susceptible to a fungal infection or recurrence of an infection that did not receive treatment. The reduction or delay in onset, incidence, severity, or recurrence of an infection can be about a 10, 20, 30, 40, 50, 60, 70, 80, 90, 100%, or any amount of reduction in between.

In some methods, the subject is immunocompromised. For example, the subject can be a subject that has undergone a stem cell, organ, tissue or bone marrow transplant, a subject that has cancer, a subject receiving cancer therapy (for example, chemotherapy, immunotherapy or radiotherapy), a subject taking corticosteroids, a subject infected with HIV or having acquired immunodeficiency syndrome, a subject that has hepatitis, a subject with a B-cell defect or a subject with a T-cell defect, to name a few:

In some methods, the subject has one or more disorders that affect lung function in the subject, for example, pulmonary fibrosis, pneumonia, asthma, chronic obstructive pulmonary disease (COPD), cystic fibrosis, tuberculosis, emphysema or sarcoidosis.

The methods provided herein optionally include selecting a subject with an infection or at risk of developing an infection. One of skill in the art knows how to diagnose a subject with an infection. For example, a medical examination can be performed. One or more of the following tests can be also used: microscopic examination of clinical samples, histopathology. culture, and serology. Molecular diagnostics and antigen detection in clinical samples can also be used (See, for example, Kozel and Wickes “Fungal Diagnostics.” Cold Spring Harb. Perspect. Med. 4 (4): a019299 (2014)).

The methods provided herein optionally further include administering an effective amount of a second therapeutic agent or therapy to the subject. The second therapeutic agent or therapy can be administered to the subject prior to, simultaneously with, or subsequent to administration of the plurality of liposomes. In some methods, the second therapeutic therapy is surgery. In some methods, the second therapeutic agent is a second antifungal agent. In some methods, the second therapeutic agent is a second antiviral agent. In some methods, the second therapeutic agent is a second antihelminthic agent. In some methods, the second therapeutic agent is a second antiprotozoan agent. The antifungal agent can be any of the polyene antifungals, azole antifungals, imidazoles, triazoles or echinocandins described above. In some methods, a first infection in the subject, for example, a viral infection is treated with liposomes comprising an antiviral agent and a second infection in the subject for example, a bacterial infection, is treated with an antibacterial agent. It is understood that some agents can be used to treat or prevent more than one type of infectious disease. For example, in some embodiments, an antibacterial agent can also be used as an antiparasitic agent.

Antiviral agents include but are not limited to Abacavir, Acyclovir (Aciclovir), Adefovir, Amantadine, Ampligen, Amprenavir (Agenerase), Umifenovir (Arbidol) Atazanavir, Atripla, Baloxavir marboxil (Xofluza), Biktarvy, Boceprevir, Bulevirtide, Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Edoxudine, Efavirenz, Elvitegravir, Emtricitabine, Enfuvirtide, Entecavir, Etravirine (Intelence), Famciclovir, Fomivirsen, Fosamprenavir, Foscarnet, Ganciclovir (Cytovene), Ibacitabine, Idoxuridine, Imiquimod, Imunovir, Indinavir, Lamivudine, Letermovir (Prevymis), Lopinavir, Loviride, Maraviroc, Methisazone, Moroxydine, Nelfinavir, Nevirapine, Nexavir formerly (Kutapressin), Nitazoxanide, Norvir, Oseltamivir (Tamiflu), Penciclovir, Peramivir, Penciclovir, Peramivir, Pleconaril, Podophyllotoxin, Raltegravir, Remdesivir, Ribavirin, Rilpivirine (Edurant), Rilpivirine, Rimantadine, Ritonavir, Saquinavir, Simeprevir (Olysio), Sofosbuvir, Stavudine, Taribavirin (Viramidine), Telaprevir, Telbivudine (Tyzeka), Tenofovir alafenamide, Tenofovir disoproxil, Tipranavir, Trifluridine, Trizivir, Tromantadine. Truvada, Umifenovir, Valaciclovir (Valtrex), Valganciclovir (Valcyte), Vicriviroc, Vidarabine, Zalcitabine, Zanamivir (Relenza), and Zidovudine.

Antibacterial agents include, but are not limited to, Doxycycline, Minocycline, Aminoglycosides, Ampicillin, Amoxicillin/clavulanic acid (Augmentin), Azithromycin, Carbapenems (e.g. imipenem), Clarithromycin, Metronidazole, and Amoxicilli, Piperacillin/tazobactamErythromycin, Cycloserine, Capreomycin, Framycetin, Oxytetracycline, Rifabutin, Bacitracin, Benzylpenicillin, Streptomycin, Gramicidin D, Penicillin (e.g. G), Naficillin/oxacillin, Ceftriaxone, Cefepime, Cefotaxime, Ertapenem, Imipenem, Ciprofloxacin, Levofloxacin, Gentamycin, Vancomycin, Bactrim, Kanamycin, Capreomycin, Amikacin, Viomycin, isoniazid, a rifamycin, rifapentine, rifabutin, pyrazinamide, and ethambutol.

Antiparasitic agents include antihelmintic agents as well as antiprotozoan agents. Antihelminthic agents include, but are not limited to, ivermectin, pyrantel, pyrantel, albendazole, mebendazole, praziquantel, mebendazole, pyrantel, pyrantel, thiabendazole, miltefosine, triclabendazole, and pyrantel. Antiprotozoan agents include, but are not limited to, Mefloquine, Chloroquine, Proguanil, atovaquone, Doxycycline, Metronidazole, Tinidazole, Nifuratel, Praziquantel, Miltefosine, Oxaminiquine, Pyrimethamine and Sulfadiazine, Leucovorin, Amprolium, and Salinomycin.

### Pharmaceutical Compositions

The term effective amount, as used throughout, is defined as any amount necessary to produce a desired physiologic response, for example, treating or preventing a fungal infection, a bacterial infection, a viral infection, etc. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, unwanted cell death, and the like. Generally, the dosage will vary with the type of inhibitor, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary and can be administered in one dose or multiple doses administered daily or at extended intervals.

Any of the liposomes described herein can be provided in a composition, for example, a pharmaceutical composition. The composition can include one or more liposomes disclosed herein. Optionally, the composition comprising one or more liposomes is in a kit. Pharmaceutical compositions include, for example, a pharmaceutical composition comprising a therapeutically effective amount of any of the liposomes described herein and a pharmaceutical carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water.

Pharmaceutical compositions comprising any of the liposomes described herein can be prepared according to standard techniques and further comprise a pharmaceutically acceptable carrier. Generally, normal saline will be employed as the pharmaceutically acceptable carrier. Other suitable carriers include, e.g., water, buffered water, or saline. 0.4% saline. 0.3% glycine, dextrose, and the like, including glycoproteins for enhanced stability, such as albumin. lipoprotein, and globulin. These compositions are usually sterile. The pharmaceutical compositions can also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents. pH buffering substances, and the like, may be present in such vehicles. The preparation of pharmaceutically acceptable carriers, excipients and formulations containing these materials is described in, e.g., Remington: The Science and Practice of Pharmacy. 22nd edition. Loyd V. Allen et al. editors, Pharmaceutical Press (2012).

Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, and calcium chloride. Additionally, the liposome suspension may include lipid-protective agents which protect lipids against free-radical and lipid-peroxidative damages on storage. Lipophilic free-radical quenchers, such as alpha tocopherol and water-soluble iron-specific chelators, such as ferrioxamine, are suitable.

The concentration of the liposomes in the pharmaceutical formulations can vary widely. i.e., from less than about 0.05%, usually at or at least about 2-5% to as much as 10 to 30% by weight and will be selected primarily by fluid volumes, viscosities, in accordance with the particular mode of administration selected. Or the liposomes may be dried or lyophilized and resuspended to a desired concentration in water or buffers at time of use. The amount of liposomes or the amount of active agent in the liposome administered depends upon the particular label used, the disease state being diagnosed and the judgment of the clinician but is generally between about 0.01 and about 150 mg per kilogram of body weight, preferably between about 0.1 and about 20 mg/kg of body weight, about ( ) 1 to about 10 mg/kg of body weight or about 0.1 to about 5 mg/kg of body weight, which may be administered in a single dose or in the form of individual doses, such as from 1 to 4 times per day. Administration can be performed for 1, 2, 3, 4, 5, 6, 7, 8, 9, 10, 11, 12, 13, 14, 15, 16, 17, 18, 19, 20 or more days. One of skill in the art would adjust the dosage as described below based on specific characteristics of the agent and the subject receiving it.

The compositions disclosed herein are administered in a number of ways depending on whether local or systemic treatment is desired, and on the area to be treated. The compositions are administered via any of several routes of administration, including orally, intranasally, via inhalation, via nebulizer, parenterally, intravenously, intraperitoneally, intracranially, intraspinally, intrathecally, intraventricularly, intramuscularly, subcutaneously, intracavity or transdermally. Pharmaceutical compositions can also be delivered locally to the area in need of treatment, for example by topical application or local injection. The pharmaceutical compositions can also be delivered via pump or at a surgical site. Effective doses for any of the administration methods described herein can be extrapolated from dose-response curves derived from in vitro or animal model test systems.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Example I

The following examples are provided by way of illustration only and not by way of limitation. Those of skill in the art will readily recognize a variety of non-critical parameters that could be changed or modified to yield essentially the same or similar results.

Human DC-SIGN (a.k.a., DC-SIGN, CD-SIGN, ICAM-3) is C-type lectin pathogen receptor encoded by the CD209 gene. Its carbohydrate recognition domain (CRD) binds variously crosslinked mannose-rich and fucosylated glycans (e.g., the Lewis^{X} trisaccharide), and lipomannans often found in protein conjugates (Holla et al. “Comparative analysis reveals selective recognition of glycans by the dendritic cell receptors DC-SIGN and Langerin,” Protein Engineering. Design and Selection 24:659-669 (2011)). DC-SIGN is expressed by many classes of dendritic cells and phagocytes and a few other cell types. After dendritic cell's DC-SIGN's extracellular CRD binds to glycans expressed by a pathogen, its N-terminal cytoplasmic domain signals both innate and adaptive immune responses to infection. Seemingly at odds with stimulating a defensive immune response, DC-SIGN binding to a fungal pathogen can also mediate fungal cell uptake by dendritic cells and thereby promote infection (Cambi et al. “Dendritic cell interaction with Candida albicans critically depends on N-linked mannan. The Journal of biological chemistry 283:20590-20599 (2008)). Because mice encode eight genetic homologs of DC-SIGN and have no clear DC-SIGN ortholog, the experiments described herein were conducted with human DC-SIGN.

Among several fungal pathogens recognized by DC-SIGN, three highly divergent genera, Aspergillus spp., Candida spp., and Cryptococcus spp., cause a majority of global life-threatening invasive fungal infections and hundreds of thousands of deaths annually. They are partially representative of the extreme diversity in the fungal kingdom, because the three phyla that they represent are estimated to have common ancestry from 0.8 to 1.3 billion years ago. These three fungi are responsible for approximately 4.5 billion dollars in U.S. medical costs annually further supporting their importance as models for the studies presented herein. As described below, DC-SIGN targeting of a liposomal antifungal drug to these three fungal species was examined.

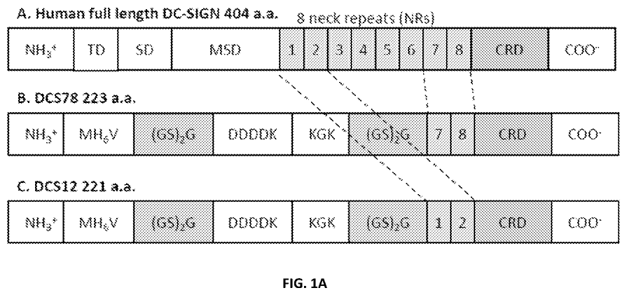

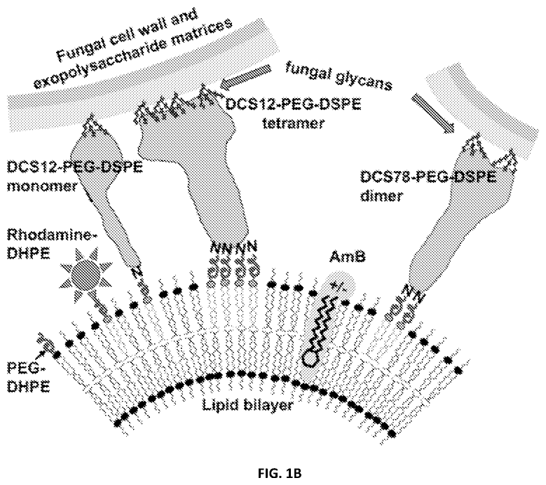

Full length human DC-SIGN (NCBI accession #Q9NNX6.1) is a 404 amino acid (a.a.) long protein (FIG. 1). The protein starts with a N-terminal ER signal sequence, followed by a short cytoplasmic signaling domain, a single pass membrane spanning domain, a long neck region comprised of 8 sequence-related 21 to 23 amino acid (a.a.) long neck repeats, presented sequentially as NR1 to NR8, and a C-terminal 151 a.a. CRD responsible for glycan binding (8, 33-35) (FIG. 1A). The sequences of these domains are annotated in FIG. 5. Alternate splicing produces a number of DC-SIGN isoforms that lack from one to seven of the eight NRs, retain the CRD and still bind glycan ligands (Serrano-Gómez et al., “Structural Requirements for Multimerization of the Pathogen Receptor Dendritic Cell-specific ICAM3-grabbing Non-integrin (CD209) on the Cell Surface,” Journal of Biological Chemistry 283:3889-3903 (2008)). The various isoforms of DC-SIGN float in the cell membrane as monomers, dimers, and tetramers and all three forms bind glycan ligands. Different combinations of NRs stabilize distinct homo-multimers which presumably have higher avidity than monomers and are proposed to provide alternate membrane positioning that may influence the specificity of glycan binding on a pathogen's surface. As shown herein, recombinant isoforms of DC-SIGN selectively target antifungal drug loaded liposomes to fungal pathogens and one exemplary isoform has enhanced antifungal activity relative to untargeted antifungal liposomes.

### Strains of Fungi and Media

Three fungal species were examined herein (1) C. albicans strain CA14, expressing GFP under control of the ADH1 promoter (Keppler-Ross et al. “Recognition of yeast by murine macrophages requires mannan but not glucan,” Eukaryot Cell 9:1776-87 (2010)) and was derived from a human isolate (SC5314, ATCC MYA-2876) deleted for URA3 (strain CA14, δura3::imm434/δura3::434) (Fonzi et al. “Isogenic strain construction and gene mapping in Candida albicans,” Genetics 134:717-28 (1993)), (2) A. fumigatus A1163 (3), and (3) wild type C. neoformans H99-alpha (Montone et al. “Regulating the T-cell immune response toward the H99 strain of Cryptococcus neoformans,” Am J Pathol 175:2255-6 (2009)). A. fumigatus was grown in Vogel's Minimal Media (VMM)+1% glucose (62)+0.5% BSA at 37° C., C. albicans in RPMI 1640 media with no red indicator dye (ThermoFisher SKU-11835-030)+0.5% BSA or 10% fetal bovine serum (Life Technologies GIBCO #16170-078) at 37° C. and C neoformans in YPD (1% yeast extract, 2% peptone, 2% dextrose) at 30° C. in liquid media with shaking or on the surface of polystyrene microtiter plates and incubated for 6 to 36 hr.

### Mice and Immunosuppression

Seven-to eight-week-old outbred female CDI (CD-1 IGS) Swiss mice (27 g to 30 g ea.) were obtained from Charles River Labs. Mice were maintained in UGA's Animal Care Facility. All mouse protocols met guidelines for the ethical treatment of non-human animals outlined by the U.S. Federal government and approved by UGA's Institutional Animal Care and Use Committee (AUP #A2019 08-031-A1). Immunosuppressed neutropenic mice were obtained by treating with both the antimetabolite cyclophosphamide (CP, Cayman #13849, Ann Arbor MI) and the synthetic steroid triamcinolone (TC, Millipore Sigma #T6376, Burlington, MA) three days prior to infection. CP and TC stocks, dilutions, and injection methods were described recently (Ambati et al. mBio 12:1-8 (2021)). Infected animals not receiving antifungal therapy and many of the AmB-LL treated mice first showed a ruffled coat due to reduced grooming and a few showed severe lethargy. Mice were sacrificed by cervical dislocation following anesthesia with isoflurane (Animal Use Protocol, A2019 08-031-A1).

Producing DCS12 and DCS78 Isoforms of DC-SIGN and Integrating them into Drug Loaded Liposomes

AmB-LLs were prepared as described previously (Ambati et al. mSphere 4:1-15 (2019)). AmB-LLs are pegylated analogs of commercial AmBisome R, with an equivalent amount of Amphotericin B, 11 moles percent relative to liposomal lipid. Pegylation protects liposomes from opsonization and phagocytosis, which extends the half-life of packaged drug

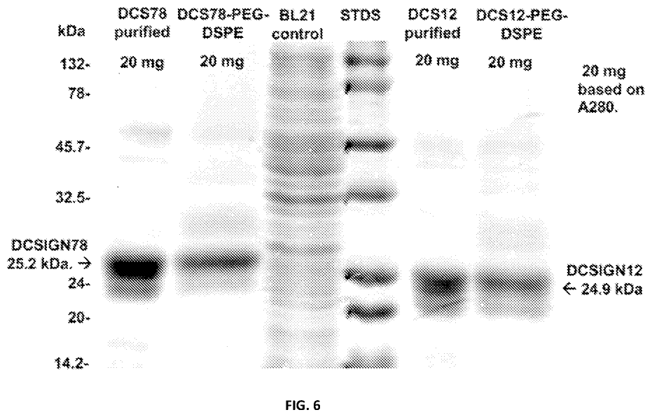

C-type lectin receptors such as DC-SIGN are membrane proteins and their carboxyterminal extra cellular CRDs are hydrophobic and therefore relatively insoluble in normal biological buffers. Soluble functional isoforms are inefficiently recovered from E. coli. For example, it was reported that only 0.5 to 0.7 mg of soluble DC-SIGN was recovered from a liter of isopropyl β-D-1-thiogalactopyranoside (IPTG) induced culture in one of the best previous studies (Pederson et al. Structural characterization of the DC-SIGN-Lewis (X) complex. Biochemistry 53:5700-5709 (2014)). The a.a. sequences of the native human DC-SIGN and the two recombinant proteins used herein are shown in FIG. 5. The purification protocol was essentially as described in Ambati et al. (2019), as summarized briefly here. E. coli optimized DNA encoding sequences were cloned into expression plasmid pET-45b+ and in E. coli BL21. Following 4 hr of induction with IPTG, the insoluble protein was extracted from cell pellets in 6 M Guanidine Hydrochloride and affinity purified on nickel affinity resin. Approximately 40 mg of 75 to 80% pure DCS12 and DCS78 protein were recovered (FIG. 6). While still in a 6 M GuHCl buffer. DCS12 and DCS78 were coupled with the lipid carrier DSPE-PEG-3400-NHS. Gel exclusion chromatography over P6 acrylamide resin was used to remove GuHCl and excess coupling agent buffer exchange the protein into a 1 M arginine crowding buffer with beta-mercaptoethanol (BME). DCS12-PEG-DSPE and DCS78-PEG-DSPE (FIG. 6) remain soluble in this buffer and were integrated via their DSPE lipid moieties into the phospholipid bilayer membrane of AmB-LL at 1.0 moles percent of DC-SIGN CRD relative to moles of liposomal lipid to make DCS12-AmB-LLs and DCS78-AmB-LLs. As controls bovine serum albumin coated liposomes. BSA-AmB-LLs, were made, which along with uncoated parent AmB-LLs, served as controls. Lissamine rhodamine B-DHPE was simultaneously incorporated at two moles percent in all liposome preparations (FIG. 1B). allowing sensitive and quantitative comparisons of fluorescence to estimate the binding efficiency of all three liposomal preparations to cells expressing appropriate ligands. All liposome stocks were stored in the IM arginine RN5 buffer such that the AmB concentration was 600 to 800 μM. Fresh BME was added to 2 mM every month during storage. Just prior to their use. DCS12 and DCS78 liposomes were diluted first 10-fold into liposome dilution buffer #2 (LDB2. 20 mM HEPES. 10 mM Triethanolamine. 150) mM NaCl. 2 mM CaCl₂). 0.5% BSA pH 8.0- and 0.7-mM beta-mercaptoethanol (BME), where the BME was added fresh.

### Microscopy and Quantification of Liposome Binding to Fungal Cells

Cells were grown in 24-well microtiter plates, washed thrice with PBS, fixed in 4% formaldehyde in PBS for 60 mins. washed thrice, and stored at 4° C. in PBS. Fixed fungal cells pre-incubated for 30 to 60 min with in LDB2+5% BSA at 23° C. Liposomal stocks were diluted into LDB2+5% BSA before incubating with cells such that the DCS12 or DCS78 liposomal protein concentration was 1.0 ug/100 uL. After 1 hr incubation at 23° C. unbound liposomes were washed out with 4 changes of LDB2+5% BSA. Ten separate fluorescent images of rhodamine red fluorescent liposomes bound to cells grown on 24 well microtiter plates, were taken at 10× or 20× magnification on an Olympus inverted microscope (Model IX70) with a digital camera attached. The area of red fluorescent liposome binding from 10 random images was quantified by taking an 8-bit grey-scale copy of the unmodified red fluorescent JPEG image into Image J (imagej.nih.gov/ij), as described in Ambati et al. mSphere 4:1-16 (2019)). The green channel of bright field images showing fungal cells and red fluorescent images of liposomes were merged in Photoshop for presentation.

### Growth Inhibition and Viability Assays Following Liposome Treatment

Cell viability and metabolic activity after AmB loaded liposome treatment was assayed by a protocol modified from that described previously in Ambati et al. (2019). C. albicans were seeded at 4,000 cells per well unless noted otherwise in 100 uL RPMI+10% FBS into 96 well microtiter plates and grown for 7 hr at 37° C. until late germling to early hyphal stage. The media was replaced. Liposomal stocks used in growth inhibition assays were freshly diluted into LDB2 buffer+0.5% BSA and then diluted 1:11 into growth media to achieve the indicated final AmB concentrations ranging from 3 μM down to 0.025 μM. Control cells received an equivalent amount of buffer. After 1 hr incubation at 37° C. the plates were gently agitated, and unbound liposomes were removed without further washing. 100 μL of fresh growth media was added. After 4.5 hr incubation at 37° C., 20 μL of CellTiter-Blue (CTB) reagent was added per well as per the manufacturer's instructions. After 1.5 hr incubation at 37° C. the fluorescent product produced by the reduction of CTB reagent was analyzed in a Bio-Tek Synergy HT fluorescent microtiter plate reader (ex485/em590). The fluorescent background from control wells with reagent, but no cells, was subtracted from readings of experimental wells. Data from six wells were averaged for each data point.

### Fungal Burden Estimates

Fungal burden in excised kidney pairs from infected animals was estimated on day 1 post-infection by assaying both the number of CFUs and the amount of C. albicans ribosomal rDNA intergenic transcribed spacer (ITS) using quantitative real-time PCR (qPCR). Kidney pairs were weighed and minced into hundreds of approximately 1 mm³ pieces, the pieces mixed to account for the uneven distribution of infection centers, and aliquoted into 25 mg samples. 1. CFUs. 25 mg of the minced kidney tissue was homogenized for 60 seconds in 200 μL of PBS using a hand-held battery powered homogenizer (Kimble, cat #749540-0000) and blue plastic pestle (Kimble Cat #749521-1500). The homogenate was spread evenly by shaking with sterile glass beads on 5 mm thick YPD agar plates containing 100 μg/mL each of Kanamycin and Ampicillin. After a 11 hr incubation at 37° C., the microcolonies were counted for ten fields of view on an EVOS imaging system (AMG Fl) at 4× magnification. The number of CFUs was corrected for the area of the entire plate relative to each microscopic field and the weight of each kidney pair. 2. qPCR. DNA was extracted from parallel 25 mg samples of kidney homogenates using Qiagen's DNeasy® Blood & Tissue Kit (#69504) protocol modified as described in Ambati et al. (2021). QPCR was used to estimate the relative amount of C. albicans rDNA ITS sequence in 100 ng samples of infected kidney DNA using the conditions described in Ambati et al. (2021), and appropriate primer pairs The Relative Quantity (RQ) of C. albicans rDNA ITS was determined by normalizing all Ct values to the lowest Ct value determined for an infected control kidney using the dCt method (Livak et al. “Analysis of relative gene expression data using real-time quantitative PCR and the 2 (-Delta Delta C(T)) Method,” Methods 25:402-408 (2001)).

### Data Management

Data were recorded and managed in Excel (v. 16.16.27). Because most of data were reasonably normally distributed, the student's two-tailed t test, T.TEST in Excel, was used to estimate P values. Scatter bar plots were prepared, and standard errors estimated in Graph Pad Prism 9 (v. 9.0.0).

### RESULTS

### DC-SIGN Targeting of Liposomes

Two truncated isoforms of human DC-SIGN, each containing the 151 amino acid (a.a.) long CRD, two NRs, a Lys-Gly-Lys peptide for coupling to a lipid carrier, a potential protease processing site, and a hexa-histidine tag for protein purification, with each of the last three domains separated by short flexible Gly-Ser-Gly spacers (FIGS. 1B and 1C), were designed. The DCS78 construct has NR7 and NR8 fused to the CRD, preserving the natural linkage of these two NR to the CRD (FIG. 1B, FIG. 5B). All the known natural splice variants with NR7 and NR8 include a few other NR, but none have been reported with only NR7 and NR8 fused to the CRD. It was expected that DCS78 would retain ligand binding specificity based on the activity of other longer isoforms and the fact that NR7 and NR8 are adjacent to the CRD in the native gene structure. The second construct, DCS12, has distal NR1 and NR2 fused to the CRD (FIG. 1C). The design of DCS 12 is based on a known splice-variant of DC-SIGN that efficiently forms functional homotetramers and recognizes C. albicans yeast cells, mannan-agarose, and N-Acetyl Galactosamine-agarose. However, the DCS12 construct used herein lacks the membrane and signaling domains retained in the native isoform. Using Expasy P (https://web.expasy.org/protparam/), it was predicted that DCS12 and DC78 would be unstable hydrophobic proteins with high percentage of aromatic a.a. residues. The expression and renaturation technology described in Ambati et al. (2019) and in the Examples was used.

The pegylated AmB-LLs were loaded with 11 moles percent AmB relative to moles of liposomal lipid, analogous in this respect to commercial non-pegylated AmBisome®. Affinity purified denatured DCS12 and DCS78 proteins (FIG. 6), each of approximately 25 kDa, were coupled via a Lys residue to the reactive NHS group of the lipid carrier NHS-PEG-DSPE. The partially renatured protein conjugates were inserted via their lipid DSPE moieties into the membrane of AmB-LLs at a concentration of one mole percent polypeptide relative to moles of liposomal lipid, to make separate DCS12-AmB-LLs and DCS78-AmB-LLs. FIG. 1D illustrates the way in which DCS12 and DCS78 are expected to be multimerized on the surface of DCS12-AmB-LLs and DCS78-AmB-LLs. Bovine Serum Albumin (65 kDa) coated BSA-AmB-LLs with 0.35 moles percent BSA were also constructed as protein coated untargeted liposome controls. Two moles percent of DHPE-Rhodamine B was also inserted into the liposome membrane to fluorescently tag all four types of liposomes, AmB-LLs, DCS12, DCS78, and BSA-AmB-LLs. The final chemical compositions of DCS12 and DCS78 liposomes in comparison to AmBisome®

TABLE 2
Liposome composition compared to that of Gilead's AmBisome.¹

| | Gilead Commercial | AmB-LLs | DCS12-AmB-LLs or |
|---|---|---|---|
| Compounds | AmBisome ® | herein | DCS78-AmB-LLs |
| Additions to liposomes conjugated to | Moles percent of | Moles percent of | Moles percent of |
| lipid carriers in this manuscript | additions to base | additions to base | additions to base |
| | liposomes | liposomes | liposomes |
| DCS12 or DCS78 | 0.0 | 0.0 | 1.0 |
| Amphotericin B | 10.6 | 11.0 | 11.0 |
| Lissamine Rhodamine-PE | 0.00 | 2.0 | 2.0 |
| [symbol] 100% | | | |
| alpha-Tocopherol (form of Vitamine E) | 0.0003 | 0.0 | 0.0 |
| mPEG2000-DSPE (N-(Carbonyl- | 0.0 | 5.0 | 5.0 |
| methoxypolyethylene glycol 2000)-distearoyl- | | | |
| glycerophosphoethanolamine) | | | |
| HSPC (soy phosphotidylcholaine) | 52.7 | 0.0 | 0.0 |
| [symbol] | | 50.0 | 50.0 |
| phosphocholine) | | | |
| phosphoglycerol) | 21.1 | 0.0 | 0.0 |
| CHOL (Chlosterol) | 26.2 | 45.0 | 45.0 |
| Total unconjugated lipid = 100% | 100 | 100.0 | 100.0 |

¹Gilead, S. AmBisome ® (amphotericin B) liposome for injection. 1-27 (2012).
https://www.astellas.us/docs/ambisome.pdf [symbol]
indicates data missing or illegible when filed

### DCS12 and DCS78 Coated Liposomes Bound to Fungal Exopolysaccharide Matrices

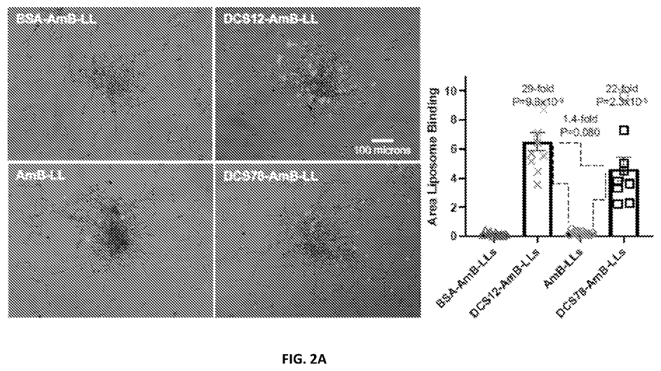

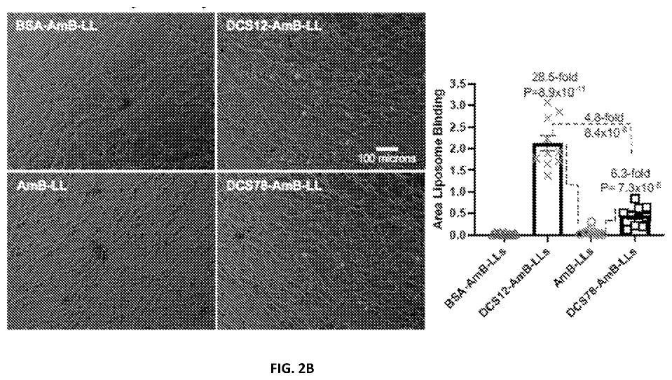

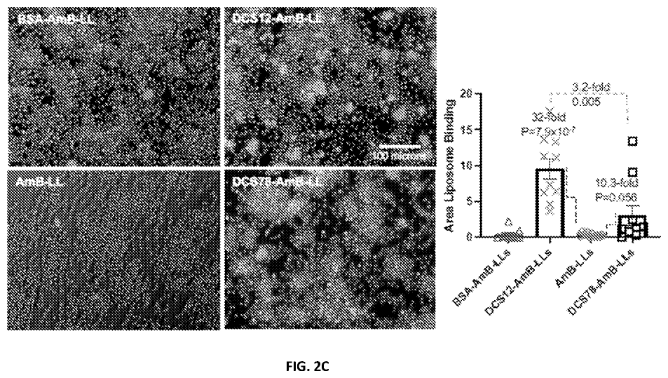

The ability of DCS12-AmB-LLs and DCS78-AmB-LLs to bind in vitro grown fungal cells relative to AmB-LLs and BSA-AmB-LLs was quantified by measuring the area of red fluorescent liposome binding to fungal cells in ten photographic images (FIG. 2). DCS12-AmB-LLs bound 29-fold more strongly to what appear to be regions of secreted exopolysaccharide matrix surrounding colonies of C. albicans hyphae than AmB-LLS (P=9.8×10⁻⁹) and only slightly more strongly than DCS78-AmB-LLs (FIG. 2A). Patches of exopolysaccharide binding appeared more frequent at the periphery of colonies than at the center. DCS12-AmB-LLs bound 28.5-fold more strongly to patches of exopolysaccharide associated with A. fumigatus hyphae than AmB-LLS (P=8.4×10⁻¹¹) and 4.8-fold more strongly than DCS78-AmB-LLs (P=8.4⁻⁸) (FIG. 2B). DCS12-AmB-LLs bound 32-fold more strongly to the exopolysaccharide matrices surrounding C. neoformans than AmB-LLs (P=7.9×10⁻⁷) and 3.2-fold more strongly than DCS78-AmB-LLs (P=0.005) (FIG. 2C). BSA-AmB-LLs did not bind significantly to any of the three fungal species. Although we can't exclude low levels of cell wall binding, both targeted liposomes did not appear directly associated with fungal cell walls. Because DCS12 bound to all three species more than an order of magnitude more efficiently than untargeted AmB-LLs or BSA-AmB-LLs and 1.4-to 4.8-fold more efficiently than DCS78, DCS78 was dropped from further analysis.

DCS12-AmB-LLs Efficiently Inhibited or Killed C. albicans Cells In Vitro

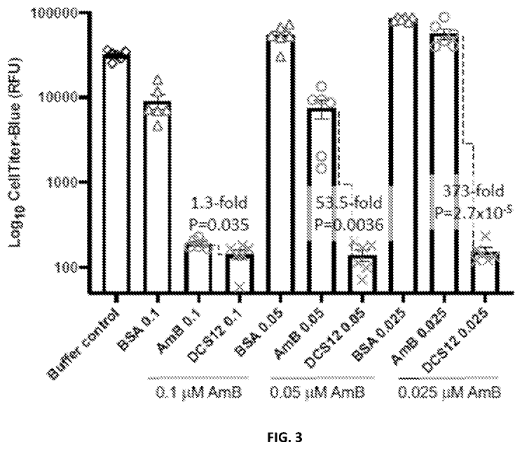

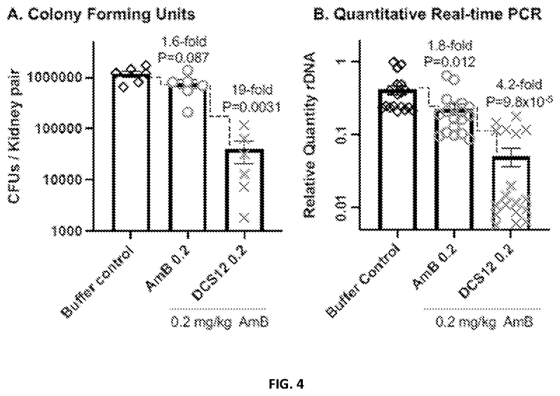

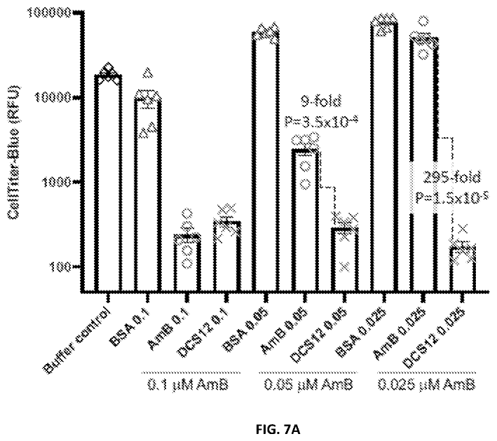

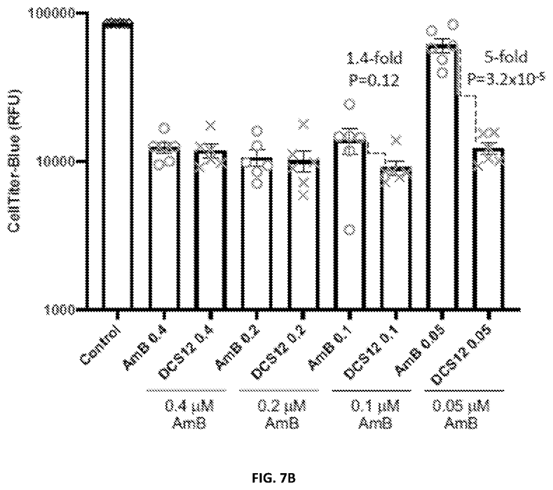

Microtiter plates were seeded with 4.000 C. albicans cells per well. After cells were grown for 7 hours and had reached the late germling and early hyphal stage, they were treated for 60 min with AmB-LLs. BSA-AmB-LLs and DCS12-AmB-LLs delivering a range of AmB concentrations. The growth medium with unbound liposomes was then removed without additional washes and replaced with fresh growth medium. Four and a half hours later. CellTiter-Blue (CTB) reagent was added and cells were incubated for 1 hr. CTB assays measure viability based on the redox activity of electron transport linked mitochondrial reductases active in living cells generating a fluorescent product, resorufin, which we then quantified. FIG. 3 shows that targeted DCS12-AmB-LLs delivering 0.05 and 0.025 μM AmB killed or inhibited the activity of C. albicans cells 53-fold (P=0.0036) and 373-fold (P=2.7×10⁻⁵), respectively, more efficiently than uncoated AmB-LLs delivering the same concentrations of AmB. When delivering 0.1 μM AmB. AmB-LLs were also very effective at reducing cellular metabolic activity and only slightly less effective than DCS12-AmB-LLs (P=0.035). BSA-AmB-LLs were much less effective than either AmB-LLs or DCS12-AmB-LLs at these concentrations. Repetitions of this experiment gave very similar results (FIG. 7). It is likely that the relatively low metabolic activity of cells in the buffer treated control samples (FIG. 3 & FIG. 7, left) results from their having reached stationary phase some time before the CTB assay was performed or that the fluorescent resorufin was further reduced to non-fluorescent hydroresorufin. At higher AmB concentrations of 0.2 and 0.4 μM, the improved in vitro performance of DCS12-AmB-LLs over AmB-LLs could not be resolved. (FIG. 7B).

DCS12-AmB-LL Reduced the Fungal Burden in the Kidneys of Mice with Candidiasis

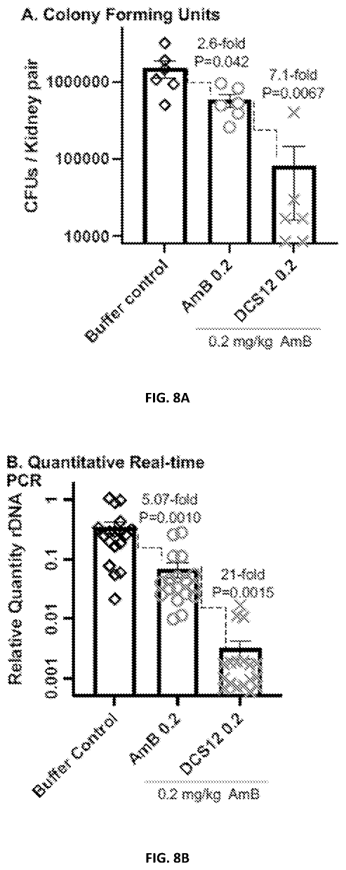

Mice rendered neutropenic by treatment with cyclophosphamide (CP) and triamcinolone (TC) were infected intravenously with 7.5×10⁶ C. albicans yeast cells. Three to four hr post infection they were given an intravenous injection of AmB-LLs or DCS12-AmB-LLs delivering 0.2 mg/kg AmB diluted into liposome dilution buffer 2 (LDB2) or with the same amount of LDB2 alone (Buffer control). Twenty-four hr post infection, the mice were sacrificed, their kidneys excised, homogenized, and assayed for fungal burden. Mice treated with DCS12-AmB-LLs showed a significant 19-fold lower average number of CFUs per kidney pair relative to AmB-LL treated mice (P=0.0031, FIG. 3) and a 4.2-fold lower fungal burden in the kidneys based on quantitative real time qPCR assays of the amount of fungal rDNA ITS (P=9.8×10⁻⁵, FIG. 3B). At 0.2 mg/kg AmB-LLs produces a slight drop in fungal burden relative to Buffer treated controls. Repetitions of this experiment gave similar results (FIG. 8). In summary, in a neutropenic mouse model of invasive candidiasis, DC-SIGN targeting significantly improved the efficacy of liposomal AmB for reducing fungal burden in the kidneys.

As shown herein, exemplary DC-SIGN constructs, DCS12 and DCS78 bound to three diverse fungal species, with DSC12 exhibiting superior binding properties. As such, although DC-SIGN targeting of pathogens is not limited to a specific DC-SIGN construct. DCS12 could be a pan-fungal targeting protein not only because of its superior binding, but also because it is a natural isoform arrangement of NRs and CRD. This latter property is likely to reduce the immunogenicity of DCS12 when used clinically.

The minimum inhibitor concentrations (MIC₅₀) for liposomal AmB (AmB-LLs/AmBisome R) against Candida spp. growing in vitro, defined herein as at least a 50% reduction in metabolic activity or CFUs, varies modestly among publications and experimental designs. MIC₅₀ values are typically 4 to 5 μM (3.7 to 4.6 mg/L) in vitro (Jullien et al. “Study of the effects of liposomal amphotericin B on Candida albicans, Cryptococcus neoformans, and erythrocytes by using small unilamellar vesicles prepared from saturated phospholipids.” Antimicrob Agents Chemother 33:345-9 (1989)): 1 to 7 mg/kg for one treatment in immune-suppressed mouse models (van Etten et al. “Biodistribution of liposomal amphotericin B (AmBisome) and amphotericin B-deoxycholate (Fungizone) in uninfected immunocompetent mice and leucopenic mice infected with Candida albicans,” J Antimicrob Chemother 35:509-19 (1995)), and 3 to 5 mg/kg/day for several days in the clinic (Pea et al. “Overview of antifungal dosing in invasive candidiasis,” J Antimicrob Chemother 73:133-143 (2018)). Surprisingly, as shown herein, lower doses of AmB delivered by DCS12-AmB-LLs, 0.025 UM for in vitro grown cells and a single dose of 0.2 mg/kg in vivo in an immunosuppressed mouse model, significantly improved the performance of liposomal AmB against C. albicans. Lower effective doses of AmB could translate into greater efficacy and reduced toxicity in the clinic. A wide range in the effectiveness of DCS12-AmB-LLs to reduce fungal burden among individual mice was observed. Among the potential causes for this variability are differences in the levels of infection observed even in control mice, differences in the effective delivery of DCS12-AmB-LLs liposomes to infected tissues, and differences in the expression of glycan targets to which DCS12-AmB-LLs bind. In some instances, a regimen with multiple treatments, as are often used for AmB-LLs, would improve the performance of DC-SIGN targeted liposomes.

Based on these data, it is clear that DCS12 bound efficiently to the exopolysaccharide matrices associated with C. albicans, A. fumigatus, and C. neoformans grown in vitro. DCS12-AmB-LLs were more effective at inhibiting and/or killing C. albicans grown in vitro. In an in vivo neutropenic mouse model of candidiasis, DCS12 was more effective at reducing fungal burden in the kidneys than untargeted AmB-LLs or BSA coated BSA-AmB-LLs.

Considering that DCS12-AmB-LLs bound efficiently to three divergent fungal species and how well DC-SIGN targeting improved the performance of liposomal AmB against C. albicans, it is reasonable to propose that DC-SIGN coated liposomes might effectively deliver cytotoxic drugs to life-threatening pathogens in other kingdoms that are also recognized by DC-SIGN. For example, DC-SIGN binds surface carbohydrates expressed by the bacterium Mycobacterium tuberculosis, the animal helminth Schistosoma mansoni, and the protist Leishmania, to name a few. Respectively, these three pathogens cause millions, hundreds of thousands, and tens of thousands of deaths annually and drug treatment failures are common for all three (Singh et al. “Recent updates on drug resistance in Mycobacterium tuberculosis,” J Appl Microbiol 128:1547-1567 (2020): Verjee et al. “Schistosomiasis: Still a Cause of Significant Morbidity and Mortality,” Research and reports in tropical medicine 10:153-163 (2019)). DC-SIGN targeting might improve the performance of existing and new cytotoxic drugs used to treat these pathogens. It is worth noting that AmBisome® has been an FDA approved treatment for Leishmaniasis, since 1997 (Meyerhoff “U.S. Food and Drug Administration approval of AmBisome (liposomal amphotericin B) for treatment of visceral leishmaniasis,” Clin Infect Dis 28:42-8: discussion 49-51 (1999)).

As shown herein. DC-SIGN constructs can be used to effectively target pathogens for treatment of pathogenic infection. Two exemplary recombinant isoforms combining the CRD with NR1 and NR2 (DCS12) or with NR7 and NR8 (DCS78) were prepared and coupled to a lipid carrier. These were inserted into the membrane of AmB-LLs to construct DCS12-AmB-LLs and DCS78-AmB-LLs. Relative to AmB-LLs, DCS12-AmB-LLs and DCS78-AmB-LLs bound efficiently to the exopolysaccharide matrices produced by A. fumigatus, C. albicans and C. neoformans in vitro, with DCS12-AmB-LLs performing better than DCS78. Relative to AmB-LLs. DCS12-AmB-LLs delivering only 0.025 μM AmB inhibited or killed C. albicans in vitro 300-fold more effectively and delivering 0.2 mg/kg AmB reduced the kidney fungal burden several-fold in a mouse model of candidiasis. These data suggest DC-SIGN targeting of liposomal antibiotics has the potential to alter antimicrobial treatment paradigms for a wide variety of pathogens.

### Example II

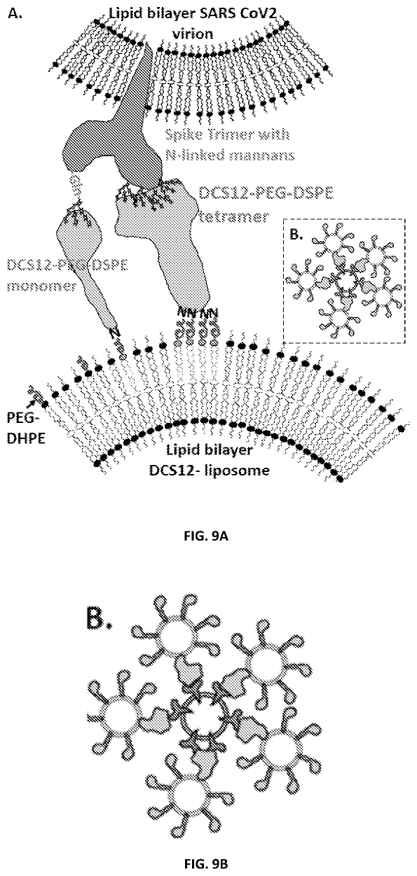

DC-SIGN recognizes glycans linked to proteins on the surface of evolutionarily diverse classes of RNA and DNA viruses. These include, but are not limited to, SARS coronaviruses, Influenza virus, HIV1, HIV2, Ebola Virus, Dengue virus, Herpes simplex virus 1, West-Nile virus, and Measles virus. Liposomes coated with large numbers of DC-SIGN's CRD could have great avidity for binding to virions of the above-mentioned viruses (FIG. 9A). As a particular example, coronaviruses SARS COV and SARS COV2 have several conserved N-mannosylation sites on their Spike proteins. Approximately 25% of SARS COV2 Spike's 730 a.a., long ectodomain's glycan shield is composed of mannans linked to these sites that are known cognate ligands of DC-SIGN (e.g., Man5GlcNAC2, Man6GlcNAC2, Man7GlcNAC2, Man8GlcNAC2, and Man9GlcNAC2 16). DC-SIGN liposomes could bind with high avidity because multiple DC-SIGN CDRs would bind concurrently to multiple viral ligands on the surface of a single virion and to multiple ligands on a single Spike protein (FIG. 9A). Therefore, liposomes coated with DC-SIGN's CRD will bind tightly all over the surface of virions (FIG. 9B), sequester virions, and interrupt their infection cycle. DC-SIGN coated liposomes will bind with high avidity to diverse classes of viruses including SARS COV2 and bind to a more diverse set of ligands than any one antibody, and like antibodies it will then disrupt viral replication cycles. Therefore, DC-SIGN liposomes could be used as a pan-antiviral drug.

### Example III

### Dectin-Targeted Nanoparticles for Inhibition of Bacterial Infection

M. tuberculosis is the primary cause of human tuberculosis (TB) and, with very few exceptions, has been the annual leading cause of death from infectious disease for centuries. About one-quarter of the world's population may have latent or active infections. Globally, every year there are approximately 10 million new cases and 1.7 million deaths from TB. M. ulcerans is a true pathogen, which causes debilitating skin lesions. M. avium is an opportunistic pathogen that causes life threatening pulmonary infections primarily in immunocompromised individuals. Unlike most bacterial pathogens, M. tuberculosis and NTM pathogens require extended multidrug antibiotic treatments of six months or more to clear infection and prevent relapse. But extended antibiotic therapies, in addition to being expensive and problematic to maintain, produce a range of adverse effects such as gastrointestinal disturbances, liver toxicity, and/or peripheral neuropathy. Furthermore, the incidence of multidrug resistant TB- and NTM-related mortality is rapidly increasing, and successful treatment with second line drugs may take up to 20 months. There is a demand for new drug delivery systems that will provide increases in anti-mycobacterial drug efficacy and/or reduce treatment regimen times.

Pathogenic mycobacteria, including M. tuberculosis, M. avium, and M. ulcerans have thick cell walls rich in lipids, mycolic acids, peptidoglycans, and mannose-capped lipoarabinomannan (ManLAM) and produce even more extensive exopolysaccharide matrices and biofilms rich in oligoglucan-, oligomannan-, and lipoglycan-containing polysaccharides. The mycobacterial capsule and extracellular matrix often represent the greater part of their biomass. We hoped to take advantage of this extensive exopolysaccharide layer to develop a novel antibacterial delivery system targeting drug loaded liposomes to mycobacteria. But first, it was necessary to identify targeting proteins that efficiently bound nanoparticles to pathogenic mycobacteria.

### Mycobacterial Strains and Growth

M. tuberculosis strain Erdman K01 (TMC 107) was procured from BEI Resources. Stocks were cultured in Proskauer Beck medium as pellicles under BSL3 biological containment. M. ulcerans strain S-WT was obtained from the reference culture collection at the Centers for Disease Control and Prevention (Atlanta, GA) and grown in Middlebrook 7H9 broth supplemented with albumin, dextrose, and catalase and 0.2% glycerol. M. avium serotype 4 strain was modified by the genome integration of a green fluorescent protein GFP gene under control of the hsp60 promoter to make M. avium (hsp60 gfp). M. avium was also cultured in supplemented Middlebrook 7H9 broth. M. avium and M. ulcerans were grown under BSL2 containment. All protocols followed the University of Georgia's institutional guidelines.

### Fluorophores, Liposomes, and Liposome Buffers for Staining Cells

All blocking and staining protocols were performed in liposome dilution buffers LDB1 for Dectin-1 (Dulbecco's phosphate buffered saline (PBS), pH 7.2, 5% BSA, 1 mM 2-mercaptoethanol) and LDB2 for Dectin-2 and DC-SIGN (20 mM HEPES, 10 mM Triethanolamine, 150 mM NaCl, 10 mM CaCl₂, pH 8.0+5% BSA+1 mM fresh BME). A 25 mM stock of Calcofluor white (CW) (Blankophor BBH SV-2560, Bayer, Corp., CAS 16090 Feb. 1) was prepared as a fine suspension in di-water with 5% DMSO and stored at 4° C. After warming to room temperature, the stock was diluted to a final concentration of 25 μM in LDB1 or LDB2 for staining cells.

### Liposomes

Pegylated 100 nanometer diameter liposomes were obtained from FormuMax Sci. Inc. (Sunnyvale, CA) (F20203, DSPC: CHOL: mPEG-DSPE, 50:45:5 mol/mol ratio). Proteins to be used to coat the liposomes were modified at available lysine residues with the lipid carrier NHS-PEG-DSPE as described previously (Ambati et al. Fungal Biol. and Biotech 8:1-13 (2021): Ambati et al. mSphere 4:1-15 (2019): Ambati et al. mSphere 4:1-16 (2019)). Lissamine rhodamine B-DHPE was obtained from Invitrogen (L1392) (Waltham, MA). Proteins and Rhodamine B were inserted quantitatively into the liposomal lipid bilayer via their respective lipid moieties, DSPE and DHPE, at the transition temperature of these liposomes, 60° C. The predicted composition of the liposomes employed is shown in Table 3. All liposome preparations were stored at a concentration of approximately 6.25 nmoles liposomal lipid per mL and those coated with protein had approximately 1,400 ug of protein per mL. Liposomes were diluted 1:140 into LDB2 during cell staining, except for the polysaccharide inhibition studies in which they were diluted 1:280, which resulted in the proteins being at approximately 1:100 w/v or 1:200 w/v, respectively, during staining. The chemical analog of CW that was employed efficiently stained nearly all M. avium and M. ulcerans cells examined. However, even faint CW staining of M. tuberculosis was not observed. Stocks of laminarin (Sigma Cat #L-9634) and yeast oligomannan (Sigma, M3640) (St. Louis, MO) to be used in inhibition studies were prepared as 10 mg/mL in dd-H20. Liposomes (1:200 w/v Dectin-1 or Dectin-2) were incubated in their appropriate dilution buffers containing 1.0 mg/mL laminarin or yeast oligomannan for 30 min with tumbling at 37° C. and then added to an equal volume of cells along with 25 μM CW to a final volume of 100 μL, and tumbled for an additional 60 min. The mixture was diluted 5-fold in buffer and sedimented for 2 min at 2000×g and the cell pellet washed 4 more times before being imaged by epifluorescence.

TABLE 3
Composition of Liposomes

| | | Estimated # | Estimated |
|---|---|---|---|
| | Protein coating | protein | Rhodamine B |
| Liposome name | (kilo-Dalton MW, | molecules | molecules |
| & Reference | kDa) | per liposome | per liposome |
| Rhod-L [5] | none | 0 | 3,000 |
| DEC1-Rhod-L [5] | Dectin-1's CRD | 1,500 | 3,000 |
| | and stalk region | | |
| | (22 kDa) | | |
| DEC2-Rhod-L [6] | Dectin-2's CRD | 1,500 | 3,000 |
| | and stalk region | | |
| | (22 kDa) | | |
| DCS12-Rhod-L [1] | DC-SIGN's CRD | 1,500 | 3,000 |
| | and neck repeats | | |
| | 1 and 2 (25 kDa) | | |
| BSA-Rhod-L [5] | Bovine Serum | 500 | 3,000 |
| | Albumin (66 kDa) | | |

### Photography and Image Processing

Mycobacterial cells and cell clusters were identified by epifluorescence using a GFP filter set (Ex480/Em527) for the GFP tag in M. avium or using a DAPI filter set (Ex360/Em470) for CW staining of M. ulcerans or by bright field imaging of M. tuberculosis. Epifluorescence images of rhodamine B red fluorescent liposomes (Ex560/Em645) were taken at 63× magnification on a Zeiss LSM 710 confocal laser scanning microscope or a Leica DM6000 fluorescence microscope or a Zeiss AXIO Imager MI fluorescence microscope or at 20×on an ECHO Resolve microscope. The red fluorescent liposomes produced very strong epifluorescence signals, and hence, exposure times of only 200 msec were used. The areas of red fluorescent liposome binding were estimated from 8 to 10 randomly selected fields of cells. The data were quantified by moving the unmodified fluorescent TIFF 8-bit RGB color images into Image J (imagej.nih.gov/ij, version 1.53a) using a method modified from that described previously (Ambati et al. 2019). Under Image>adjust>color balance the blue or green color of the cells was removed from the image leaving only the red liposome images. Under Image>type, the 8-bit grey scale was selected. Under image>adjust>threshold the threshold values were set to range from 30 to 255 to remove background fluorescence from all images. Under Analyze>measure the area of red fluorescent liposome binding was recorded in a table for quantitative presentation of the areas of binding. Some of the Rhod-L and BSA-Rhod-L-stained fields of cells showed zero red fluorescence. For these zero values, a value of 0.001 was substituted, which was the lowest experimental value measured in any experiment using the above-mentioned parameters in ImageJ. This allowed the fluorescent area data to presented in a log scale, which was necessary to show the orders of magnitude distribution data points in scatter bar plots. For the optimal presentation of microscope images in figures, the green or blue or bright field channels showing mycobacteria were enhanced in Photoshop (version 23.1.1), while the associated red channel showing liposomes required little if any enhancement.

### Data Management

Quantitative imaging data from ImageJ were managed first in Excel (v. 16.16.27). The data was then move to Graph Pad Prism 9 (v. 9.3.1), where scatter bar plots were prepared, and standard errors estimated. P values were estimated using the student's two-tailed t test, T.TEST in Excel for the data that were normally distributed. In those cases where the data for at least one sample in a comparison, typically the Rhod-L sample, appeared to be non-parametric in its distribution, P values were estimated using the Mann-Whitney test in Prism and were indicated as PMW values. Fold differences indicated in the figures represent differences in the average values.

The Design of Targeted Liposomes and their Potential to Bind Mycobacteria

Liposomes tagged with red fluorescent Rhodamine B (Rhod-Ls) were constructed and small batches were coated with equivalent milligram (mg) amounts of the CRDs of Dectin-1, Dectin-2, and DC-SIGN isoform DCS12, and with Bovine Serum Albumin (BSA), to produce DEC1-Rhod-Ls, DEC2-Rhod-Ls, DCS12-Rhod-Ls and BSA-Rhod-Ls, respectively as illustrated for a DEC1-Rhod-L in FIG. 10B. Their protein and rhodamine B content is summarized in Table 3 and shows very similar femtogram amounts of protein coating per liposome. Approximately 1,500 protein molecules had been incorporated into the outer lipid layer of each DEC1-Rhod-L, DEC2-Rhod-L, and DCS12-Rhod-L. Sensitive quantitative comparisons of the binding of the five liposome preparations to mycobacteria was possible because approximately 3,000 rhodamine B molecules had been incorporated into each AmB-LL and they were the precursors of all other liposomes.

Dectin-1- and Dectin-2-Targeted Red Fluorescent Liposomes Bound Efficiently to M. avium's Exopolysaccharide Matrix

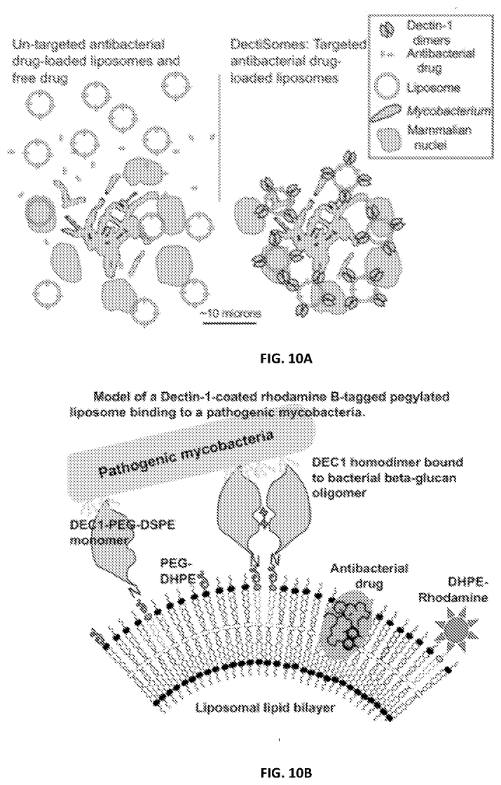

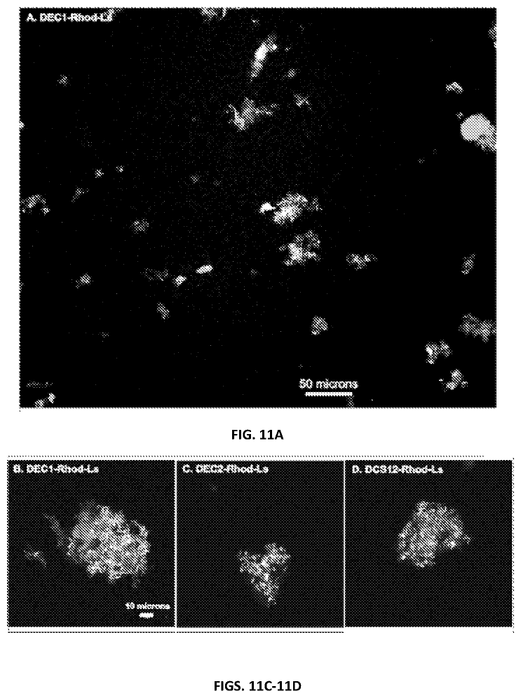

A green fluorescent protein (GFP) expressing strain of M. avium (hsp60 gfp) was grown in liquid minimal medium. The cells were fixed with formalin, washed, and blocked in buffer with BSA. The four protein coated liposomes and control uncoated Rhod-Ls were incubated with cells in blocking buffer with agitation and washed extensively. Cells and cell clusters were identified based on their green GFP fluorescence. The green and red fluorescence of randomly selected clusters were recorded using epifluorescence microscopy (FIG. 11). FIG. 11A shows a 20× magnification overview of DEC1-Rhod-L binding. M. avium cells in this preparation were found primarily in densely packed cell clusters as had been reported for M. bovis cells grown in liquid with agitation (Cantillon et al. NPJ Biofilms Microbiomes 2021:7:12. doi: 10.1038/s41522-021-00186-8). This image also suggests that DEC1-Rhod-Ls were bound to the exopolysaccharide matrices surrounding nearly every cell or cell cluster. The potential binding of the five liposome types was explored in greater detail using epifluorescence microscopy at 63× magnification. DEC1-Rhod-Ls and DEC2-Rhod-Ls bound very efficiently to a significant amount of exopolysaccharide associated with cell clusters but did not appear to be bound to the cells themselves (FIG. 11B, 11C). DCS12-Rhod-Ls bound more modestly to the exopolysaccharide associated with the cell clusters (FIG. 11D). The negative control liposomes, BSA-Rhod-Ls and uncoated Rhod-Ls were not significantly associated with cell clusters (FIG. 11E, 11F).

Quantification of Dectin-Coated Liposome Binding to M. avium

The relative binding efficiency of targeted liposomes was quantified by randomly photographing fields with 10 or more GFP M. avium cells in a cluster and their associated Rhodamine B red fluorescent liposomes at 63× magnification. The area of targeted liposome binding was quantified (FIGS. 12A and 12B) and compared to the amount of background red fluorescence recorded in images of the untargeted control Rhod-Ls. Respectively, DEC1-Rhod-Ls, DEC2-Rhod-Ls, and DCS12-Rhod-Ls bound M. avium cells 530-fold (P=5.4×10⁻⁵), 550-fold (P=0.0019), and 50-fold (P=0.025) more efficiently than untargeted Rhod-Ls (FIG. 12A). Replicate experiments with M. avium produced similar results showing highly significant binding for DEC1-Rhod-Ls and DEC2-Rhod-Ls However, red fluorescence from DCS12-Rhod-L binding was not always significantly above background in the Rhod-L sample. The level of binding of the protein coated control BSA-Rhod-Ls was statistically indistinguishable from that of Rhod-Ls. The result with BSA-Rhod-Ls inferred that a protein coating alone was not responsible for the strong binding of the C-type lectin coated liposomes.

The Glycan Specificity of Dectin-Coated Liposome Binding to M. avium

Other studies showed that Dectin-1 and Dectin-2 coated liposomes recognize their respective beta-glucan and alpha-manna cognate oligoglycans when binding pathogenic fungi (Ambati et al., 2019). To confirm that these two Dectins bound to their respective cognate ligands in M. avium's exopolysaccharide matrix, laminarin, a 6 kDa oligo-beta-glucan and yeast oligomannan, and a 133 kDa oligo-alpha-mannan were employed and their relative ability to inhibit binding was examined. These two oligosaccharides were expected to contain many. but not all, of the variously crosslinked oligoglucan and oligomannan structures found in mycobacteria's exopolysaccharide matrix. DEC1-Rhod-Ls and DEC2-Rhod-Ls were preincubated with each oligosaccharide and this mixture was used to treat M. avium cells. The area of red fluorescent liposomes surrounding randomly selected cell cluters was measured in multiple images for each treatment as shown in FIG. 12. Laminarin (L) inhibited Dectin-1 specific binding of DEC1-Rhod-Ls to M. avium 3-fold relative to yeast oligomannin (M) (FIG. 12B, PMW=0.035). Conversely, yeast oligomannan (M) significantly inhibited Dectin-2 specific DEC2-Rhod-L binding to M. avium 3.9-fold relative to laminarin (L) (FIG. 12B. PMW=0.0052). Hence. Dectin-1 and Dectin-2 each preferentially target liposome to their expected cognate ligands in M. avium's exopolysaccharide matrix and binding was not due to the non-specific affinity of either Dectin. 231 232

Dectin-Coated Liposomes Bound Efficiently to M. ulcerans' Exopolysaccharide Matrix

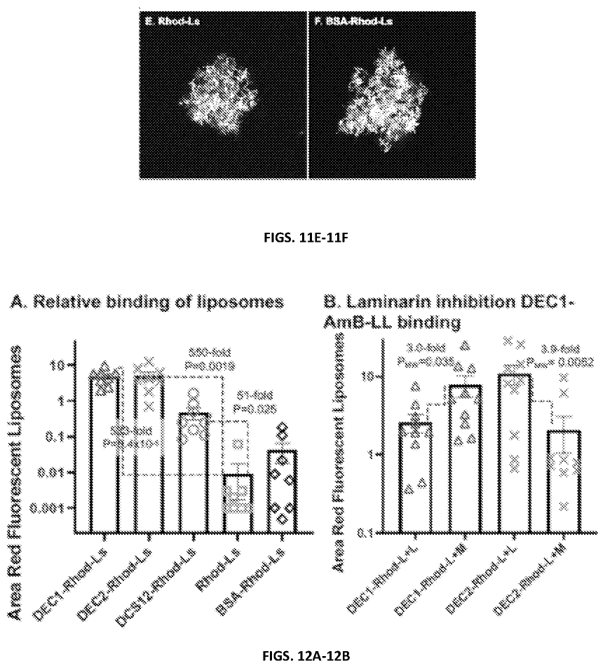

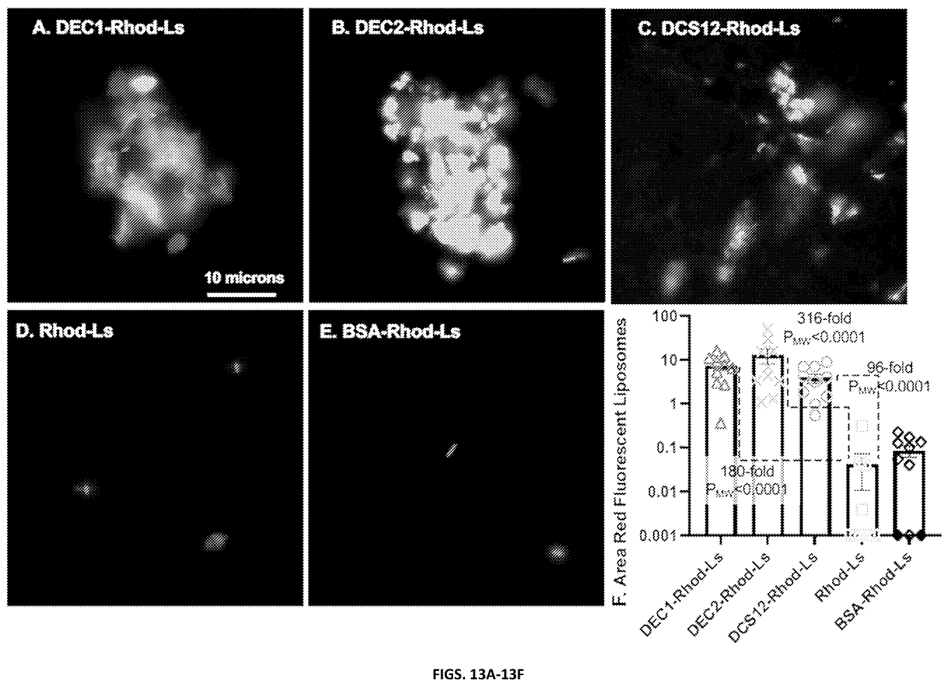

CW staining revealed that our preparations of M. ulcerans were composed mostly of singlet cells or clusters of only a few cells (FIG. 13) and very rarely contained large cell clusters. The 63×laser scanning images of blue CW staining and red fluorescent liposomes were cropped to show only ¼th of the original image (i.e., each image was magnified 4-fold relative to FIG. 11) to better present these small cell clusters and their associated Dectin-bound exopolysaccharide matrices. DEC1-Rhod-Ls and DEC2-Rhod-Ls each bound to extensive amounts of exopolysaccharide surrounding each cell or cluster as shown in FIGS. 13A and 13B, respectively, while Rhod-Ls or BSA-Rhod-Ls did not bind significantly (FIGS. 13D and 13E). respectively. DCS12-Rhod-Ls bound to material in the M. ulcerans cell preparations but was not closely associated with the cells themselves (FIG. 13C). Only rare background traces of binding were detected for Rhod-Ls and BSA-Rhod-Ls. The same method was used to quantify the area of liposome binding as in FIG. 12. Respectively, DEC1-Rhod-Ls, DEC2-Rhod-Ls, and DCS12-Rhod-Ls bound 180-fold (PMW<0.0001), 316-fold (PMW, 0.0001) and 96-fold (PMW<0.0001) more efficiently to these cell preparations than Rhod-Ls (FIG. 13F). The wide distribution of DCS12-Rhod-L binding surrounding a cell(s) is likely because they bound to exopolysaccharide that became partially dissociated during the various treatment and washing steps. A replicate experiment produced very similar qualitative and quantitative data for all three classes of targeted liposomes.

Dectin-Coated Liposomes Bound Efficiently and Quantitatively to Exopolysaccharide Associated with Pellicle-Grown M. tuberculosis

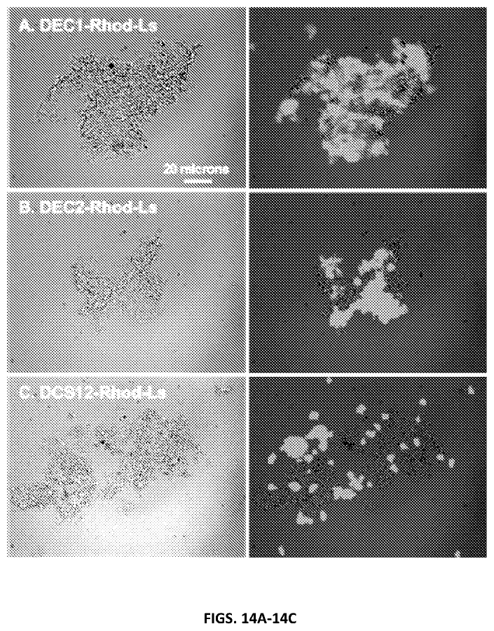

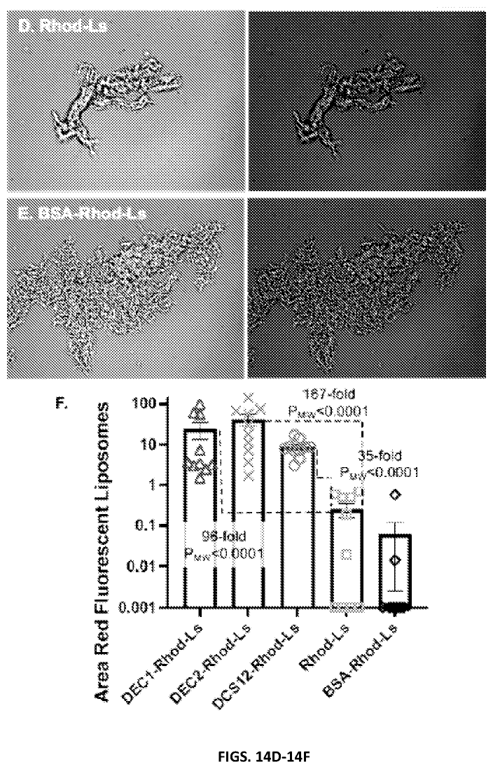

When M. tuberculosis is grown as a pellicle or biofilm it better expresses the extracellular oligoglycans and glycoproteins reflective of cells grown in an infected host. Therefore. M. tuberculosis was grown in pellicle culture. The pellicle was dispersed prior to fixation, blocking, and binding by various preparations of liposomes. M. tuberculosis could not be stained with the chemical analog of CW that was employed. Bright field microscopy identified cell clusters composed of hundreds of cells (FIG. 14). DEC1-Rhod-Ls, DEC2-Rhod-Ls, and DCS12-Rhod-L each bound extensively to exopolysaccharide that was closely associated with the cell clusters (FIG. 14A, 14B, 14C). Although DCS12-Rhod-L binding binding was dispersed in small patches (FIG. 14C) and was somewhat less extensive than the binding by the two dectin-targeted liposomes. Again, only traces of back bound binding were detected for Rhod-Ls or BSA-Rhod-Ls (FIG. 14D, 14E). Quantitative data was obtained by photographing randomly identified cell clusters in bright field and co-recording liposomal red epifluorescence. Respectively, DEC1-Rhod-Ls bound 96-fold (PMW<1×10⁴), DEC2-Rhod-Ls bound 167-fold (PMW<1×10⁻⁴) and DCS12-Rhod-Ls bound 35-fold (PMW<1×10⁻⁴) more efficiently than untargeted control Rhod-Ls (FIG. 14F). By examining the distribution of data points in each bar in this log 10 scaled scatter bar plot, it is clear the three C-type lectin coated liposomes bound efficiently and well above background levels to almost every cell cluster that was examined. A replicate experiment produced very similar results, wherein all three C-type lectin coated liposomes bound one to two orders of magnitude more efficiently than Rhod-Ls.

These studies shows that exemplary CRDs and stalk regions of Dectin-1 and Dectin-2 efficiently targeted liposomes to the exopolysaccharide matrices associated with three diverse species of pathogenic mycobacteria. M. avium, M. ulcerans, and M. tuberculosis. The CRD and two neck repeats of the DC-SIGN, isoform DCS12, efficiently targeted liposomes to the exopolysaccharides produced by M. ulcerans and M. tuberculosis.

### The Ligand Specificities of Dendritic Cell Dectin-1, Dectin-2, and DC-SIGN and Potential Binding to Mycobacteria

Dendritic cell Dectin-1, Dectin-2, and DC-SIGN are among several dendritic cell C-type lectin receptors that recognize oligosaccharides and glycoproteins produced by pathogenic mycobacteria and signal host immune responses. Binding of Dectin-1. Dectin-2 and DC-SIGN to mycobacteria had been studied in dendritic cells, but the binding of the purified proteins themselves had not been demonstrated. It was therefore possible that these C-type lectins were only accessary proteins collaborating with other receptors in dendritic cell recognition of and binding to mycobacteria, but not sufficient for the efficient binding we sought. However, the data presented herein demonstrate clearly that DectiSomes targeted by Dectin-1 or Dectin-2 or DC-SIGN each bind tightly and efficiently to extracellular oligosaccharides produced by mycobacteria. No clear or significant binding to the cell walls of any of these diverse Mycobacteria spp. was observed, yet the cell walls of that pathogenic mycobacteria that have been examined include cognate ligands for these C-type lectins. It is possible that the cell wall epitopes were chemically masked by various endogenous chemical crosslinking of oligoglycan and oligolipoglycan residues or that tight crosslinking of the cell walls spatially restricted access to 100 nanometer liposomes. It is also possible that the formalin fixation of the samples damaged cell wall epitopes and cell wall binding might have been observed with live cells.

The Advantages Derived from the Design of Targeted Liposomes

The liposomal design and composition used herein, shown in FIG. 10B and Table 3, respectively, afford a few special advantages for examining binding and localization of Dectin-1-. Dectin-2-, and DC-SIGN-coated nanoparticles to mycobacteria's cognate ligands and the targeted delivery of anti-mycobacterial drug loaded liposomes. First, each 100-nanometer diameter liposome contains a few thousand molecules of rhodamine B producing a very strong red fluorescence signal once bound. Protein reagents such as antibodies and secondary fluorescent antibodies cannot produce such strong fluorescent signals. Secondly, each targeted liposome contains more than a thousand molecules of a C-type lectin CRD (Table 3). These proteins function best when they can float together in a membrane and form homomultimers that bind their cognate oligoglyans. Furthermore, when multiple homomultimers on one liposome bind to their mycobacterial cognate ligands, this provides avidity to their binding.

Avidity may be particularly important to binding oligoglycan ligands with low affinity constants as it is for pentameric IgM antibodies binding to antigens early in an immune response, when antibody affinity is weak. Hence, when each receptor protein coated liposome produces more durable binding to mycobacteria than may be provided by any fluorescently tagged C-type lectin protein alone or an antibody alone. Third. C-type lectin protein CRDS themselves are very hydrophobic and insoluble and are generally employed as fusion proteins that are difficult to produce and purify. Dectin-1. Dectin-2 and DC-SIGN isoform CDRs are relatively stable when presented on the surface of a liposome. Fourth, the closest analog to the targeted liposomes provided herein (for example. DectiSomes), immunoliposomes, have been used to successfully to target toxic anti-cancer drugs. However, monoclonal antibody reagents are orders of magnitude more expensive to produce than C-type lectin reagents. The much lower cost of C-type lectin targeted antibacterial therapeutics will aid the development of anti-mycobacterial drugs that can be used to treat millions of individuals, for example, those in the poorest countries most affected by TB.

### Overcoming the Current Limitations of Antibacterial Drug Development

For the past several decades antibacterial drug development including that for TB has progressed primarily through the systematic stepwise improvement in the chemical design of existing classes of antibacterial drugs, resulting in incremental improvements in drug efficacy, usually for one phylogenic class of bacteria. Fewer new drugs are clinical successes. The lack of success results in part from the fact that there are too many new drug variants for physicians to choose from and, because most new drugs are highly specialized and only provide modest advantages against certain pathogens, confusing a physician's choices. The robust evidence provided herein shows that that C-type lectin pathogen receptors can target nanoparticles to three species of pathogenic mycobacteria. The targeted nanoparticles described herein have potential as a pan-bacterial pathogen drug delivery system to improve antibacterial drug efficacy (FIG. 10B).

Efficiency and Kinetics of DEC1-RIF-LLS, DEC2-RIF-LL and DCS12-RIF-LL Binding to M. tuberculosis, M. avium, and M. ulcerans Grown In Vitro Under Diverse Conditions.

The binding of targeted DEC1-rifampin (RIF)-LLs. DEC2-RIF-LLs and DCS12-RIF-LLs to M. tuberculosis, M. avium, and M. ulcerans will be compared and quantified relative to untargeted RIF-LLs and BSA-RIF-LLs. In vitro grown M. tuberculosis strain Erdman (ATCC35801) and M. ulcerans (ATCC19423) will be visualized and quantified using calcofluor white (CW) staining. CW stains mycobacterial cells, likely recognizing complex glucopyranose polymers. It was found that CW produced a 10-fold stronger fluorescent signal than the GFP in M. avium (hsp60 gfp), simplifying cell detection over GFP.

In addition to the binding studies, the specificity of DEC1-RIF-LL and DEC2-RIF-LL binding to their cognate ligands will be confirmed by showing that their binding is specifically inhibited by the oligoglucan laminarin and by yeast oligomannan, respectively. The binding kinetics of DEC1-RIF-LLs. DEC2-RIF-LLs and DCS12-RIF-LLs to all three Mycobacterium spp. will also be determined. Liposome binding to cells grown in Oleic acid, albumin, dextrose and catalase (OADC) supplemented Middlebrook 7H9 liquid medium with agitation, and to cells grown as a planktonic biofilm in glass culture bottles in modified Sauton's medium without agitation, will also be examined. Planktonic cell masses will be fixed and agitated before liposome labeling and viewing by microscopy.

Mycobacterium spp. express diverse oligo-glycans and lipoglycans in their walls and exopolysaccharide matrices. It is expected that all three classes of DectiSomes (DEC1-RIF-LLs. DEC2-RIF-LLs and DCS12-RIF-LLs) will bind significantly to M. tuberculosis, M. avium, and M. ulcerans, but their relative efficiencies of binding may differ.

Effective Dose (ED) of RIF Delivered by DEC1-RIF-LLs and DEC2-RIF-LLs for 95% Inhibition and Killing (ED₉₅) of M. tuberculosis, M. avium and M. ulcerans

RIF's activity is considered bactericidal but may initially be bacteriostatic. Because cytotoxicity assays require at least a 3- to 5-day growth period during drug treatment, inhibition cannot easily be distinguished from killing, hence, these experiments are referred to as inhibition and killing assays. Two quantitative assays will be used to assess the ability of targeted liposomes to inhibit and/or kill Mycobacterium spp. as compared to untargeted liposomes and free RIF. AlamarBlue® (Promega) cell viability assays will be used to quantify the growth inhibition and/or killing as is described previously for M. tuberculosis and the other Mycobacterium spp. (Franzblau, et al. “Rapid, low-technology MIC determination with clinical Mycobacterium tuberculosis isolates by using the microplate Alamar Blue assay,” J Clin Microbiol 36, 362-366 (1998): Carroll, et al. “Optimization of a rapid viability assay for Mycobacterium avium subsp. paratuberculosis by using alamarBlue. Applied and environmental microbiology 75, 7870-7872 (2009): Amin, et al. “Rapid screening of inhibitors of Mycobacterium tuberculosis growth using tetrazolium salts,” Methods in molecular biology (Clifton, N.J.) 465, 187-201 (2009). Several classes of NADPH-dependent reductases electrochemically reduce the active resazurin component of AlamarBlue® reagent to fluorescent resorufin. Resorufin's fluorescence emission is measured at 590 nanometers. These reductase activities require an intact electron transport chain, which is not active in dead or metabolically inactive cells. The closely related CellTiter Blue® resazurin redox assay has been used extensively. AlamarBlue is reported to be better for assays conducted over longer time periods than CellTiter Blue. The AlamarBlue assay generates easily quantifiable data in a 96 well microtiter plate format and it has a wider dynamic range than colorimetric metabolic assays or cell density assays.

Cell density will be assayed by spectrophotometry in a microtiter plate format. This assay will provide strong confirmatory data, making results more robust. In a typical cytotoxicity assay, cells will be grown to early log phase in microtiter plates in ADC modified Middlebrook 7H9 medium¹, then incubated with various concentrations of drugs in the growth medium for 3 to 4 more days, and assayed first for cell density and secondly for residual reductase activity by incubating overnight with AlamarBlue reagent. Using CellTiter Blue microtiter plate assays, six replicate wells assayed for each effective treatment condition typically give highly statistically significant data. Optionally, another assay can be performed, where the cells are treated in liquid, cell dilutions are plated on agar, and assay colony forming units (CFUs) are determined. (Satti et al., “A. Rapid direct testing of susceptibility of Mycobacterium tuberculosis to isoniazid and rifampin on nutrient and blood agar in resource-starved settings,” J Clin Microbiol 50, 1659-1662 (2012)).

In previously published studies, the ED₉₅ values for free RIF against various RIF sensitive strains was typically below 0.5 μg/mL and above 2 μg/mL for resistant strains. The WHO has recently lowered the cutoff for distinguishing sensitive and drug resistant strains from 1.0 to 0.5 ug/mL RIF. It is expected that both DEC1-RIF-LLs and DEC2-RIF-LLs can reduce the ED₉₅ of RIF for these mycobacteria, for example, by an order(s) of magnitude relative to RIF-LLs or free RIF, with AlamarBlue reagent showing larger effects than cell density assays. It is possible that ED₉₅ values of 0.02 to 0.05 ug/ml for DEC1-RIF-LLs and DEC2-RIF-LLs, one or two orders of magnitude lower than for RIF-LLs or free RIF (Franzblau, (1998): Carroll, (2009)), can be obtained.

Mouse M. tuberculosis Aerosol Infection Model

A mouse M. tuberculosis aerosol infection model will be used to demonstrate that DEC1-RIF-LLs and DEC2-RIF-LLs delivered by oral aspiration, can lower the effective dose of RIF, to reduce mycobacterial burden and improve lung pathophysiology, relative to untargeted RIF.

All animal experiments will be performed following protocols approved by the Institutional Animal Care and Use Committee at the University of Georgia. Gamma interferon disrupted INFγ KO mice have been used previously for TB drug susceptibility testing studies, and thus will be used (Lenaerts, et al. “Rapid in vivo screening of experimental drugs for tuberculosis using gamma interferon gene-disrupted mice. Antimicrob Agents Chemother 47, 783-785 (2003)).

Three mice per group (4 groups: 12 mice) will be treated with either 0.25 mg/kg or 0.5 mg/kg of DEC1-RIF-LLS, DEC2-RIF-LL, RIF-LL or free RIF. At 0, 30-, 60-, 120-, and 180-minutes post treatment, the mice will be bled, sera prepared and stored at −80C. The bioavailability of RIF will be tested by microdilution assay. Briefly, sera will be diluted with 7H9 broth and inoculated with very low dose of M. tuberculosis Erdman bacilli in a 96-well plate. Briefly, M. tuberculosis Erdman will be cultured to an absorbance of 0).2, dilute 1:25 in 7H9 broth medium and add 100 μL per well along with 100 μL of serum in duplicate plates. The plates will be kept at 37° C. and absorbance at 600 nm will be measured every day for 20 days for one plate. The duplicate plate will be kept at 37° C. for 3 days. After incubation, 25 μl Alamar blue diluted 1:1 with 10% Tween 80 will be added per well and kept at 37 C for 16 hours. Color change from blue to pink will be recorded.

Mice will be challenged with 50-100 CFU of M. tuberculosis strain Erdman (ATCC35801), using a Madison aerosol chamber. Four infected mice will be sacrificed 24 hours post-infection to confirm the lung infection dose of M. tuberculosis. All mice will be monitored daily for lethargy, weight loss, hunched back or ruffled hair. At day 18, 6 mice will be euthanized to determine bacterial lung load and remaining 6 mice per group (6 groups: 36 mice) will be given a maximum of 100 μl oropharyngeal treatments. The 6 groups include: DEC1-RIF-LLS, DEC2-RIF-LLS, RIF-LLs, free RIF, 25 mg/kg isoniazid, and empty liposomes. The treatments will be given daily up to day 28 post infection. Optionally, the experiments will use a dose of RIF where RIF-LLs and free RIF have a small and barely significant effects, so the benefits of DectiSomes can be determined. Dosage will start with 0.25 to 0.5 mg/kg doses of RIF, significantly below the ˜10 mg/kg/day doses of RIF-LLs and RIF reported to control murine infections.

At day 30 post infection, all the mice from each group will be euthanized and portions of lungs, livers, and spleens will be harvested and placed in PBS with 0.05% Tween 80 and homogenized. Serially diluted homogenates will be plated onto 7H11gtADC agar plates and incubated at 37° C. for three weeks prior to CFU assessments. Additional portions of the lungs will be fixed in 10% neutral-buffered formalin and processed. Histopathology will be scored by a pathologist who will be blinded to the identity of the individual experimental samples. Sections will be assessed subjectively for percentage of the lung section affected by the presence of granulomas and then assessed quantitatively for the number of granulomas per section.

0.25 mg/kg free RIF or RIF-LLs are expected to have little impact on these phenotypes, but targeted liposomes will reduce M. tuberculosis organ CFUs and lung granuloma size. DEC1-RIF-LLs and DEC2-RIF-LLs could, in some instances, lower the ED₉₅ at least 5- or 10-fold. It is understood that doses of RIF (e.g., 0.25 mg/kg) and the route of administration (e.g., intranasal or intraperitoneal) can be adjusted. Additional mice will be employed if the dose of RIF needs to be increased.

### Examples Iv

### Dc-SIGN Nanoparticles and Inhibition of Parasitic Infection

Toxoplasmosis is a costly and deadly disease caused by the apicomplexan parasite, Toxoplasma gondii. Forty million people in the U.S. (i.e., 11% of our population) and perhaps 1/3 of the global population are infected with various strains of T. gondii. Humans are most often infected with T. gondii from ingestion of tissue cysts or tachyzoites in contaminated food but may inhale oocysts from feline feces when cleaning litter boxes. Once ingested, cysts and oocytes release tachyzoites, which disseminate throughout the host, burrow into diverse host tissues and cause acute disease. Bradyzoites develop from tachyzoites and form new tissue cysts in host tissues Enclosed in their bilipid membrane, tissue cysts are coated by a 800 nm thick wall that protects them from host immunity and drugs. Therefore, it could be beneficial to target drugs to the cyst wall and tachyzoites with pathogen receptor proteins, for example, with DC-SIGN or a fragment thereof, targeting tachyzoites.

There are numerous models of T. gondii available (Szabo et al., “Toxoplasma gondii: One Organism, Multiple Models. Trends Parasitol 33, 113-127 (2017)). DC-SIGN recognizes, among other ligands, a wide variety of alpha1-6 or alpha1-3 oligomannose-type N-glycans (van Liempt, et al., “Specificity of DC-SIGN for mannose- and fucose-containing glycans. FEBS Letters 580, 6123-6131 (2006)). Therefore, the ability of DC-SIGN to bind to parasite oligosaccharides can be used to target anti-parasitic drugs to T. gondii cells. Several drugs have been used to treat toxoplasmosis, with varying, and often limited effects. Azithromycin (AZI) is modestly effective against toxoplasmosis in the clinic, but most often used in conjunction with other drugs such as pyrimethamine. At effective doses AZI (250 to 900 mg/day per patient) can cause abdominal pain, diarrhea, nausea, vomiting and hearing problems, and may also cause liver disease. Using the DC-SIGN targeted nanoparticle strategy described herein, it could be possible to reduce the effective dose of AZI to treat Toxoplasmosis.

DCSIGN12 Coated Liposomes DCSIGN12-Ls Bind to the Membrane of T. gondii Tachyzoites in Infected Human Fibroblasts

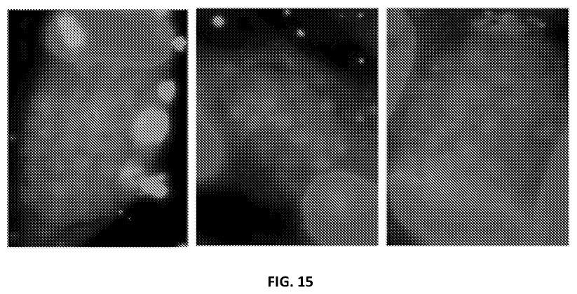

Formalin fixed samples of human foreskin fibroblasts (HFFs) infected with T. gondii at a stage PI, when both tachyzoites and tissue cysts are present were obtained. These cells were stained with rhodamine B tagged (red fluorescence, Ex553/Em627) labeled DCSIGN12-Ls, DEC1-Ls, DEC2-Ls, BSA-Ls, and uncoated liposomes. Cells were co-stained with FITC conjugated Dolichos Biflorus Agglutinin (DBA-FITC) (blue fluorescence, Ex495/Em515) tag N-acetylgalactosamine in the tissue cyst wall and DAPI for protist and host cell nuclear DNA. FIG. 15 shows DAPI blue fluorescent staining of small T. gondii cell nuclei and large human cell nuclei. DCSIGN-coated liposomes (i.e., Rhodamine conjugated DCSIGN12-Rhod liposomes) bound efficiently to tachyzoites of T. gondii strain ME49 growing in infected cultures of human foreskin fibroblasts (HFFs). Dectin-1 coated liposomes, DEC1-AmB-LLs, did not bind to T. gondii.

### AZI Loaded Liposomes

AZI Loaded Liposomes, AZI-LLs, can be prepared by remotely loading 10 moles percent AZI into commercial 100 nm liposomes (FormuMax, DSPC: CHOL: mPEG2000-DSPE, 53:47:5) using a method similar to that which was employed to load AmB into AmB-LLs (Ambati et al., 2019b).

DCSIGN12-AZI-LLs Effect on the Number of T. gondii Tachyzoites and Tissue Cysts Produced In Infected HFF Culture Cells Relative to Untargeted AZI

In vitro experiments will be performed using Human Foreskin Fibroblasts, HFFs, immortalized with human telomerase reverse transcriptase (hTERTs: ATCC CRL-4001). T. gondii strain ME49-cLuc-GFP, which offers the advantage of GFP monitoring of both tachyzoites and bradyzoites at 20× magnification, will be used. It is expected that DCSIGN12-AZI-LLs will reduce the number of tachyzoites, and tissue cysts produced by in vitro grown T. gondii relative to untargeted AZI-LLs or free AZI. Monolayers of HFFs will be grown and infected using an MOI of 0.5 tachyzoites per HFF. Two to four hours later, cells will be treated with DCSIGN12-AZI-LLS, DEC1-AZI-LLS, DEC2-AZI-LL, AZI-LLs, and free AZI, wherein the latter three serve as negative untargeted AZI controls and with liposome dilution buffer. The reduction in the number of tachyzoites and tissue cysts produced by DCSIGN12-AZI-LLS relative to the untargeted drug controls will be monitored and quantified from 1 to 8 days (D1 to D8) post infection (PI) by several methods, as described below.

Microscopy: For this assay, HFF cells are grown and infected on 18×18 mm coverslips in 12 well microtiter plated. The number of tachyzoites and tissue cysts present in the cells following liposome treatment will be counted on D1 and D2 PI, using epifluorescence microscopy after staining with DCSIGN12-AZI-LLS, DCSIGN12-Rhod for tachyzoites and DBA for tissue cysts, and DAPI for DNA. Ten to twenty fluorescent images will be taken for each treatment and the numbers of parasites counted will be normalized to the numbers of host cell nuclei per image.

Plaque Forming Units (PFUs): For this assay and the next cytometric assay the cells are grown and infected in 12 well microtiter plates and given drug treatments. On D8, the number of viable parasites will be performed by plating a volume equivalent to ˜200 parasites for the buffer control and more for the drug treated samples on a lawn of early stage HFFs and allowing them to grown for 8 days (Stasic et al., 2019). Intact HFF cells are stained with crystal violet, which highlights the clear unstained plaques, where cells are lysed. The reduced number of Plaque Forming Units (PFUs) for cells treated with DCSIGN12-AZI-LLs relative to control liposomes will be estimated. It is noted that, only tachyzoites are capable of HFF host cell attachment and infection and tissue cysts are not, so plaque assays measure the number of tachyzoites released at the end of the lytic cycle.

Cytometry: Tachyzoites released following host cell lysis at D8 PI will be washed and filtered free of cellular debris, formalin fixed stained for their dsDNA content with DAPI (Ex254/Em520) (Hu et al., 2004). Tachyzoites are haploid and those released by natural cell lysis will primarily be IN cells with some small fraction of 2N cells. The number of cells following drug treatment will be quantified by cytometry. The DAPI blue, fluorescent channel of the cytometer will be gated to detect cells containing small IN and 2N T. gondii nuclei, and the green channel gated to detect GFP stained cells and the forward FSC and side scatter SSC channel for T. gondii sized cells. LDH release assays can also be performed.

In Vivo T. gondii Studies

CD-1 (CDI) outbred Swiss mice will be used (Muller et al., “Development of a murine vertical transmission model for Toxoplasma gondii oocyst infection and studies on the efficacy of bumped kinase inhibitor (BKI)-1294 and the naphthoquinone buparvaquone against congenital toxoplasmosis,” J Antimicrob Chemother 72, 2334-2341 (2017)). Type I strain of T. gondii will be used, as immunocompetent CDI mice are easily infected with Type I strains of T. gondii Treatment regimens. An exemplary regimen for infection, drug treatment, and analysis of phenotypes on mice with toxoplasmosis is as follows. On Day ((DO) 7- to 9-week-old (27 to 30 g) mice will be infected by intraperitoneal infection or in separate experiments by by oropharyngeal delivery of with 10,000 T. gondii tachyzoites. Mice will be given sulfadiazine in their drinking water (400 mg/L) from day 1 to day 30 to reduce the proliferation of tachyzoites, to improve mouse survival, and to encourage the development of encysted bradyzoites. Every day, from D1 to D10, they will be given an intravenous treatment with AZI either suspended in PBS (Free AZI) or packaged in targeted liposomes, DCSIGN12-AZI-LL, or untargeted liposomes, AZI-LLs, or PBS Buffer Control. In previous studies, 200 mg/kg per day Free AZI (˜6 mg/mouse) for 10 days gave 100% mouse survival. Dosage will be started at a 10 to 20-fold lower dose of AZI (i.e., 10 to 20 mg/kg). This will provide information regarding lower the effective dose of drug. There will be 12 mice in each treatment group in the following experiments.

It is expected that rhodamine tagged DCSIGN12-AZI-LLs will be concentrated around GFP fluorescing T. gondii cells. Hand sections of infected organs will be examined, top down, by epifluorescence. The burden of T. gondii cells remaining the lungs and brain will be assayed between D5 and D10, with the exact day being determined based upon the day when control infected animals become moribund. The viability of both tachyzoites and tissue cyst bradyzoites in homogenized organ samples will be assayed with the plaque assay (Wang and Sibley, 2020) and by quantitative Real Time qPCR of the rDNA associated intergenic transcribed spacer ITS1 The dCT method (Livak and Schmittgen, “Analysis of relative gene expression data using real-time quantitative PCR and the 2 (-Delta Delta C (T)) Method,” Methods 25, 402-408) (2001)) will be used to normalize analysis of qPCR data and obtain a Relative Quantity (RQ) from different experiments. Plaque forming Units (PFU) and RQ data from drug treated mice will be compared to control infected animals.

Because the survival in bradyzoites in tissue cysts after drug therapy is considered a major cause of drug treatment failures, both tachyzoites and tissue cysts in fixed brain and lung tissue sections will be monitored. These organs will be chopped into ˜1 mm sq pieces and multiple tissue pieces fixed and embedded for paraffin sectioning and stained.

Survival time will be monitored to D30. On D30, the average survival time and mortality rate calculated as a percent of mice surviving will be calculated for each treatment group.

Relative to untargeted drugs, it is expected that oligoglycan targeted DCSIGN12-AZI-LLs will reduce tachyzoites and tissue cyst burden in the lungs of the mice, improve the rates of mouse survival, and/or lower the mg/kg effective dose of AZI.

SEQUENCES
SEQ ID NO: 1 Carbohydrate Recognition Domain (CRD) of DC-SIGN
CHPCPWEWTFFQGNCYFMSNSQRNWHDSITACKEVGAQLVVIKSAEEQNFLQLQSS
RSNRFTWMGLSDLNQEGTWQWVDGSPLLPSFKQYWNRGEPNNVGEEDCAEFSGNG
WNDDKCNLAKFWICKKSAASCSRDEEQFLSPAPATPNPPPA
SEQ ID NO: 2 (NR1) of DC-SIGN
QSRQDAIYQNLTQLKAAVGEL
SEQ ID NO: 3 (NR2) of DC-SIGN
SEKSKLQEIYQELTQLKAAVGEL
SEQ ID NO: 4 (NR3) of DC-SIGN
PEKSKLQEIYQELTRLKAAVGEL
SEQ ID NO: 5 (NR4) of DC-SIGN
PEKSKLQEIYQELTWLKAAVGEL
SEQ ID NO: 6 (NR5) of DC-SIGN
PEKSKMQEIYQELTRLKAAVGEL
SEQ ID NO: 7 (NR6) of DC-SIGN
PEKSKQQEIYQELTRLKAAVGEL
SEQ ID NO: 8 (NR7) of DC-SIGN
PEKSKQQEIYQELTRLKAAVGEL
SEQ ID NO: 9 (NR8) of DC-SIGN
PEKSKQQEIYQELTQLKAAVERL
SEQ ID NO: 10 (full-length DCS78)

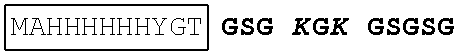

GELPEKSKQQEIYQELTRLKAAVGELPEKSKQQEIYQELTQLKAAVERLCHPCPWEW
TFFQGNCYFMSNSQRNWHDSITACKEVGAQLVVIKSAEEQNFLQLQSSRSNRFTWM
GLSDLNQEGTWQWVDGSPLLPSFKQYWNRGEPNNVGEEDCAEFSGNGWNDDKCN
LAKFWICKKSAASCSRDEEQFLSPAPATPNPPPA
SEQ ID NO: 11 (nucleic acid encoding DCS78)
GGTACCATGGCTCACCATCACCACCACCATTATGGAACTGGTTCTGGCAAGGGCA
AGGGCAGCGGCAGCGGTGGAGAACTACCCGAGAAGTCAAAACAGCAAGAGATT
TACCAGGAGTTGACTCGTCTGAAGGCGGCGGTGGGCGAACTTCCGGAAAAATCG
AAACAGCAGGAGATCTACCAAGAGTTGACGCAGTTGAAGGCGGCGGTTGAACGT
CTGTGTCATCCGTGTCCGTGGGAATGGACCTTCTTCCAGGGCAACTGCTATTTCA
TGTCTAACAGCCAGAGAAATTGGCACGACAGCATTACCGCATGTAAAGAAGTTG
GTGCACAGCTGGTGGTGATCAAATCTGCGGAGGAACAAAACTTTCTCCAACTGC
AATCCAGCCGTAGCAATCGTTTTACCTGGATGGGTCTGAGCGACCTGAATCAGGA
GGGCACCTGGCAGTGGGTTGACGGCTCGCCGCTGCTGCCATCATTTAAACAATAT
TGGAACCGCGGTGAACCGAACAACGTCGGTGAGGAAGATTGCGCCGAGTTCAGC
GGTAACGGCTGGAACGATGACAAGTGCAATCTGGCAAAGTTCTGGATCTGCAAG
AAATCCGCGGCCAGCTGCAGCCGCGATGAAGAGCAATTTTTATCCCCGGCTCCG
GCGACCCCGAATCCGCCTCCGGCTTAATTAA
SEQ ID NO: 12 (full-length DCS12)

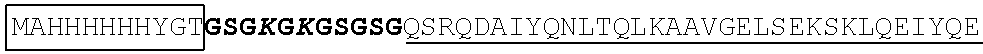

LTQLKAAVERLCHPCPWEWTFFQGNCYFMSNSQRNWHDSITACKEVGAQLVVIKSA
EEQNFLQLQSSRSNRFTWMGLSDLNQEGTWQWVDGSPLLPSFKQYWNRGEPNNVG
EEDCAEFSGNGWNDDKCNLAKFWICKKSAASCSRDEEQFLSPAPATPNPPPA
SEQ ID NO: 13 (nucleic acid encoding DCS12)
GGTACCATGGCTCACCATCACCACCACCATTATGGAACTGGTTCTGGCAAGGGTA
AGGGTAGCGGCTCTGGCCAGAGCAGACAAGACGCAATTTACCAGAACCTGACCC
AGCTTAAGGCGGCGGTTGGCGAGCTGTCCGAAAAAAGCAAACTGCAAGAGATCT
ACCAAGAGTTGACGCAGTTGAAGGCTGCTGTGGAACGTCTGTGCCATCCGTGTCC
GTGGGAATGGACCTTTTTCCAGGGTAACTGCTATTTCATGAGCAACAGCCAGCGT
AATTGGCACGACTCGATCACCGCGTGCAAAGAAGTTGGTGCACAGCTGGTCGTG
ATCAAAAGCGCGGAGGAACAAAACTTTTTGCAACTCCAATCAAGTCGCTCCAAT
CGTTTCACCTGGATGGGTCTGTCGGATCTGAATCAGGAGGGCACCTGGCAATGG
GTTGATGGCAGCCCGCTGCTGCCGAGCTTTAAACAGTATTGGAACCGCGGTGAG
CCGAATAACGTGGGCGAAGAAGATTGTGCGGAGTTCAGCGGTAATGGTTGGAAC
GACGACAAGTGCAACCTGGCGAAATTCTGGATTTGTAAAAAGTCTGCAGCCTCCT
GCAGCCGTGATGAAGAGCAGTTTTTGTCCCCGGCGCCAGCTACGCCGAACCCGC
CGCCTGCTTAATTAA
SEQ ID NO: 14 (DCS12345678)

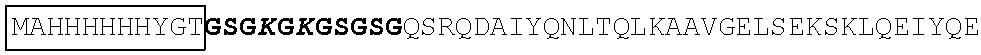

LTQLKAAVGELPEKSKLQEIYQELTRLKAAVGELPEKSKLQEIYQELTWLKAAVGEL
PEKSKMQEIYQELTRLKAAVGELPEKSKQQEIYQELTRLKAAVGELPEKSKQQEIYQ
ELTRLKAAVGELPEKSKQQEIYQELTQLKAAVERLCHPCPWEWTFFQGNCYFMSNS
QRNWHDSITACKEVGAQLVVIKSAEEQNFLQLQSSRSNRFTWMGLSDLNQEGTWQ
WVDGSPLLPSFKQYWNRGEPNNVGEEDCAEFSGNGWNDDKCNLAKFWICKKSAAS
CSRDEEQFLSPAPATPNPPPA
SEQ ID NO: 15 (na encoding DCS12345678)
ATGGCTCACCATCACCACCACCATTATGGAACCGGTAGCGGCAAGGGTAAAGGC
TCTGGTTCTGGTCAGAGCCGCCAGGATGCGATTTATCAAAACCTGACCCAACTGA
AGGCTGCTGTGGGTGAACTTTCAGAAAAGAGCAAGTTACAAGAAATTTACCAAG
AGCTCACGCAGCTGAAAGCGGCCGTTGGCGAGTTGCCGGAGAAGTCGAAGCTGC
AAGAGATTTACCAAGAACTTACCCGTTTGAAGGCTGCGGTAGGGGAGCTGCCGG
AAAAATCCAAACTGCAAGAAATCTACCAGGAGCTCACGTGGCTGAAGGCTGCGG
TGGGCGAGTTGCCAGAAAAGTCCAAAATGCAGGAGATCTACCAAGAGCTGACGC
GTTTAAAGGCGGCGGTCGGCGAGCTGCCGGAAAAATCTAAGCAACAGGAGATCT
ACCAGGAGTTGACTCGCTTAAAGGCGGCTGTGGGCGAGTTGCCGGAAAAGTCCA
AGCAACAAGAGATCTACCAAGAGTTAACACGTTTGAAAGCGGCGGTCGGTGAGT
TGCCAGAAAAGTCGAAACAGCAAGAGATCTATCAGGAACTGACCCAGCTCAAAG
CCGCCGTGGAAAGACTGTGCCATCCGTGTCCGTGGGAATGGACCTTTTTCCAAGG
TAACTGCTATTTCATGAGCAACAGCCAGCGTAATTGGCACGACAGCATTACCGCA
TGTAAAGAAGTTGGCGCACAGCTGGTTGTGATTAAATCTGCAGAAGAACAAAAT
TTTCTGCAGCTGCAGAGCAGCCGCTCGAACCGTTTCACCTGGATGGGTCTGTCCG
ACCTGAATCAGGAGGGCACCTGGCAGTGGGTTGATGGTTCGCCGCTGCTGCCGA
GCTTTAAACAGTATTGGAATCGCGGTGAGCCGAACAACGTTGGCGAGGAAGATT
GTGCGGAATTTAGCGGCAACGGTTGGAACGACGACAAATGCAATCTGGCAAAAT
TCTGGATCTGCAAGAAATCCGCAGCGAGTTGCAGCCGTGATGAAGAGCAGTTCC
TGAGCCCGGCGCCGGCGACCCCGAACCCGCCTCCGGCT
SEQ ID NO: 16
MSDSKEPRLQQLGLLEEEQLRGLGFRQTRGYKSLAGCLGHGPLVLQLLSFTLLAGLL
VQVSKVPSSISQEQSRQDAIYQNLTQLKAAVGELSEKSKLQEIYQELTQLKAAVGELP
EKSKLQEIYQELTRLKAAVGELPEKSKLQEIYQELTWLKAAVGELPEKSKMQEIYQE
LTRLKAAVGELPEKSKQQEIYQELTRLKAAVGELPEKSKQQEIYQELTRLKAAVGEL
PEKSKQQEIYQELTQLKAAVERLCHPCPWEWTFFQGNCYFMSNSQRNWHDSITACK
EVGAQLVVIKSAEEQNFLQLQSSRSNRFTWMGLSDLNQEGTWQWVDGSPLLPSFKQ
YWNRGEPNNVGEEDCAEFSGNGWNDDKCNLAKFWICKKSAASCSRDEEQFLSPAP
ATPNPPPA
SEQ ID NO: 17
MSDSKEPRLQQLGLLEEEQLRGLGFRQTRGYKSLAGCLGHGPLVLQLLSFTLLAGLL
VQVSKVPSSISQE
SEQ ID NO: 20
IWRSNSGSNTLENGYFLSRNKENHSQPTQSSLEDSVTPTKAVKTTGVLSSPCPPNWIIYEKSCY
LFSMSLNSWDGSKRQCWQLGSNLLKIDSSNELGFIVKQVSSQPDNSFWIGLSRPQTEVPWLW
EDGSTFSSNLFQIRTTATQENPSPNCVWIHVSVIYDQLCSVPSYSICEKKFSM
SEQ ID NO: 21
TYHFTYGETGKRLSELHSYHSSLTCFSEGTKVPAWGCCPASWKSFGSSCYFISSEEKVWSKSE
QNCVEMGAHLVVENTEAEQNFIVQQLNESFSYFLGLSDPQGNNNWQWIDKTPYEKNVRFW
HLGEPNHSAEQCASIVFWKPTGWGWNDVICETRRNSICEMNKIYL
SEQ ID NO: 22
HNFSRCKRGTGVHKLEHHAKLKCIKEKSELKSAEGSTWNCCPIDWRAFQSNCYFPLTDNKT
WAESERNCSGMGAHLMTISTEAEQNFIIQFLDRRLSYFLGLRDENAKGQWRWVDQTPFNPR
RVFWHKNEPDNSQGENCVVLVYNQDKWAWNDVPCNFEASRICKIPGTTLN
SEQ ID NO: 23
FWRHNSGRNPEEKDSFLSRNKENHKPTESSLDEKVAPSKASQTTGGFSQSCLPNWIMHGKSC
YLFSFSGNSWYGSKRHCSQLGAHLLKIDNSKEFEFIESQTSSHRINAFWIGLSRNQSEGPWFW
EDGSAFFPNSFQVRNAVPQESLLHNCVWIHGSEVYNQICNTSSYSICEKEL
SEQ ID NO: 24
IMDQPSRRLYELHTYHSSLTCFSEGTMVSEKMWGCCPNHWKSFGSSCYLISTKENFWSTSEQ
NCVQMGAHLVVINTEAEQNFITQQLNESLSYFLGLSDPQGNGKWQWIDDTPFSQNVRFWHP
HEPNLPEERCVSIVYWNPSKWGWNDVFCDSKHNSICEMKKIYL
SEQ ID NO: 25
HNFSRCKRGTGVHKLEHHAKLKCIKEKSELKSAEGSTWNCCPIDWRAFQSNCYFPLT
DNKTWAESERNCSGMGAHLMTISTEAEQNFIIQFLDRRLSYFLGLRDENAKGQWRW
VDQTPFNPRRVFWHKNEPDNSQGENCVVLVYNQDKWAWNDVPCNFEASRICKIPG
TTLN
SEQ ID NO: 40
GELPEKSKQQEIYQELTRLKAAVGELPEKSKQQEIYQELTQLKAAVERLCHPCPWEW
TFFQGNCYFMSNSQRNWHDSITACKEVGAQLVVIKSAEEQNFLQLQSSRSNRFTWM
GLSDLNQEGTWQWVDGSPLLPSFKQYWNRGEPNNVGEEDCAEFSGNGWNDDKCN
LAKFWICKKSAASCSRDEEQFLSPAPATPNPPPA
SEQ ID NO: 41
QSRQDAIYQNLTQLKAAVGELSEKSKLQEIYQELTQLKAAVERLCHPCPWEWTFFQ
GNCYFMSNSQRNWHDSITACKEVGAQLVVIKSAEEQNFLQLQSSRSNRFTWMGLSD
LNQEGTWQWVDGSPLLPSFKQYWNRGEPNNVGEEDCAEFSGNGWNDDKCNLAKF
WICKKSAASCSRDEEQFLSPAPATPNPPPA
SEQ ID NO: 42
MEYHPDLENL DEDGYTQLHF DSQSNTRIAV VSEKGSCAAS PPWRLIAVIL
GILCLVILVIAVVLGTMAIW RSNSGSNTLE NGYFLSRNKE NHSQPTQSSL
EDSVTPTKAV KTTGVLSSPCPPNWIIYEKS CYLFSMSLNS WDGSKRQCWQ
LGSNLLKIDS SNELGFIVKQ VSSQPDNSFWIGLSRPQTEV PWLWEDGSTF
SSNLFQIRTT ATQENPSPNC VWIHVSVIYD QLCSVPSYSICEKKFSM
SEQ ID NO: 43
MMQEQQPQST EKRGWLSLRL WSVAGISIAL LSACFIVSCV VTYHFTY GET
GKRLSELHSYHSSLTCFSEG TKVPAWGCCP ASWKSFGSSC YFISSEEKVW
SKSEQNCVEM GAHLVVENTEAEQNFIVQQL NESFSYFLGL SDPQGNNNWQ
WIDKTPYEKN VRFWHLGEPN HSAEQCASIVFWKPTGWGWN DVICETRRNS
ICEMNKIYL
SEQ ID NO: 44
MGLEKPQSKLEGGMHPQLIPSVIAVVFILLLSVCFIASCLVTHHNFSRCKRGTGVHKL
EHHAKLKCIKEKSELKSAEGSTWNCCPIDWRAFQSNCYFPLTDNKTWAESERNCSG
MGAHLMTISTEAEQNFIIQFLDRRLSYFLGLRDENAKGQWRWVDQTPFNPRRVFWH
KNEPDNSQGENCVVLVYNQDKWAWNDVPCNFEASRICKIPGTTLN

1. A liposome comprising a targeting molecule that binds a target antigen expressed by a pathogen, wherein the targeting molecule is a Dendritic Cell-Specific Intercellular adhesion molecule-3-Grabbing Non-integrin (DC-SIGN) polypeptide or a fragment thereof comprising a carbohydrate recognition domain (CRD), and wherein the targeting molecule is incorporated into the outer surface of the liposome.

2. The liposome of claim 1, wherein the DC-SIGN polypeptide fragment comprises an amino acid sequence having at least 90% identity to a DC-SIGN polypeptide comprising CRD (SEQ ID NO: 1) and one or more neck regions of DC-SIGN selected from the group consisting of (NR1) SEQ ID NO: 2, (NR2) SEQ ID NO: 3, (NR3) SEQ ID NO: 4, (NR4) SEQ ID NO: 5, (NR5) SEQ ID NO: 6, (NR6) SEQ ID NO: 7, (NR7) SEQ ID NO: 8 and (NR8) SEQ ID NO: 9.

3. The liposome of claim 2, wherein the fragment comprises SEQ ID NO: 1, SEQ ID NO: 8 and SEQ ID NO: 9.

4. The liposome of claim 3, wherein the fragment comprises an amino acid sequence having at least 90% identity to SEQ ID NO: 10.

5. The liposome of claim 3, wherein the fragment comprises an amino acid sequence having at least 90% identity to SEQ ID NO: 40.

6. The liposome of claim 2, wherein the fragment comprises SEQ ID NO: 1, SEQ ID NO: 2 and SEQ ID NO: 3.

7. The liposome of claim 6, wherein the fragment comprises an amino acid sequence having at least 90% identity to SEQ ID NO: 12.

8. The liposome of claim 6, wherein the fragment comprises an amino acid sequence having at least 90% identity to SEQ ID NO: 41.

9. The liposome of claim 1, wherein the liposome does not comprise an antipathogenic agent.

10. The liposome of claim 1, further comprising an antipathogenic agent, wherein the antipathogenic agent is encapsulated in the liposome.

11. The method of claim 10, wherein the antipathogenic agent is an antiviral agent, an antifungal agent, an antibacterial agent, an antiprotozoan agent or an anthelminthic agent.

12.-30. (canceled)

31. A liposome comprising an antibacterial agent and a targeting molecule that binds a target antigen on a bacterial cell, wherein the targeting molecule is incorporated into the outer surface of the liposome and the antibacterial agent is encapsulated in the liposome, wherein the targeting molecule is selected from the group consisting of Dectin-1 or a fragment thereof, Dectin-2 or a fragment thereof, Dectin-3 or a fragment thereof, and DC-SIGN or a fragment thereof.

32. The liposome of claim 31, wherein the targeting molecule comprises an amino acid sequence having at least 90% identity to SEQ ID NO: 20, SEQ ID NO: 21, SEQ ID NO: 22, SEQ ID NO: 40 or SEQ ID NO: 41.

33. The liposome of claim 31, wherein the bacterial cell is a mycobacterial cell.

34. The liposome of claim 31, wherein the mycobacterial cell is a Mycobacterium tuberculosis cell, a Mycobacterium avium cell or a Mycobacterium ulcerans cell.

35. The liposome of claim 31, wherein the antibacterial agent is an antibiotic.

36. (canceled)

37. (canceled)

38. A plurality of liposomes according to claim 31.

39.-44. (canceled)

45. A pharmaceutical composition comprising the plurality of liposomes of claim 38.

46. A method of treating or preventing an infection in a subject comprising administering to the subject having an infection or at risk of developing an infection an effective amount of the plurality of liposomes of claim 38.

47. The method of claim 46, wherein the infection is a viral infection, a parasitic infection or a bacterial infection.

48. The method of claim 46, wherein the infection is not a fungal infection.

49. The method of claim 47, wherein the fungal infection is an Aspergillus infection, a Cryptococcus infection, a Candida infection or a Trichophyton infection.

50. The method of claim 47, wherein the viral infection is a coronavirus.

51. The method of claim 50, wherein the coronavirus is SARS-COV2 virus.

52. The method of claim 47, wherein the bacterial infection is a Mycobacterium tuberculosis infection, a Mycobacterium avium infection or a Mycobacterium ulcerans infection.

53. The method of claim 47, wherein the parasitic infection is a Toxoplasma gondii infection.

54. The method of claim 46, wherein the subject is immunocompromised.

55. The method of claim 46, wherein the subject has pneumonia, asthma, COPD, AIDS, cystic fibrosis, tuberculosis, emphysema, sarcoidosis or is taking an immunosuppressive drug.

56.-70. (canceled)

71. A composition comprising:
a) a DC-SIGN polypeptide or a fragment thereof, wherein the fragment comprises the CRD domain of DC-SIGN; and
b) a renaturation buffer.

72. The composition of claim 71, wherein the renaturation buffer comprises between about 0.5M L-arginine and 1.5M L-arginine.

73. The composition of claim 71, wherein the fragment comprises a polypeptide sequence comprising SEQ ID NO: 1.

74. The composition of claim 73, wherein the fragment comprises SEQ ID NO: 1 and one or more neck regions of DC-SIGN selected from the group consisting of (NR1) SEQ ID NO: 2, (NR2) SEQ ID NO: 3, (NR3) SEQ ID NO: 4, (NR4) SEQ ID NO: 5, (NR5) SEQ ID NO: 6, (NR6) SEQ ID NO: 7, (NR7) SEQ ID NO: 8 and (NR8) SEQ ID NO: 9.

75. The composition of claim 74, wherein the fragment comprises SEQ ID NO: 1, SEQ ID NO: 8 and SEQ ID NO: 9.

76. The composition of claim 75, wherein the fragment comprises SEQ ID NO: 10.

77. The composition of claim 74, wherein the fragment comprises SEQ ID NO: 1, SEQ ID NO: 2 and SEQ ID NO: 3.

78. The composition of claim 76, wherein the fragment comprises SEQ ID NO: 12.

79.-84. (canceled)


SEQUENCE LISTING (44 sequences)

SEQ ID NO: 1 - protein, 152 aa, synthetic construct
CHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 2 - protein, 21 aa, synthetic construct
QSRQDAIYQNLTQLKAAVGEL

SEQ ID NO: 3 - protein, 23 aa, synthetic construct
SEKSKLQEIYQELTQLKAAVGEL

SEQ ID NO: 4 - protein, 23 aa, synthetic construct
PEKSKLQEIYQELTRLKAAVGEL

SEQ ID NO: 5 - protein, 23 aa, synthetic construct
PEKSKLQEIYQELTWLKAAVGEL

SEQ ID NO: 6 - protein, 23 aa, synthetic construct
PEKSKMQEIYQELTRLKAAVGEL

SEQ ID NO: 7 - protein, 23 aa, synthetic construct
PEKSKQQEIYQELTRLKAAVGEL

SEQ ID NO: 8 - protein, 23 aa, synthetic construct
PEKSKQQEIYQELTRLKAAVGEL

SEQ ID NO: 9 - protein, 23 aa, synthetic construct
PEKSKQQEIYQELTQLKAAVERL

SEQ ID NO: 10 - protein, 223 aa, synthetic construct
MAHHHHHHYGTGSGKGKGSGSGGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 11 - DNA, 682 bp, synthetic construct
  source 1..682: mol_type = other DNA
ggtaccatggctcaccatcaccaccaccattatggaactggttctggcaagggcaagggcagcggcagcggtggagaactacccgagaagtcaaaacagcaagagatttaccaggagttgactcgtctgaaggcggcggtgggcgaacttccggaaaaatcgaaacagcaggagatctaccaagagttgacgcagttgaaggcggcggttgaacgtctgtgtcatccgtgtccgtgggaatggaccttcttccagggcaactgctatttcatgtctaacagccagagaaattggcacgacagcattaccgcatgtaaagaagttggtgcacagctggtggtgatcaaatctgcggaggaacaaaactttctccaactgcaatccagccgtagcaatcgttttacctggatgggtctgagcgacctgaatcaggagggcacctggcagtgggttgacggctcgccgctgctgccatcatttaaacaatattggaaccgcggtgaaccgaacaacgtcggtgaggaagattgcgccgagttcagcggtaacggctggaacgatgacaagtgcaatctggcaaagttctggatctgcaagaaatccgcggccagctgcagccgcgatgaagagcaatttttatccccggctccggcgaccccgaatccgcctccggcttaattaa

SEQ ID NO: 12 - protein, 218 aa, synthetic construct
MAHHHHHHYGTGSGKGKGSGSGQSRQDAIYQNLTQLKAAVGELSEKSKLQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 13 - DNA, 667 bp, synthetic construct
  source 1..667: mol_type = other DNA
ggtaccatggctcaccatcaccaccaccattatggaactggttctggcaagggtaagggtagcggctctggccagagcagacaagacgcaatttaccagaacctgacccagcttaaggcggcggttggcgagctgtccgaaaaaagcaaactgcaagagatctaccaagagttgacgcagttgaaggctgctgtggaacgtctgtgccatccgtgtccgtgggaatggacctttttccagggtaactgctatttcatgagcaacagccagcgtaattggcacgactcgatcaccgcgtgcaaagaagttggtgcacagctggtcgtgatcaaaagcgcggaggaacaaaactttttgcaactccaatcaagtcgctccaatcgtttcacctggatgggtctgtcggatctgaatcaggagggcacctggcaatgggttgatggcagcccgctgctgccgagctttaaacagtattggaaccgcggtgagccgaataacgtgggcgaagaagattgtgcggagttcagcggtaatggttggaacgacgacaagtgcaacctggcgaaattctggatttgtaaaaagtctgcagcctcctgcagccgtgatgaagagcagtttttgtccccggcgccagctacgccgaacccgccgcctgcttaattaa

SEQ ID NO: 14 - protein, 356 aa, synthetic construct
MAHHHHHHYGTGSGKGKGSGSGQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 15 - DNA, 1068 bp, synthetic construct
  source 1..1068: mol_type = other DNA
atggctcaccatcaccaccaccattatggaaccggtagcggcaagggtaaaggctctggttctggtcagagccgccaggatgcgatttatcaaaacctgacccaactgaaggctgctgtgggtgaactttcagaaaagagcaagttacaagaaatttaccaagagctcacgcagctgaaagcggccgttggcgagttgccggagaagtcgaagctgcaagagatttaccaagaacttacccgtttgaaggctgcggtaggggagctgccggaaaaatccaaactgcaagaaatctaccaggagctcacgtggctgaaggctgcggtgggcgagttgccagaaaagtccaaaatgcaggagatctaccaagagctgacgcgtttaaaggcggcggtcggcgagctgccggaaaaatctaagcaacaggagatctaccaggagttgactcgcttaaaggcggctgtgggcgagttgccggaaaagtccaagcaacaagagatctaccaagagttaacacgtttgaaagcggcggtcggtgagttgccagaaaagtcgaaacagcaagagatctatcaggaactgacccagctcaaagccgccgtggaaagactgtgccatccgtgtccgtgggaatggacctttttccaaggtaactgctatttcatgagcaacagccagcgtaattggcacgacagcattaccgcatgtaaagaagttggcgcacagctggttgtgattaaatctgcagaagaacaaaattttctgcagctgcagagcagccgctcgaaccgtttcacctggatgggtctgtccgacctgaatcaggagggcacctggcagtgggttgatggttcgccgctgctgccgagctttaaacagtattggaatcgcggtgagccgaacaacgttggcgaggaagattgtgcggaatttagcggcaacggttggaacgacgacaaatgcaatctggcaaaattctggatctgcaagaaatccgcagcgagttgcagccgtgatgaagagcagttcctgagcccggcgccggcgaccccgaacccgcctccggct

SEQ ID NO: 16 - protein, 404 aa, synthetic construct
MSDSKEPRLQQLGLLEEEQLRGLGFRQTRGYKSLAGCLGHGPLVLQLLSFTLLAGLLVQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 17 - protein, 70 aa, synthetic construct
MSDSKEPRLQQLGLLEEEQLRGLGFRQTRGYKSLAGCLGHGPLVLQLLSFTLLAGLLVQVSKVPSSISQE

SEQ ID NO: 18 - protein, 5 aa, synthetic construct
GSGSG

SEQ ID NO: 19 - protein, 6 aa, synthetic construct
HHHHHH

SEQ ID NO: 20 - protein, 179 aa, synthetic construct
IWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 21 - protein, 168 aa, synthetic construct
TYHFTYGETGKRLSELHSYHSSLTCFSEGTKVPAWGCCPASWKSFGSSCYFISSEEKVWSKSEQNCVEMGAHLVVFNTEAEQNFIVQQLNESFSYFLGLSDPQGNNNWQWIDKTPYEKNVRFWHLGEPNHSAEQCASIVFWKPTGWGWNDVICETRRNSICEMNKIYL

SEQ ID NO: 22 - protein, 172 aa, synthetic construct
HNFSRCKRGTGVHKLEHHAKLKCIKEKSELKSAEGSTWNCCPIDWRAFQSNCYFPLTDNKTWAESERNCSGMGAHLMTISTEAEQNFIIQFLDRRLSYFLGLRDENAKGQWRWVDQTPFNPRRVFWHKNEPDNSQGENCVVLVYNQDKWAWNDVPCNFEASRICKIPGTTLN

SEQ ID NO: 23 - protein, 176 aa, synthetic construct
FWRHNSGRNPEEKDSFLSRNKENHKPTESSLDEKVAPSKASQTTGGFSQSCLPNWIMHGKSCYLFSFSGNSWYGSKRHCSQLGAHLLKIDNSKEFEFIESQTSSHRINAFWIGLSRNQSEGPWFWEDGSAFFPNSFQVRNAVPQESLLHNCVWIHGSEVYNQICNTSSYSICEKEL

SEQ ID NO: 24 - protein, 166 aa, synthetic construct
IMDQPSRRLYELHTYHSSLTCFSEGTMVSEKMWGCCPNHWKSFGSSCYLISTKENFWSTSEQNCVQMGAHLVVINTEAEQNFITQQLNESLSYFLGLSDPQGNGKWQWIDDTPFSQNVRFWHPHEPNLPEERCVSIVYWNPSKWGWNDVFCDSKHNSICEMKKIYL

SEQ ID NO: 25 - protein, 176 aa, synthetic construct
HYFLRWTRGSVVKLSDYHTRVTCIREEPQPGATGGTWTCCPVSWRAFQSNCYFPLNDNQTWHESERNCSGMSSHLVTINTEAEQNFVTQLLDKRFSYFLGLADENVEGQWQWVDKTPFNPHTVFWEKGESNDFMEEDCVVLVHVHEKWVWNDFPCHFEVRRICKLPGITFNWKPSK

SEQ ID NO: 26 - DNA, 604 bp, synthetic construct
  source 1..604: mol_type = other DNA
atggcacatcaccaccaccatcacgtgggtaccggcagcggcaagggcaagggcagcggcagcggtttttggcgtcacaacagcggtcgtaacccggaggagaaagacaacttcctgagccgtaacaaggagaaccacaaaccgaccgagagcagcctggacgaaaaggttgcgccgagcaaagcgagccagaccaccggtggcttcagccaaccgtgcctgccgaactggatcatgcacggcaagagctgctacctgttcagctttagcggtaacagctggtatggcagcaaacgtcattgcagccagctgggtgcgcacctgctgaagatcgacaacagcaaagagttcgaatttattgagagccagaccagcagccaccgtatcaacgcgttttggattggtctgagccgtaaccaaagcgagggtccgtggttctgggaagatggcagcgcgttctttccgaacagctttcaagtgcgtaacaccgcgccgcaagaaagcctgctgcacaactgcgtttggattcacggcagcgaggtttacaatcaaatctgcaataccagcagctacagcatctgcgagaaggaactggcttaattaa

SEQ ID NO: 27 - protein, 199 aa, synthetic construct
MAHHHHHHVGTGSGKGKGSGSGFWRHNSGRNPEEKDSFLSRNKENHKPTESSLDEKVAPSKASQTTGGFSQSCLPNWIMHGKSCYLFSFSGNSWYGSKRHCSQLGAHLLKIDNSKEFEFIESQTSSHRINAFWIGLSRNQSEGPWFWEDGSAFFPNSFQVRNAVPQESLLHNCVWIHGSEVYNQICNTSSYSICEKELA

SEQ ID NO: 28 - DNA, 574 bp, synthetic construct
  source 1..574: mol_type = other DNA
atggcacatcaccaccaccatcacgtgggtaccggcagcggcaagggcaagggcagcggcagcggtataatggatcaaccctcaagaaggctatatgagctgcacacgtaccacagctccctcacgtgcttttctgagggtactatggtgtccgagaaaatgtggggctgctgcccgaatcattggaaatcttttggtagcagctgttatctgatcagcaccaaagagaacttctggagtaccagcgagcaaaactgcgtccagatgggcgcacacctggttgtgattaacaccgaagcggaacagaacttcatcacccagcaattaaatgaaagcttgtcttacttcctgggtctgtcggatccgcagggcaacggcaagtggcagtggattgacgacaccccgttctcccaaaacgtgcgcttttggcatccgcatgaaccgaatctgccggaagaacgttgtgtaagcattgtttattggaatccaagcaagtggggttggaacgacgttttttgtgatagcaagcacaactcgatctgcgagatgaaaaagatctacttggcttaattaa

SEQ ID NO: 29 - protein, 189 aa, synthetic construct
MAHHHHHHVGTGSGKGKGSGSGIMDQPSRRLYELHTYHSSLTCFSEGTMVSEKMWGCCPNHWKSFGSSCYLISTKENFWSTSEQNCVQMGAHLVVINTEAEQNFITQQLNESLSYFLGLSDPQGNGKWQWIDDTPFSQNVRFWHPHEPNLPEERCVSIVYWNPSKWGWNDVFCDSKHNSICEMKKIYLA

SEQ ID NO: 30 - DNA, 604 bp, synthetic construct
  source 1..604: mol_type = other DNA
atggctcaccatcaccaccaccattatggtaccggttccggcaaaggcaagggctccggctctggtcactatttcctgcgttggacccgcggttccgtggtgaaactgagcgactaccatacgcgcgtgacttgcattcgtgaagagccgcagccgggcgcaaccggcggtacatggacgtgctgcccggttagctggcgtgcgttccagtctaactgttatttcccactgaatgacaaccaaacgtggcatgagagcgaacgtaactgcagcggcatgagcagtcacctggttaccattaacaccgaggcggagcaaaactttgtgacccaattgctcgacaagcgcttcagctacttcctgggtttggccgatgaaaatgttgagggtcagtggcagtgggtagataagaccccgtttaatccgcacaccgtcttttgggaaaagggtgagtcgaacgacttcatggaagaagattgtgttgttctggtgcacgtgcacgagaagtgggtttggaatgatttcccgtgtcattttgaagtcagacgtatctgcaaattaccgggtatcacctttaactggaaaccgagcaaagcttaattaa

SEQ ID NO: 31 - protein, 199 aa, synthetic construct
MAHHHHHHVGTGSGKGKGSGSGHYFLRWTRGSVVKLSDYHTRVTCIREEPQPGATGGTWTCCPVSWRAFQSNCYFPLNDNQTWHESERNCSGMSSHLVTINTEAEQNFVTQLLDKRFSYFLGLADENVEGQWQWVDKTPFNPHTVFWEKGESNDFMEEDCVVLVHVHEKWVWNDFPCHFEVRRICKLPGITFNWKPSKA

SEQ ID NO: 32 - DNA, 649 bp, synthetic construct
  source 1..649: mol_type = other DNA
atggcacatcaccaccaccatcacgtgggtaccggttcgaatgatgacgacgacaagagtccggatcccggaagtggaaaaggcaagggttcagggtctggcatctggcgtagcaacagcggtagcaataccctggaaaacggttactttttgagccgcaacaaggagaaccattctcagccgacgcaaagcagcttggaggactcggtgaccccaacgaaagctgtgaagaccacgggtgtcctgtcatccccgtgcccgccgaattggatcatctatgagaaaagctgctatctctttagcatgagcctgaatagctgggacggctccaaacgtcagtgttggcagctgggctcgaacttgctgaagatcgactcctctaacgaattaggtttcattgttaagcaagttagcagccaaccggataattccttttggattggcctgtctcgtccgcagaccgaagttccgtggctgtgggaagatggttccactttcagctctaacctgttccagattcgcaccaccgcaacccaagagaatcctagtccgaactgcgtttggattcacgtgagcgtgatctacgatcagctgtgttccgtcccgagctactcgatctgcgagaaaaagttctctatggcttaattaa

SEQ ID NO: 33 - protein, 214 aa, synthetic construct
MAHHHHHHYGTGSNDDDDKSPDPGSGKGKGSGSGIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSMA

SEQ ID NO: 34 - DNA, 616 bp, synthetic construct
  source 1..616: mol_type = other DNA
atggcacatcaccaccaccatcacgtgggtaccggttcgaatgatgacgacgacaagagtccggatcccgggtctggaaaaggcaagggaagtggttcaggcacctaccactttacctacggcgaaaccggtaaacgtctgtccgagctccactcatatcactcctctctgacgtgctttagcgagggtactaaagtgccagcgtggggttgttgtccggcgagctggaagtcgttcggcagcagctgctatttcatcagctcggaggaaaaagtttggagcaagagcgagcaaaactgcgtggaaatgggtgcacatttggttgtcttcaacaccgaagcggagcaaaactttatcgtgcagcagctgaacgaaagcttctcctacttcctgggtctgtccgacccgcagggtaataacaactggcagtggattgataaaaccccgtatgaaaagaacgtgcgcttttggcatttgggcgagccgaatcattctgccgaacaatgtgcgagcattgttttctggaagccgaccggctggggttggaatgacgttatttgcgagacgcgtcgtaacagcatctgcgagatgaataaaatctacctggcttaattaa

SEQ ID NO: 35 - protein, 203 aa, synthetic construct
MAHHHHHHVGTGSNDDDDKSPDPGSGKGKGSGSGTYHFTYGETGKRLSELHSYHSSLTCFSEGTKVPAWGCCPASWKSFGSSCYFISSEEKVWSKSEQNCVEMGAHLVVFNTEAEQNFIVQQLNESFSYFLGLSDPQGNNNWQWIDKTPYEKNVRFWHLGEPNHSAEQCASIVFWKPTGWGWNDVICETRRNSICEMNKIYLA

SEQ ID NO: 36 - DNA, 628 bp, synthetic construct
  source 1..628: mol_type = other DNA
atggctcaccatcaccaccaccattatggtaccggttcgaatgatgacgacgacaagagtccggatcccgggtctggaaaaggcaagggaagtggttcaggccacaacttcagccgttgtaagcgcggtacgggcgtgcataagttggagcaccacgccaagctcaagtgcatcaaagaaaaatccgagctgaaatctgctgagggcagcacctggaactgctgcccgattgattggcgtgcgtttcaaagcaattgctacttcccgctgaccgataacaaaacctgggcggaaagcgagcgcaactgcagcggtatgggtgcacatctgatgaccatttcgaccgaagcggagcagaatttcatcatccaatttttggaccgccgtctgtcctacttcctgggtctgcgtgatgaaaatgcaaaaggccaatggcgttgggttgaccagaccccgtttaacccgcgtcgtgttttttggcataagaacgaaccagacaacagccagggtgaaaactgcgtcgtgttagtttataaccaggataaatgggcgtggaacgacgtgccgtgtaatttcgaggcttctcgcatttgtaagatcccgggtacgactctgaatgcttaattaa

SEQ ID NO: 37 - protein, 207 aa, synthetic construct
MAHHHHHHVGTGSNDDDDKSPDPGSGKGKGSGSGHNFSRCKRGTGVHKLEHHAKLKCIKEKSELKSAEGSTWNCCPIDWRAFQSNCYFPLTDNKTWAESERNCSGMGAHLMTISTEAEQNFIIQFLDRRLSYFLGLRDENAKGQWRWVDQTPFNPRRVFWHKNEPDNSQGENCVVLVYNQDKWAWNDVPCNFEASRICKIPGTTLNA

SEQ ID NO: 38-39 - skipped (2 entries)

SEQ ID NO: 40 - protein, 200 aa, synthetic construct
GELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPP

SEQ ID NO: 41 - protein, 195 aa, synthetic construct
QSRQDAIYQNLTQLKAAVGELSEKSKLQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPP

SEQ ID NO: 42 - protein, 247 aa, synthetic construct
MEYHPDLENLDEDGYTQLHFDSQSNTRIAVVSEKGSCAASPPWRLIAVILGILCLVILVIAVVLGTMAIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 43 - protein, 209 aa, synthetic construct
MMQEQQPQSTEKRGWLSLRLWSVAGISIALLSACFIVSCVVTYHFTYGETGKRLSELHSYHSSLTCFSEGTKVPAWGCCPASWKSFGSSCYFISSEEKVWSKSEQNCVEMGAHLVVFNTEAEQNFIVQQLNESFSYFLGLSDPQGNNNWQWIDKTPYEKNVRFWHLGEPNHSAEQCASIVFWKPTGWGWNDVICETRRNSICEMNKIYL

SEQ ID NO: 44 - protein, 215 aa, synthetic construct
MGLEKPQSKLEGGMHPQLIPSVIAVVFILLLSVCFIASCLVTHHNFSRCKRGTGVHKLEHHAKLKCIKEKSELKSAEGSTWNCCPIDWRAFQSNCYFPLTDNKTWAESERNCSGMGAHLMTISTEAEQNFIIQFLDRRLSYFLGLRDENAKGQWRWVDQTPFNPRRVFWHKNEPDNSQGENCVVLVYNQDKWAWNDVPCNFEASRICKIPGTTLN